SEQUENCE LISTING (57 sequences)

SEQ ID NO: 1 - protein, 514 aa, synthetic construct
  REGION 1..514: note = CAR-C1
EVQFVESGGGLVQPGGSLKLSCAASGVTFSEAWMDWVRQASGKGLEWVAEIRDKPNSYVTYYAESVKGRFTISRDDSKSTAYLQMNSLKTEDTAIYYCTGAMDYWGQGTLVTVSSGGGGSGGGGSGGGGSDIVMTQTPLSLSVTIGQPASISCKSSQSLLYSNGKTYLNWLLQKPGQSPKRLIYQVSKLDPGIPDRFSGSGSETDFTLKISRVEAEDVGIYYCMQGSSLPWTFGGGTKVEIKRSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 2 - protein, 517 aa, synthetic construct
  REGION 1..517: note = CAR-C2
EVKFEESGGGLVQPGGSIKLSCAASGVTFSEAWMDWVRQSPEKGLEWVAEIRDKPNSYVTYYAESVKGRFTISRDDSKSRVYLQMNNLRTEDTGIYYCTGAMDYWGQGTSVTVSSGSTGGGGSGGGGSGGGGSDIVMTQTPLSLSVTIGQPASISCKSSQSLLYSNGKTYLNWLQQRPGQSPKRLIYQVSKLDPGIPDRFSGSGSETDFTLKISRVEAEDLGIYYCMQGSSLPWTFGGGTKLEIKRSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 3 - protein, 524 aa, synthetic construct
  REGION 1..524: note = CAR-C3
EVQFVESGGGLVQPGGSLKLSCAASGVTFSEAWMDWVRQASGKGLEWVAEIRDKPNSYVTYYAESVKGRFTISRDDSKSTAYLQMNSLKTEDTAIYYCTGAMDYWGQGTLVTVSSGGGGSGGGGSGGGGSDIVMTQTPLSLSVTIGQPASISCKSSQSLLYSNGKTYLNWLLQKPGQSPKRLIYQVSKLDPGIPDRFSGSGSETDFTLKISRVEAEDVGIYYCMQGSSLPWTFGGGTKVEIKASSHFVPVFLPAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 4 - protein, 527 aa, synthetic construct
  REGION 1..527: note = CAR-C4
EVKFEESGGGLVQPGGSIKLSCAASGVTFSEAWMDWVRQSPEKGLEWVAEIRDKPNSYVTYYAESVKGRFTISRDDSKSRVYLQMNNLRTEDTGIYYCTGAMDYWGQGTSVTVSSGSTGGGGSGGGGSGGGGSDIVMTQTPLSLSVTIGQPASISCKSSQSLLYSNGKTYLNWLQQRPGQSPKRLIYQVSKLDPGIPDRFSGSGSETDFTLKISRVEAEDLGIYYCMQGSSLPWTFGGGTKLEIKASSHFVPVFLPAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 5 - protein, 472 aa, synthetic construct
  REGION 1..472: note = CAR-C5
EVQFVESGGGLVQPGGSLKLSCAASGVTFSEAWMDWVRQASGKGLEWVAEIRDKPNSYVTYYAESVKGRFTISRDDSKSTAYLQMNSLKTEDTAIYYCTGAMDYWGQGTLVTVSSGGGGSGGGGSGGGGSDIVMTQTPLSLSVTIGQPASISCKSSQSLLYSNGKTYLNWLLQKPGQSPKRLIYQVSKLDPGIPDRFSGSGSETDFTLKISRVEAEDVGIYYCMQGSSLPWTFGGGTKVEIKRSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 6 - protein, 475 aa, synthetic construct
  REGION 1..475: note = CAR-C6
EVKFEESGGGLVQPGGSIKLSCAASGVTFSEAWMDWVRQSPEKGLEWVAEIRDKPNSYVTYYAESVKGRFTISRDDSKSRVYLQMNNLRTEDTGIYYCTGAMDYWGQGTSVTVSSGSTGGGGSGGGGSGGGGSDIVMTQTPLSLSVTIGQPASISCKSSQSLLYSNGKTYLNWLQQRPGQSPKRLIYQVSKLDPGIPDRFSGSGSETDFTLKISRVEAEDLGIYYCMQGSSLPWTFGGGTKLEIKRSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 7 - protein, 482 aa, synthetic construct
  REGION 1..482: note = CAR-C7
EVQFVESGGGLVQPGGSLKLSCAASGVTFSEAWMDWVRQASGKGLEWVAEIRDKPNSYVTYYAESVKGRFTISRDDSKSTAYLQMNSLKTEDTAIYYCTGAMDYWGQGTLVTVSSGGGGSGGGGSGGGGSDIVMTQTPLSLSVTIGQPASISCKSSQSLLYSNGKTYLNWLLQKPGQSPKRLIYQVSKLDPGIPDRFSGSGSETDFTLKISRVEAEDVGIYYCMQGSSLPWTFGGGTKVEIKASSHFVPVFLPAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 8 - protein, 485 aa, synthetic construct
  REGION 1..485: note = CAR-C8
EVKFEESGGGLVQPGGSIKLSCAASGVTFSEAWMDWVRQSPEKGLEWVAEIRDKPNSYVTYYAESVKGRFTISRDDSKSRVYLQMNNLRTEDTGIYYCTGAMDYWGQGTSVTVSSGSTGGGGSGGGGSGGGGSDIVMTQTPLSLSVTIGQPASISCKSSQSLLYSNGKTYLNWLQQRPGQSPKRLIYQVSKLDPGIPDRFSGSGSETDFTLKISRVEAEDLGIYYCMQGSSLPWTFGGGTKLEIKASSHFVPVFLPAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 9 - protein, 473 aa, synthetic construct
  REGION 1..473: note = CAR-C9
EVQFVESGGGLVQPGGSLKLSCAASGVTFSEAWMDWVRQASGKGLEWVAEIRDKPNSYVTYYAESVKGRFTISRDDSKSTAYLQMNSLKTEDTAIYYCTGAMDYWGQGTLVTVSSGGGGSGGGGSGGGGSDIVMTQTPLSLSVTIGQPASISCKSSQSLLYSNGKTYLNWLLQKPGQSPKRLIYQVSKLDPGIPDRFSGSGSETDFTLKISRVEAEDVGIYYCMQGSSLPWTFGGGTKVEIKRSVTVSSAAAIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 10 - protein, 476 aa, synthetic construct
  REGION 1..476: note = CAR-C10
EVKFEESGGGLVQPGGSIKLSCAASGVTFSEAWMDWVRQSPEKGLEWVAEIRDKPNSYVTYYAESVKGRFTISRDDSKSRVYLQMNNLRTEDTGIYYCTGAMDYWGQGTSVTVSSGSTGGGGSGGGGSGGGGSDIVMTQTPLSLSVTIGQPASISCKSSQSLLYSNGKTYLNWLQQRPGQSPKRLIYQVSKLDPGIPDRFSGSGSETDFTLKISRVEAEDLGIYYCMQGSSLPWTFGGGTKLEIKRSVTVSSAAAIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 11 - protein, 483 aa, synthetic construct
  REGION 1..483: note = CAR-C11
EVQFVESGGGLVQPGGSLKLSCAASGVTFSEAWMDWVRQASGKGLEWVAEIRDKPNSYVTYYAESVKGRFTISRDDSKSTAYLQMNSLKTEDTAIYYCTGAMDYWGQGTLVTVSSGGGGSGGGGSGGGGSDIVMTQTPLSLSVTIGQPASISCKSSQSLLYSNGKTYLNWLLQKPGQSPKRLIYQVSKLDPGIPDRFSGSGSETDFTLKISRVEAEDVGIYYCMQGSSLPWTFGGGTKVEIKASSHFVPVFLPAKPTTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 12 - protein, 486 aa, synthetic construct
  REGION 1..486: note = CAR-C12
EVKFEESGGGLVQPGGSIKLSCAASGVTFSEAWMDWVRQSPEKGLEWVAEIRDKPNSYVTYYAESVKGRFTISRDDSKSRVYLQMNNLRTEDTGIYYCTGAMDYWGQGTSVTVSSGSTGGGGSGGGGSGGGGSDIVMTQTPLSLSVTIGQPASISCKSSQSLLYSNGKTYLNWLQQRPGQSPKRLIYQVSKLDPGIPDRFSGSGSETDFTLKISRVEAEDLGIYYCMQGSSLPWTFGGGTKLEIKASSHFVPVFLPAKPTTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 13 - protein, 468 aa, synthetic construct
  REGION 1..468: note = CAR-C13
EVQFVESGGGLVQPGGSLKLSCAASGVTFSEAWMDWVRQASGKGLEWVAEIRDKPNSYVTYYAESVKGRFTISRDDSKSTAYLQMNSLKTEDTAIYYCTGAMDYWGQGTLVTVSSGGGGSGGGGSGGGGSDIVMTQTPLSLSVTIGQPASISCKSSQSLLYSNGKTYLNWLLQKPGQSPKRLIYQVSKLDPGIPDRFSGSGSETDFTLKISRVEAEDVGIYYCMQGSSLPWTFGGGTKVEIKRSVTVSSAAAIEVMYPPPYLDNEKSNGTIIHVKGKHLCPSPLFPGPSKPFWVLVVVGGVLACYSLLVTVAFIIFWVRRDQRLPPDAHKPPGGGSFRTPIQEEQADAHSTLAKIRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 14 - protein, 471 aa, synthetic construct
  REGION 1..471: note = CAR-C14
EVKFEESGGGLVQPGGSIKLSCAASGVTFSEAWMDWVRQSPEKGLEWVAEIRDKPNSYVTYYAESVKGRFTISRDDSKSRVYLQMNNLRTEDTGIYYCTGAMDYWGQGTSVTVSSGSTGGGGSGGGGSGGGGSDIVMTQTPLSLSVTIGQPASISCKSSQSLLYSNGKTYLNWLQQRPGQSPKRLIYQVSKLDPGIPDRFSGSGSETDFTLKISRVEAEDLGIYYCMQGSSLPWTFGGGTKLEIKRSVTVSSAAAIEVMYPPPYLDNEKSNGTIIHVKGKHLCPSPLFPGPSKPFWVLVVVGGVLACYSLLVTVAFIIFWVRRDQRLPPDAHKPPGGGSFRTPIQEEQADAHSTLAKIRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 15 - protein, 478 aa, synthetic construct
  REGION 1..478: note = CAR-C15
EVQFVESGGGLVQPGGSLKLSCAASGVTFSEAWMDWVRQASGKGLEWVAEIRDKPNSYVTYYAESVKGRFTISRDDSKSTAYLQMNSLKTEDTAIYYCTGAMDYWGQGTLVTVSSGGGGSGGGGSGGGGSDIVMTQTPLSLSVTIGQPASISCKSSQSLLYSNGKTYLNWLLQKPGQSPKRLIYQVSKLDPGIPDRFSGSGSETDFTLKISRVEAEDVGIYYCMQGSSLPWTFGGGTKVEIKASSHFVPVFLPAKPTTTPAPRPPTPAPTIASQPLSLRPEACRPAAGGAVHTRGLDFACDFWVLVVVGGVLACYSLLVTVAFIIFWVRRDQRLPPDAHKPPGGGSFRTPIQEEQADAHSTLAKIRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 16 - protein, 481 aa, synthetic construct
  REGION 1..481: note = CAR-C16
EVKFEESGGGLVQPGGSIKLSCAASGVTFSEAWMDWVRQSPEKGLEWVAEIRDKPNSYVTYYAESVKGRFTISRDDSKSRVYLQMNNLRTEDTGIYYCTGAMDYWGQGTSVTVSSGSTGGGGSGGGGSGGGGSDIVMTQTPLSLSVTIGQPASISCKSSQSLLYSNGKTYLNWLQQRPGQSPKRLIYQVSKLDPGIPDRFSGSGSETDFTLKISRVEAEDLGIYYCMQGSSLPWTFGGGTKLEIKASSHFVPVFLPAKPTTTPAPRPPTPAPTIASQPLSLRPEACRPAAGGAVHTRGLDFACDFWVLVVVGGVLACYSLLVTVAFIIFWVRRDQRLPPDAHKPPGGGSFRTPIQEEQADAHSTLAKIRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 17 - protein, 479 aa, synthetic construct
  REGION 1..479: note = CAR-C23
EVQFVESGGGLVQPGGSLKLSCAASGVTFSEAWMDWVRQASGKGLEWVAEIRDKPNSYVTYYAESVKGRFTISRDDSKSTAYLQMNSLKTEDTAIYYCTGAMDYWGQGTLVTVSSGGGGSGGGGSGGGGSDIVMTQTPLSLSVTIGQPASISCKSSQSLLYSNGKTYLNWLLQKPGQSPKRLIYQVSKLDPGIPDRFSGSGSETDFTLKISRVEAEDVGIYYCMQGSSLPWTFGGGTKVEIKASSHFVPVFLPAKPTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 18 - protein, 462 aa, synthetic construct
  REGION 1..462: note = CAR-C27
EVKFEESGGGLVQPGGSIKLSCAASGVTFSEAWMDWVRQSPEKGLEWVAEIRDKPNSYVTYYAESVKGRFTISRDDSKSRVYLQMNNLRTEDTGIYYCTGAMDYWGQGTSVTVSSGGGGSDIVMTQTPLSLSVTIGQPASISCKSSQSLLYSNGKTYLNWLQQRPGQSPKRLIYQVSKLDPGIPDRFSGSGSETDFTLKISRVEAEDLGIYYCMQGSSLPWTFGGGTKLEIKRSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 19 - protein, 472 aa, synthetic construct
  REGION 1..472: note = CAR-C28
EVKFEESGGGLVQPGGSIKLSCAASGVTFSEAWMDWVRQSPEKGLEWVAEIRDKPNSYVTYYAESVKGRFTISRDDSKSRVYLQMNNLRTEDTGIYYCTGAMDYWGQGTSVTVSSGGGGSDIVMTQTPLSLSVTIGQPASISCKSSQSLLYSNGKTYLNWLQQRPGQSPKRLIYQVSKLDPGIPDRFSGSGSETDFTLKISRVEAEDLGIYYCMQGSSLPWTFGGGTKLEIKASSHFVPVFLPAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 20 - protein, 487 aa, synthetic construct
  REGION 1..487: note = CAR-C29
EVQFVESGGGLVQPGGSLKLSCAASGVTFSEAWMDWVRQASGKGLEWVAEIRDKPNSYVTYYAESVKGRFTISRDDSKSTAYLQMNSLKTEDTAIYYCTGAMDYWGQGTLVTVSSGGGGSGGGGSGGGGSGGGGSDIVMTQTPLSLSVTIGQPASISCKSSQSLLYSNGKTYLNWLLQKPGQSPKRLIYQVSKLDPGIPDRFSGSGSETDFTLKISRVEAEDVGIYYCMQGSSLPWTFGGGTKVEIKASSHFVPVFLPAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 21 - protein, 477 aa, synthetic construct
  REGION 1..477: note = CAR-C30
EVKFEESGGGLVQPGGSIKLSCAASGVTFSEAWMDWVRQSPEKGLEWVAEIRDKPNSYVTYYAESVKGRFTISRDDSKSRVYLQMNNLRTEDTGIYYCTGAMDYWGQGTSVTVSSGGGGSGGGGSDIVMTQTPLSLSVTIGQPASISCKSSQSLLYSNGKTYLNWLQQRPGQSPKRLIYQVSKLDPGIPDRFSGSGSETDFTLKISRVEAEDLGIYYCMQGSSLPWTFGGGTKLEIKASSHFVPVFLPAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 22 - protein, 21 aa, synthetic construct
  REGION 1..21: note = CD8ss
MALPVTALLLPLALLLHAARP

SEQ ID NO: 23 - protein, 115 aa, synthetic construct
  REGION 1..115: note = H3L3
EVQFVESGGGLVQPGGSLKLSCAASGVTFSEAWMDWVRQASGKGLEWVAEIRDKPNSYVTYYAESVKGRFTISRDDSKSTAYLQMNSLKTEDTAIYYCTGAMDYWGQGTLVTVSS

SEQ ID NO: 24 - protein, 115 aa, synthetic construct
  REGION 1..115: note = PP6373
EVKFEESGGGLVQPGGSIKLSCAASGVTFSEAWMDWVRQSPEKGLEWVAEIRDKPNSYVTYYAESVKGRFTISRDDSKSRVYLQMNNLRTEDTGIYYCTGAMDYWGQGTSVTVSS

SEQ ID NO: 25 - protein, 112 aa, synthetic construct
  REGION 1..112: note = H3L3
DIVMTQTPLSLSVTIGQPASISCKSSQSLLYSNGKTYLNWLLQKPGQSPKRLIYQVSKLDPGIPDRFSGSGSETDFTLKISRVEAEDVGIYYCMQGSSLPWTFGGGTKVEIK

SEQ ID NO: 26 - protein, 112 aa, synthetic construct
  REGION 1..112: note = PP6373
DIVMTQTPLSLSVTIGQPASISCKSSQSLLYSNGKTYLNWLQQRPGQSPKRLIYQVSKLDPGIPDRFSGSGSETDFTLKISRVEAEDLGIYYCMQGSSLPWTFGGGTKLEIK

SEQ ID NO: 27 - protein, 5 aa, synthetic construct
  REGION 1..5: note = GGGGS
GGGGS

SEQ ID NO: 28-29 - skipped (2 entries)

SEQ ID NO: 30 - protein, 4 aa, synthetic construct
  REGION 1..4: note = GGGS
GGGS

SEQ ID NO: 31 - protein, 5 aa, synthetic construct
  REGION 1..5: note = GSGGS
GSGGS

SEQ ID NO: 32 - protein, 39 aa, synthetic construct
  REGION 1..39: note = CD28
IEVMYPPPYLDNEKSNGTIIHVKGKHLCPSPLFPGPSKP

SEQ ID NO: 33 - protein, 57 aa, synthetic construct
  REGION 1..57: note = CD8
SHFVPVFLPAKPTTTPAPRPPTPAPTIASQPLSLRPEACRPAAGGAVHTRGLDFACD

SEQ ID NO: 34 - protein, 27 aa, synthetic construct
  REGION 1..27: note = CD28
FWVLVVVGGVLACYSLLVTVAFIIFWV

SEQ ID NO: 35 - protein, 24 aa, synthetic construct
  REGION 1..24: note = CD8
IYIWAPLAGTCGVLLLSLVITLYC

SEQ ID NO: 36 - protein, 42 aa, synthetic construct
  REGION 1..42: note = 41BB
KRGRKKLLYIFKQPFMRPVQTTQEEDGCSCRFPEEEEGGCEL

SEQ ID NO: 37 - protein, 41 aa, synthetic construct
  REGION 1..41: note = CD28
RSKRSRLLHSDYMNMTPRRPGPTRKHYQPYAPPRDFAAYRS

SEQ ID NO: 38 - protein, 37 aa, synthetic construct
  REGION 1..37: note = OX40
RRDQRLPPDAHKPPGGGSFRTPIQEEQADAHSTLAKI

SEQ ID NO: 39 - protein, 113 aa, synthetic construct
  REGION 1..113: note = CD3
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 40 - protein, 225 aa, synthetic construct
  REGION 1..225: note = RFP
MASSEDVIKEFMRFKVRMEGSVNGHEFEIEGEGEGRPYEGTQTAKLKVTKGGPLPFAWDILSPQFQYGSKAYVKHPADIPDYLKLSFPEGFKWERVMNFEDGGVVTVTQDSSLQDGEFIYKVKLRGTNFPSDGPVMQKKTMGWEASTERMYPEDGALKGEIKMRLKLKDGGHYDAEVKTTYMAKKPVQLPGAYKTDIKLDITSHNEDYTIVEQYERAEGRHSTGA

SEQ ID NO: 41 - DNA, 590 bp, synthetic construct
  misc_feature 1..590: note = IRES
  source 1..590: mol_type = other DNA
ccacctacatggggatccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatgacc

SEQ ID NO: 42 - protein, 10 aa, synthetic construct
  REGION 1..10: note = VH CDR1
GVTFSEAWMD

SEQ ID NO: 43 - protein, 19 aa, synthetic construct
  REGION 1..19: note = VH CDR2
EIRDKPNSYVTYYAESVKG

SEQ ID NO: 44 - protein, 4 aa, synthetic construct
  REGION 1..4: note = VH CDR3
AMDY

SEQ ID NO: 45 - protein, 16 aa, synthetic construct
  REGION 1..16: note = VL CDR1
KSSQSLLYSNGKTYLN

SEQ ID NO: 46 - protein, 7 aa, synthetic construct
  REGION 1..7: note = VL CDR2
QVSKLDP

SEQ ID NO: 47 - protein, 9 aa, synthetic construct
  REGION 1..9: note = VL CDR3
MQGSSLPWT

SEQ ID NO: 48 - protein, 854 aa, synthetic construct
  REGION 1..854: note = C31
EVKFEESGGGLVQPGGSIKLSCAASGVTFSEAWMDWVRQSPEKGLEWVAEIRDKPNSYVTYYAESVKGRFTISRDDSKSRVYLQMNNLRTEDTGIYYCTGAMDYWGQGTSVTVSSGGGGSDIVMTQTPLSLSVTIGQPASISCKSSQSLLYSNGKTYLNWLQQRPGQSPKRLIYQVSKLDPGIPDRFSGSGSETDFTLKISRVEAEDLGIYYCMQGSSLPWTFGGGTKLEIKASSHFVPVFLPAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMEDFNMESDSFEDFWKGEDLSNYSYSSTLPPFLLDAAPCEPESLEINKYFVVIIYALVFLLSLLGNSLVMLVILYSRVGRSVTDVYLLNLALADLLFALTLPIWAASKVNGWIFGTFLCKVVSLLKEVNFYSGILLLACISVDRYLAIVHATRTLTQKRYLVKFICLSIWGLSLLLALPVLLFRRTVYSSNVSPACYEDMGNNTANWRMLLRILPQSFGFIVPLLIMLFCYGFTLRTLFKAHMGQKHRAMRVIFAVVLIFLLCWLPYNLVLLADTLMRTQVIQETCERRNHIDRALDATEILGILHSCLNPLIYAFIGQKFRHGLLKILAIHGLISKDSLPKDSRPSFVGSSSGHTSTTL

SEQ ID NO: 49 - protein, 822 aa, synthetic construct
  REGION 1..822: note = C32
EVKFEESGGGLVQPGGSIKLSCAASGVTFSEAWMDWVRQSPEKGLEWVAEIRDKPNSYVTYYAESVKGRFTISRDDSKSRVYLQMNNLRTEDTGIYYCTGAMDYWGQGTSVTVSSGGGGSDIVMTQTPLSLSVTIGQPASISCKSSQSLLYSNGKTYLNWLQQRPGQSPKRLIYQVSKLDPGIPDRFSGSGSETDFTLKISRVEAEDLGIYYCMQGSSLPWTFGGGTKLEIKASSHFVPVFLPAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 50 - protein, 328 aa, synthetic construct
  REGION 1..328: note = p40
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 51 - protein, 22 aa, synthetic construct
  REGION 1..22: note = P2A
GSGATNFSLLKQAGDVEENPGP

SEQ ID NO: 52 - protein, 21 aa, synthetic construct
  REGION 1..21: note = T2A
GSGEGRGSLLTCGDVEENPGP

SEQ ID NO: 53 - protein, 360 aa, synthetic construct
  REGION 1..360: note = CXCR2
MEDFNMESDSFEDFWKGEDLSNYSYSSTLPPFLLDAAPCEPESLEINKYFVVIIYALVFLLSLLGNSLVMLVILYSRVGRSVTDVYLLNLALADLLFALTLPIWAASKVNGWIFGTFLCKVVSLLKEVNFYSGILLLACISVDRYLAIVHATRTLTQKRYLVKFICLSIWGLSLLLALPVLLFRRTVYSSNVSPACYEDMGNNTANWRMLLRILPQSFGFIVPLLIMLFCYGFTLRTLFKAHMGQKHRAMRVIFAVVLIFLLCWLPYNLVLLADTLMRTQVIQETCERRNHIDRALDATEILGILHSCLNPLIYAFIGQKFRHGLLKILAIHGLISKDSLPKDSRPSFVGSSSGHTSTTL

SEQ ID NO: 54 - protein, 10 aa, synthetic construct
GGGGSGGGGS

SEQ ID NO: 55 - protein, 15 aa, synthetic construct
GGGGSGGGGSGGGGS

SEQ ID NO: 56 - protein, 20 aa, synthetic construct
GGGGSGGGGSGGGGSGGGGS

SEQ ID NO: 57 - protein, 5 aa, synthetic construct
GGGGS

(19) United States
(12) Patent Application Publication
(10) Pub. No.: US 2026/0325638 A1
(43) Pub. Date: Oct. 22, 2026

(54) CD24 BINDING PROTEIN AND USE THEREOF

(71) Applicants: ACROIMMUNE BIOPHARMA CO., LTD., Jiangsu (CN); ACROIMMUNE BIOTECH CO., LTD., Guangdong (CN)

(72) Inventors: Dan TONG, Jiangsu (CN); Yang LIU, Jiangsu (CN)

(73) Assignees: ACROIMMUNE BIOPHARMA CO., LTD., Jiangsu (CN); ACROIMMUNE BIOTECH CO., LTD., Guangdong (CN)

(21) Appl. No.: 19/733,220

(22) Filed: Jul. 7, 2026

Related U.S. Application Data
(63) Continuation-in-part of application No. 19/125,364, filed on Oct. 30, 2023.

(30) Foreign Application Priority Data
Nov. 1, 2022 (WO) PCT/CN2022/128954

(51) Int. Cl. A61K 40/31; A61K 40/11; A61K 40/15; A61K 40/17; A61K 40/30; A61K 40/41; A61P 35/00; C07K 14/705; C07K 16/28

(52) U.S. Cl. CPC A61K 40/31; A61K 40/11; A61K 40/15; A61K 40/17; A61K 40/30; A61K 40/41; A61P 35/00; C07K 14/7051; C07K 14/70517; C07K 14/70521; C07K 16/2896; C07K 2319/03

(57) ABSTRACT

The present application relates to the field of biological medicine, and in particular to a CD24-binding protein and uses thereof.

### CROSS-REFERENCE TO RELATED APPLICATION

This application is a continuation-in-part of U.S. patent application Ser. No. 19/125,364, filed Apr. 29, 2025, which is a U.S. national phase application under 35 U.S.C. § 371 of International Application No. PCT/CN2023/127598, filed Oct. 30, 2023, which claims priority to International Application No. PCT/CN2022/128954, filed Nov. 1, 2022. Each of the foregoing applications is hereby incorporated herein by reference in its entirety.

### REFERENCE TO SEQUENCE LISTING

In accordance with 37 CFR § 1.52(e)(5) and with 37 CFR § 1.831, the specification makes reference to a Sequence Listing submitted electronically as a .xml file named 20260701_272-PA-009US.DIV1_SequenceListing.xml. said .xml copy, created on Jul. 1, 2026 is 80,000 bytes in size. The entire contents of the Sequence Listing are hereby incorporated by reference.

### FIELD OF THE INVENTION

The present application relates to the field of biological medicine, and in particular to a CD24-binding protein and uses thereof.

### BACKGROUND OF THE INVENTION

Immune cells expressing chimeric antigen receptors (CAR) can specifically recognize antigens expressed on the surface of tumor cells and mediated killing and removing tumors. For example, when antigen-positive cells targeted by a CAR are present, cells expressing the CAR can recognize and kill these antigen-positive cells.

However, the existing CAR in the art still has problems such as low activity, weak cytotoxic efficacy of CAR-expressing cells, and problematic in vivo safety. Therefore, it is desirable to further optimize the CAR in the art, in order to achieve the effect of increasing the cytotoxic efficacy of CAR-expressing cells and improve safety.

### SUMMARY OF THE INVENTION

The present application provides an antigen-binding protein with an improved structure, and the antigen-binding protein may be in the form of a chimeric antigen receptor (CAR). The antigen-binding protein may have one or more effects selected from the group consisting of: (1) excellent expression efficiency; (2) significant antitumor activity of cells expressing the antigen-binding protein; (3) sustained antitumor activity of cells expressing the antigen-binding protein; (4) significant cell activation ability of cells expressing the antigen-binding protein; and (5) appropriate cytokine secretion capacity of cells expressing the antigen-binding protein.

In one aspect, the present application provides an antigen-binding protein, wherein the antigen-binding protein binds to CD24, after which cells expressing the antigen-binding protein have an ability to express cytokines at a low level. For example, the antigen-binding fragment comprises a light chain variable region and a heavy chain variable region, with a linker between the light chain variable region and the heavy chain variable region, the linker having a length of 10 amino acids or less.

In another aspect, the present application provides a polypeptide, comprising the antigen-binding protein of the present application.

In another aspect, the present application provides a nucleic acid, encoding the antigen-binding protein of the present application and/or the polypeptide of the present application.

In another aspect, the present application provides a vector, comprising the nucleic acid of the present application.

In another aspect, the present application provides a cell, comprising the antigen-binding protein of the present application, the polypeptide of the present application, the nucleic acid of the present application and/or the vector of the present application.

In another aspect, the present application provides a preparation method for the antigen-binding protein of the present application and/or the polypeptide of the present application, wherein the preparation method comprises culturing the cell of the present application under conditions allowing expression of the antigen-binding protein and/or the polypeptide.

In another aspect, the present application provides a composition, comprising the antigen-binding protein of the present application, the polypeptide of the present application, the nucleic acid of the present application, the vector of the present application, and/or the cell of the present application, and optionally, a pharmaceutically acceptable carrier.

In another aspect, the present application provides a kit, comprising the antigen-binding protein of the present application, the polypeptide of the present application, the nucleic acid of the present application, the vector of the present application, the cell of the present application, and/or the composition of the present application.

In another aspect, the present application provides a method for stimulating an immune response, comprising administering the antigen-binding protein of the present application, the polypeptide of the present application, the nucleic acid of the present application, the vector of the present application, the cell of the present application, the composition of the present application and/or the kit of the present application.

In another aspect, the present application provides a use of the antigen-binding protein of the present application, the polypeptide of the present application, the nucleic acid of the present application, the vector of the present application, the cell of the present application, the composition of the present application and/or the kit of the present application in preparation of a medicament, said medicament is used for preventing, alleviating and/or treating a tumor.

In another aspect, the present application provides a method for preventing, alleviating and/or treating a tumor, comprising administering the antigen-binding protein of the present application, the polypeptide of the present application, the nucleic acid of the present application, the vector of the present application, the cell of the present application, the composition of the present application and/or the kit of the present application.

In another aspect, the present application provides a use of the antigen-binding protein of the present application, the polypeptide of the present application, the nucleic acid of the present application, the vector of the present application, the cell of the present application, the composition of the present application and/or the kit of the present application for preventing, alleviating and/or treating a tumor.

Other aspects and advantages of the present application can be readily perceived by those skilled in the art from the detailed description below. The detailed description below only illustrates and describes the exemplary embodiments of the present application. As would be appreciated by those skilled in the art, the content of the present application enables those killed in the art to change the disclosed specific embodiments without departing from the spirit and scope of the invention involved in the present application. Accordingly, the accompanying drawings and the description in the specification of the present application are merely for an exemplary but not restrictive purpose.

### BRIEF DESCRIPTION OF THE DRAWINGS

The specific features of the invention involved in the present application are listed in the appended claims. The characteristics and advantages of the invention involved in the present application can be better understood by referring to the exemplary embodiments and the accompanying drawings described in detail below. The accompanying drawings are briefly illustrated as follows.

FIG. 1 shows the antitumor activity and cytokine expression levels of H3L3-CAR and PP6373-CAR. C1 was derived by modifying the sequence of H3L3-CAR, C2 was derived by modifying the sequence of PP6373-CAR, and the modified C1 and C2 showed higher anti-tumor activity and cytokine secretion level.

FIG. 2 shows the expression of CAR structures such as C3, C5, C7, C9, C11, C13 and C15; all of these CARs were derived by adjusting the hinge region, transmembrane region and costimulatory domain of the CAR structure on the basis of C1.

FIG. 3 shows the antitumor activity of CAR structures such as C1, C3, C5, C7, C9, C11, C13 and C15 of the present application.

FIG. 4 shows the cytokine secretion of CAR structures such as C1, C3, C5, C7, C9, C11, C13 and C15 of the present application.

FIG. 5 shows the expression of CAR structures such as C4, C6, C8, C10, C12, C14 and C16, all derived by adjusting the hinge region, transmembrane region and costimulatory domain of the CAR structure on the basis of C2.

FIG. 6 shows the antitumor activity of CAR structures such as C2, C4, C6, C8, C10, C12, C14, and C16 of the present application.

FIG. 7 shows the cytokine secretion of CAR structures such as C2, C4, C6, C8, C10, C12, C14 and C16 of the present application.

FIG. 8 shows the expression of C23, C27 and C28, all derived by adjusting the length of a linker or the type of a transmembrane region of the present application.

FIG. 9 shows the in vitro cytotoxic efficacy of C23, C27, and C28, all derived by adjusting the length of a linker or the type of a transmembrane region of the present application.

FIG. 10 shows the cytokine secretion of C23, C27, and C28, all derived by adjusting the length of a linker or the type of a transmembrane region of the present application.

FIG. 11 shows the expression of C28, C29 and C30, all derived by adjusting the length of a linker of the present application.

FIG. 12 shows the in vitro cytotoxic efficacy and cytokine secretion of C28, C29, and C30 derived by adjusting the length of a linker of the present application.

FIG. 13 shows the expression of C31 derived by adding a chemokine receptor CXCR2 of the present application.

FIG. 14 shows the in vitro killing efficacy of C31 derived by adding a chemokine receptor CXCR2 of the present application.

FIG. 15 shows the cytokine secretion of C31 derived by adding a chemokine receptor CXCR2 of the present application.

FIG. 16 shows the in vitro cytotoxic efficacy and cytokine secretion of C32 derived by adding a cytokine IL-12β (p40) of the present application.

FIG. 17 shows the capacity of the CAR structures of C8 and C28 of the present application to clear tumors and their safety results.

FIG. 18 shows the capacity of the CAR structures of C28 and C31 of the present application to reach a tumor site.

FIG. 19 shows the capacity of the CAR structures of C28 and C31 of the present application to remove tumors.

FIG. 20 shows the capacity of the CAR structures of C28, C31 and C32 of the present application to remove tumors and the statistics of their safety results.

FIG. 21 shows a schematic representation of the modular domains in the CAR construct used for CD24 targeting.

FIG. 22 shows that C8 CAR-T cells demonstrate enhanced antitumor efficacy in an ovarian cancer NCG xenograft mouse model. (A) In vivo functional activity of NCG mice were intraperitoneally injected (i.p.) on d-19 with 2×10⁶ SK-OV-3-CD24-lucGFP cells. On d0, animals received intravenous various dose of C8 CAR-T cells (5×10⁶ C8-low; 1×10⁷ C8-medium; 2×10⁷ C8-high) and 2×10⁷ NC cells. (B) Tumor regression monitored by bioluminescent imaging and (C) quantified by total radiance. (D) Fraction survival. Statistical significance was evaluated using two-tailed unpaired t-tests (**P<0.01, ***P<0.001, ****P<0.0001).

FIG. 23 shows that C50 CAR-T cells demonstrate enhanced antitumor efficacy in an ovarian cancer NCG xenograft mouse model. NCG mice were subcutaneously inoculated (s.c.) on d-19 with 2×10⁶ SK-OV-3-CD24-lucGFP cells. On d0, animals received 1×10⁷ intravenous dose of C28 and C50 CAR-T cells. Tumor regression monitored by bioluminescent imaging (A) and quantified by total radiance (B) and Tumor volume (C) measured twice a week using calipers and calculated as (length×width²)/2. C50 CAR-T cells exhibited potent antitumor activity within the effective dose range of 1×10⁶ to 2×10⁷ cells. (D) Bioluminescent imaging, (E) Total radiance (p/sec/cm²/sr), and (F) Tumor volume analysis (mm³).

FIG. 24 shows that C50 CAR-T cells exhibit enhanced antitumor efficacy following intraperitoneal tumor implantation in an ovarian cancer NCG xenograft mouse model. NCG mice were intraperitoneally injected (i.p.) on d-13 with 2×10⁶ SK-OV-3-CD24-lucGFP cells. On d0, the animals received an intravenous dose of 1×10⁷ C28 and C50 CAR-T cells. (A) Tumor regression was monitored by bioluminescent imaging, and (B) tumor burden was quantified by total radiance. Additionally, a dose-response study was performed. NCG mice were intraperitoneally injected (i.p.) on d-13 with 2×10⁶ SK-OV-3-CD24-lucGFP cells. On d0, mice received varying doses of C50 CAR-T cells (3×10⁵, 1×10⁶, 3×10⁶, 1×10⁷) via intravenous injection.

FIG. 25 shows in vivo pharmacokinetic analysis of C50 CAR-T cells in an ovarian cancer xenograft model. Tumor volumes were measured with calipers twice a week following CAR-T cell infusion. (A) Tumor growth curves in treated mice. Peripheral blood was analyzed by flow cytometry for total CAR-T and CD3⁺ T-cell number on days 1, 3, 5, 7, 14, 21, 28, and 39 days post-infusions. (B) Kinetics of CD3⁺ T-cell expression in peripheral blood. (C) Kinetics of CAR-T cell expression in peripheral blood. Statistical analysis was performed using a one-tailed, unpaired t-test (**p<0.01; ***p<0.001; ****p<0.0001; ns, not significant).

FIG. 26 shows that C50 CAR-T cells demonstrate enhanced antitumor efficacy in a glioblastoma xenograft mouse model following either systemic or intracerebral administration. In vivo antitumor efficacy in C-NKG mice: animals were intracerebral inoculated (i.e.) on Day0 (d-12) with 3×10⁵ U87-CD24-lucGFP cells and treated i.e. or i.v. on d0 with C50 CAR-transduced T cells. (A) Tumor regression monitored by bioluminescent imaging and (B) quantified by total radiance (p/sec/cm²/sr).

FIG. 27 shows that C50 CAR-T cells demonstrate enhanced antitumor efficacy in mantle cell lymphoma and acute lymphoblastic leukemia xenograft mouse models. (A-B) In vivo antitumor efficacy in C-NKG mice: animals were intravenously inoculated (i.v.) on Day0 (d-9) with 2×10⁶ JeKo-1-lucGFP cells and treated intravenously on d0 with 5×10⁶ C50 CAR-transduced T cells. (A) Tumor regression monitored by bioluminescent imaging and (B) quantified by total radiance (p/sec/cm²/sr). (C-E) In vivo antitumor efficacy in NCG mice: animals were intravenously inoculated (i.v.) on Day0 (d-8) with 2×10⁶ Nalm-6-lucGFP cells and treated intravenously on d0 with 5×10⁶ C50 CAR-transduced T cells. (C) Tumor regression monitored by bioluminescent imaging and (D) quantified by total radiance (p/sec/cm²/sr). (E) Kaplan-Meier survival curves presenting the overall survival of each group. The log-rank Mantel-Cox test was used to analyze the survival of each group. (F-G) In vivo antitumor efficacy in C-NKG mice: animals were intravenously inoculated (i.v.) on Day0 (d-9) with 2×10⁶ JeKo-1-lucGFP cells and treated intravenously on d0 with various dose of C50 CAR-T cells (1×10⁶, 3×10⁶, 5×10⁶, 1×10⁷, 2×10⁷). (F) Bioluminescent imaging and (G) quantified by total radiance (p/sec/cm²/sr).

FIG. 28 shows biodistribution of C50 CAR-T cells and T-cell markers in mouse tissues. Flow cytometric analysis was performed on tumor and multiple organ samples from SK-OV-3-CD24-lucGFP ovarian cancer xenograft mice within 28 days post-infusion to assess the distribution of C50 CAR-T cells and T-cell markers (CD3⁺ and CD45⁺). C50 CAR-T cell biodistribution: (A) PBL, (B) spleen, (C) tumor, (D) liver, (E) lung, (F) heart, and (G) kidney. CD3⁺ T-cell biodistribution: (H) PBL, (I) spleen, (J) tumor, (K) liver, (L) lung, (M) heart, and (N) kidney. CD45⁺ T-cell biodistribution: (O) liver, (P) lung, (Q) heart, and (R) kidney. Tumor cells antigen expression: (S) CD24 expression in SK-OV-3-CD24-lucGFP tumor cells FIG. 29 shows general toxicity assessment of C50 CAR-T cells in a mantle cell lymphoma (MCL) xenograft model. Peripheral blood was collected on Days 0, 1, 4, 7, 11, 14, 21, 24, 28, and 42 following C50 CAR-T infusion. Human- and mouse-derived inflammatory cytokines in serum were tested by flow cytometry. Human cytokine profiles were determined using the LEGENDplex™ Human Inflammation Panel 1 (13-plex), measuring (A) IFN-γ, (B) IL-10, (C) TNF-α, (D) IL-17A, (E) IL-18, (F) IL-23, (G) IL-8, (H) MCP-1, (I) IL-6, (J) IL-12p70, (K) IFN-α2, (L) IL-1β, and (M) IL-33. Mouse cytokines were analyzed using the LEGENDplex™ Mouse Inflammation Panel (13-plex), measuring (N) MCP-1, (O) IL-6, (P) IL-27, (Q) IL-23, (R) IL-10, (S) IL-la, (T) TNF-α, (U) IL-17A, (V) IL-1β, (W) IFN-β, (X) IFN-γ, (Y) IL-12p70, and (Z) GM-CSF.

### DETAILED DESCRIPTION OF THE PREFERRED EMBODIMENTS

The embodiments of the invention of the present application will be illustrated by specific examples below. Those familiar with this technology can readily understand other advantages and effects of the invention of the present application from the content disclosed in the specification.

In the present application, the term “CD24” generally refers to a CD24 protein or a gene encoding the same. CD24 can be found under UniProt Number P25063. This term comprises its variants, homologs, and functionally active fragments.

In the present application, the term “antigen-binding protein” generally refers to a protein capable of binding to one or more antigens, or functionally active fragments thereof. For example, an exemplary antigen-binding protein comprises a chimeric antigen receptor and a variety of other molecules that are known in the art and comprise domains capable of recognizing antigens.

In the present application, the term “chimeric antigen receptor (CAR)” generally refers to a fusion protein comprising an extracellular domain capable of binding to an antigen, and at least one transmembrane domain. CAR can be the core component of a CAR cell and can comprise an antigen (e.g., a tumor-specific and/or tumor-associated antigen)-binding domain, a hinge domain, a transmembrane domain and an intracellular domain.

In the present application, the term “signal peptide” generally refers to a propeptide that is present as an N-terminal peptide on the precursor form of a protein. The signal peptide functions to facilitate the translocation of an expression polypeptide attached to the endoplasmic reticulum and to express the protein of interest on the cell membrane, during which the signal peptide can generally be cleaved. For example, without the signal peptide, the biological activity of the antigen-binding protein of the present application is not affected as well. For an organism for producing polypeptides, the signal peptide can be heterologous or homologous.

In the present application, the term “antigen-binding domain” generally refers to (specifically) binding to a given target epitope or site on a target molecule (antigen), or interacting with the given target epitope or site, or identifying a domain of the given target epitope or site.

In the present application, the term “specific binding” generally refers to a measurable and/or reproducible interaction, such as the binding between a target and an antibody, and the existence of the target can be determined in a heterogeneous population of molecules (including biomolecules). For example, an antibody specifically binding to a target (which may be an epitope) is an antibody that binds to the target with greater affinity, avidity, easiness, and/or duration than it binds to other targets. In some embodiments, the antibody specifically binds to an epitope on a protein, the epitope being conserved among proteins of different species. In another embodiment, the specific binding may include, but not necessarily, exclusive binding.

In the present application, the term “antibody” generally refers to an immunoglobulin or a fragment or derivative thereof, and encompasses any polypeptide, regardless of in vitro or in vivo, that includes an antigen-binding site. This term includes, but is not limited to, polyclonal, monoclonal, monospecific, multispecific, non-specific, humanized, single-chain, chimeric, synthetic, recombinant, hybrid, mutant and grafted antibodies. The term “antibody” may also comprise antibody fragments, such as Fab, F(ab′)₂, Fv, scFv, Fd, dAb, and other antibody fragments that maintain the antigen-binding function.

In the present application, the term “single-chain antibody (scFv)” may be an antibody formed by linking the heavy chain variable region and the light chain variable region of the antibody via a linker.

In the present application, the term “hinge domain” generally refers to a linkage region between the antigen-binding domain and the transmembrane region. In general, the hinge domain has some degree of flexibility, and the CAR molecule may optionally comprise or not comprise the hinge domain.

In the present application, the term “transmembrane domain” generally refers to the domain in the CAR that crosses the cell membrane, links to the intracellular signaling domain and serves to transmit signals.

In the present application, the term “CD8a” generally refers to the T-cell surface glycoprotein CD8 alpha chain. CD8a can be found under UniProt Number P01732. This term comprises its variants, homologs, and functionally active fragments. In the present application, the term “CD28” generally refers to T-cell-specific surface glycoprotein CD28. CD28 can be found under UniProt Number P10747. This term comprises its variants, homologs, and functionally active fragments.

In the present application, the term “intracellular domain” generally refers to the intracellular fraction of a molecule. The intracellular domain produces signals that promote the immune effector functions of CAR-containing cells. For example, cell lysis activity and auxiliary activity are involved. For example, the intracellular domain may comprise only a truncated fraction that preserves the intracellular signaling function.

In the present application, the term “costimulatory signaling domain” generally refers to an intracellular domain that can provide immune costimulatory molecules. In the present application, the term “costimulatory” generally refers to the source of lymphocyte activation signals, which are typically produced by the interaction of costimulatory molecules and their receptors on the surface of immune cells involved in adaptive immunity (between T cells and B cells or between antigen-presenting cells and T cells).

In the present application, the term “signaling domain” generally refers to a domain that is located inside a cell and capable of conducting signals. In the present application, the intracellular signaling domain may transmit signals into a cell.

In the present application, the term “CD3ζ” generally refers to CD247 or T-cell surface glycoprotein CD3 zeta chain. CD3ζ can be found under UniProt Number P20963. This term comprises its variants, homologs, and functionally active fragments.

For example, the sequence mentioned in the present application comprises a sequence that is at least approximately 95% homologous to this sequence. For example, the sequence mentioned in the present application comprises a sequence that is at least approximately 95%, 96%, 97%, 98% or 99% homologous to this sequence.

In the present application, the term “immune cells” generally refers to cells involved in an immune response, for example, those promoting an immune effector response. Examples of the immune cells comprise, but are not limited to, T cells, B cells, natural killer (NK) cells, NKT cells, mast cells, granulocytes, monocytes, lymphocytes, and macrophages. This term also includes engineered immune cells, such as immune cells that have been genetically modified by adding exogenous genetic materials in the form of DNA or RNA to total genetic materials of the cells.

In the present application, the term “cytotoxic efficacy” refers to the killing of cells by exposing these cells to an effective amount of antibodies, immunoconjugates, bispecific/multispecific molecules, or their combinations. The method may comprise killing antigen expression-positive cells, optionally in the presence of effector cells, for example, by CDC, apoptosis, ADCC, ADCP, phagocytosis, or by a combination of two or more of these mechanisms.

In the present application, the term “directly or indirectly linked to” refers to direct linkage by peptide bonds, or indirect linkage by linkers or by non-peptide linkage.

In the present application, the term “proliferation” refers to an increase in cell division (symmetrical or asymmetric division of cells). “Proliferation” may refer to the symmetrical or asymmetric division of T cells. An “increase in proliferation” occurs when the count of cells in a treated sample increases as compared with cells in an untreated sample.

In the present application, the term “subject” includes any human or non-human animal. The term “non-human animals” comprise all vertebrates, for example, mammals and non-mammals, such as non-human primates, sheep, dogs, cats, cows, horses, chickens, amphibians and reptiles. The non-human animals may be mammals, such as non-human primates, sheep, dogs, cats, cows and horses.

In the present application, the term “therapeutically effective dose” refers to the dose of the antibody of the present application that is sufficient to prevent or alleviate symptoms associated with a disease or disorder (e.g., cancer). The therapeutically effective dose is associated with the disease being treated, and those skilled in the art can easily determine the actual effective dose.

In the present application, the term “medicament” generally refers to a chemical compound or composition that is capable of inducing the desired therapeutic effect when administered correctly to a patient.

In the present application, the term “composition” refers to a mixture comprising one or more of the compounds of the present application, or a physiologically/pharmaceutically acceptable salt or pro-drug thereof, and other chemical components, for example physiological/pharmaceutically acceptable carriers and excipients. The pharmaceutical composition is intended to promote the administration to an organism, facilitating the absorption of an active ingredient to thereby exert a biological activity. A therapeutic composition should generally be sterile and remain stable under manufacturing and storage conditions. The composition may be formulated as a solution, a microemulsion, a dispersant, a liposome, or other ordered structures suitable for a high antibody concentration. A sterile injectable solution may be prepared by incorporating a required amount of active compound (i.e., the antibody or antibody moiety) together with one or a combination of the components listed above into an appropriate solvent, followed by filtration and sterilization as required.

In the present application, the term “vector” generally refers to a nucleic acid molecule capable of transferring another nucleic acid to which it is attached. One type of vector is a “plasmid”, which refers to a circular double-stranded DNA loop into which other DNA segments can be linked. Another type of vector is a viral vector, in which other DNA segments may be linked into a viral genome. Some vectors are capable of autonomously replicating in host cells into which they are introduced into (e.g., bacterial vectors with origins of bacterial replication and episomal mammalian vectors). Other vectors (e.g., non-episomal mammalian vectors) may be integrated into the genomes of host cells when introduced into the host cells and thus replicate together with the host genomes. These vectors include, for example, naked RNA polynucleotides incapable of replicating autonomously, naked DNA polynucleotides, polynucleotides composed of DNA and RNA in the same strand, poly-lysine-conjugated DNA or RNA, peptide-conjugated DNA or RNA, liposome-conjugated DNA, etc. Furthermore, some vectors are capable of directing the expression of genes to which these vectors are effectively linked. Such vectors are referred to as “recombinant expression vectors” (or simply “expression vectors”) in the present application. In general, the expression vectors used in recombinant DNA technologies are usually in the form of plasmids. In the present specification, the terms “plasmid” and “vector” are used interchangeably, as the plasmid is the most commonly used form of vector.

In the present application, the term “adjuvant” generally refers to any substance that assists or regulates the action of a medicament. It includes, but not is limited to, an immunological adjuvant, which enhances or diversifies the immune response to an antigen.

In the present application, the term “tumor” or “tumor cell” generally refers to or describes a physiological condition in mammals that is usually characterized by unregulated cell growth. Examples of tumors include, but are not limited to, carcinomas, lymphomas, blastomas (including medulloblastoma and retinoblastoma), sarcomas (including liposarcoma and synovial cell sarcoma), neuroendocrine tumors (including carcinoid tumors, gastrinomas, and islet-cell carcinomas), mesotheliomas, schwannomas (including acoustic neuromas), meningiomas, adenocarcinomas, and melanomas. The “tumor cell” may further include a “solid tumor”, which refers to a tumor selected from the group consisting of: gastrointestinal cancer, pancreatic cancer, glioblastoma, cervical cancer, ovarian cancer, bladder cancer, hepatoma, breast cancer, colon cancer, rectal cancer, colorectal cancer, endometrial or uterine cancer, salivary gland cancer, kidney cancer, prostate cancer, vulvar cancer, thyroid cancer, hepatic carcinoma, anal cancer, penile cancer, testicular cancer, esophageal cancer, bile duct tumor, and head and neck cancer.

In the present application, the term “antigen-positive tumor” refers to tumor cells that express antigenic proteins on their surface. For the purpose of determining whether cells express antigenic proteins on their surface, antigen mRNA expression is considered to be related to antigenic protein expression on the cell surface, and the antigen mRNA expression may be determined by a method selected from in situ hybridization and RT-PCR (including quantitative RT-PCR). Or, for example, antibodies against antigenic proteins may be used to determine the antigenic protein expression on the cell surface in methods such as immunohistochemistry and FACS. For example, an antigen-positive tumor may be a mammalian implanted tumor. In the present application, the term “antigen-positive tumor cell” refers to a cell expressing antigenic proteins on it surface.

In the present application, the term “CD24-positive malignancy” generally refers to any neoplastic disease, cancer, tumor, or malignant condition characterized by the abnormal expression, over-expression, or cell-surface presentation of Cluster of Differentiation 24 (CD24) protein or its corresponding mRNA in a subset or the entirety of the tumor cells or tumor-associated stroma. Consistently with the determination of antigen-positive tumors described herein, a malignancy may be classified as a “CD24-positive malignancy” if CD24 expression is confirmed at the transcriptional level (e.g., via in situ hybridization, RT-PCR, or quantitative RT-PCR) or at the translational level (e.g., via immunohistochemistry (IHC), Western blotting, or fluorescence-activated cell sorting (FACS) using anti-CD24 antibodies). The term encompasses both solid tumors and hematological malignancies. In some embodiments, the CD24-positive malignancy includes, but is not limited to, ovarian cancer, brain cancer (such as glioblastoma, astrocytoma, or medulloblastoma), breast cancer, colorectal cancer, gastric cancer, pancreatic cancer, lung cancer (including non-small cell lung cancer), liver cancer, prostate cancer, bladder cancer, as well as hematological malignancies such as mantle cell lymphoma (MCL), acute lymphoblastic leukemia (ALL), chronic lymphocytic leukemia (CLL), and multiple myeloma. In some embodiments, the term also encompasses recurrent, metastatic, or relapsed and refractory (R/R) forms of these CD24-expressing cancers, as well as mammalian implanted tumor models thereof.

### DETAILED DESCRIPTION OF THE INVENTION

### Chimeric Antigen Receptor

In one aspect, the present application provides a group of improved antigen-binding protein G1, wherein the G1 binds to CD24, and the enhanced effect of cells expressing said G1 on the killing ability of CD24-positive tumor cells, or the ability of cells expressing said G1 to express cytokines after being stimulated by CD24-positive tumor cells, is higher than that of cells expressing the existing CD24 antigen-binding proteins. For example, approximately 5%, 10%, 20%, 30%, 40%, 50%, 70%, 100%, 150%, 200%, 300%, 400%, 500%, 4000%, 5000% or 5000% higher than cells expressing CD24 antigen-binding proteins that have been disclosed in the art.

In one aspect, the present application provides a group of further improved antigen-binding protein G2 based on the G1, wherein the G2 binds to CD24, G2-expressing cells maintain a capacity to kill the CD24-positive tumor cells, and an ability of expressing cytokines after stimulation by the CD24-positive tumor cells is at least about 50% lower than that of cells expressing G1. For example, at least approximately 5%, 10%, 20%, 30%, 40% or 50% lower than G1-expressing cells.

In one aspect, the present application provides a group of further improved antigen-binding protein G3 based on the G1 or G2 for use in expression in cells, wherein the G3-expressing cells also express chemokine receptors. For example, the chemokine receptors may be chemokine receptors known in the art. For example, the chemokine receptors may comprise CXCR2 or a functional fragment thereof. For example, the CXCR2 may comprise an amino acid sequence as set forth in SEQ ID NO: 53.

In one aspect, the present application provides a group of further improved antigen-binding protein G3 based on the G1 or the G2 for use in expression in cells, wherein the G3-expressing cells also express cytokines. For example, the cytokines may comprise IL-12β or a functional fragment thereof. For example, the IL-12β may comprise an amino acid sequence as set forth in SEQ ID NO: 50. In one aspect, the present application provides an antigen-binding protein, wherein the antigen-binding protein binds to CD24, after which cells expressing the antigen-binding protein have an ability to express cytokines at a low level.

For example, the cytokines comprise, but are not limited to, IFN-γ, IL-6, TNF-α, and/or IL-2.

For example, in terms of the antigen-binding protein as described, compared with the corresponding cells that do not express the antigen-binding protein of the present application, the cells expressing the antigen-binding protein have a cytokine expression level of approximately 2000 pg/ml or less in the co-culture supernatant of these cells and tumor cells for about 24 h. For example, compared with the corresponding cells that do not express the antigen-binding protein of the present application, the cells expressing the antigen-binding protein have a cytokine expression level of approximately 2000 pg/ml or less, approximately 1000 pg/ml or less, approximately 500 pg/ml or less, approximately 200 pg/ml or less, approximately 100 pg/ml or less, or approximately 50 pg/ml or less in the co-culture supernatant of these cells and tumor cells for about 24 h.

For example, the antigen-binding protein comprises an antigen-binding domain. For example, the antigen-binding domain is capable of recognizing an antibody to the antigen or an antigen-binding fragment thereof. For example, the antigen-binding fragment comprises a single-chain antibody. For example, the antigen-binding fragment comprises a heavy chain variable region. For example, the heavy chain variable region comprises a sequence as set forth in SEQ ID NO: 23 or SEQ ID NO: 24. For example, the heavy chain variable region comprises HCDRs 1 to 3. For example, the heavy chain variable region comprises HCDRs 1 to 3 as set forth in SEQ ID Nos: 42 to 44. For example, the antigen-binding fragment comprises a light chain variable region. For example, the light chain variable region comprises a sequence as set forth in SEQ ID NO: 25 or SEQ ID NO: 26. For example, the light chain variable region comprises LCDRs 1 to 3. For example, the light chain variable region comprises LCDRs 1 to 3 as set forth in SEQ ID Nos: 45 to 47.

For example, the antigen-binding fragment comprises a light chain variable region and a heavy chain variable region. For example, the light chain variable region comprises a sequence as set forth in SEQ ID NO: 25 or SEQ ID NO: 26, and the heavy chain variable region comprises a sequence as set forth in SEQ ID NO: 23 or SEQ ID NO: 24. For example, a linker is comprised between the light chain variable region and the heavy chain variable region. For example, the linker has a length of 1, 2, 3, 4, 5, 6, 7, 8, 9, 10, or more amino acids. For example, the linker comprises an alanine-serine polymer or other flexible peptide sequences known in the art. For example, the linker comprises amino acid substitutions. For example, the linker comprises a sequence of 1 or more GGGGSs (as set forth in SEQ ID NO: 27), such as 1, 2, 3, 4, or 5 GGGGSs; for example, the linker comprises a sequence of 1 or more Gs (as set forth in SEQ ID NO: 28), such as 1, 2, 3, 4, 5, 6, 7, 8, 9, or 10 Gs; for example, the linker comprises a sequence of one or more GSs (SEQ ID NO: 29), such as 1, 2, 3, 4, or 5 GSs; for example, the linker comprises a sequence of one or more GGGSs (as set forth in SEQ ID NO: 30), such as 1, 2 or, 3, 4, or 5 GGGSs; and for example, the linker comprises a sequence of one or more GSGGSs (SEQ ID NO: 31), such as 1 or 2 GSGGSs.

For example, the antigen-binding protein further comprises a hinge domain, which is directly or indirectly linked to the antigen-binding domain. For example, the hinge domain comprises a hinge domain structure known in the art for chimeric antigen receptors. For example, the hinge domain comprises, but is not limited to, a hinge domain of CD8 or CD28, or of a functional fragment thereof. For example, the hinge domain comprises a sequence as set forth in SEQ ID NO: 32 or 33.

For example, the antigen-binding protein further comprises a transmembrane domain, which is directly or indirectly linked to the hinge domain. For example, the transmembrane domain of the present application comprises an intracellular domain structure known in the art for chimeric antigen receptors. For example, the transmembrane domain comprises, but is not limited to, a transmembrane domain of CD8 or CD28, or of a functional fragment thereof. For example, the transmembrane domain comprises a sequence as set forth in SEQ ID NO: 34 or 35.

For example, the antigen-binding protein further comprises an intracellular domain, which is directly or indirectly linked to the transmembrane domain. For example, the intracellular domain of the present application comprises an intracellular domain structure known in the art for chimeric antigen receptors. For example, the antigen-binding protein comprises one or more intracellular domains. For example, the antigen-binding protein comprises 1, 2, 3, 4, or 5 of the additional intracellular domains.

For example, the intracellular domain may comprise a costimulatory signal domain and/or a signaling domain. For example, the intracellular domain may comprise 1, 2, 3, 4, or 5 of the costimulatory signal domains. For example, the intracellular domain may comprise 1 of the costimulatory signal domains. For example, the costimulatory domain comprises, but is not limited to, the costimulatory domain of 41-BB, CD28, or OX40, or functional fragments thereof. For example, the costimulatory domain comprises a sequence as set forth in SEQ ID NO: 36, SEQ ID NO: 37 or SEQ ID NO: 38. For example, the costimulatory domain comprises a costimulatory domain of 41-BB, CD28 or a functional fragment thereof. For example, the costimulatory domain comprises sequences as set forth in SEQ ID NO: 36 and SEQ ID NO: 37. For example, the costimulatory domain comprises a costimulatory domain of 41-BB or a functional fragment thereof. For example, the costimulatory domain comprises a sequence as set forth in SEQ ID NO: 36. For example, the costimulatory domain comprises a costimulatory domain of CD28 or a functional fragment thereof. For example, the costimulatory domain comprises a sequence as set forth in SEQ ID NO: 37. For example, the costimulatory domain comprises a costimulatory domain of OX40 or a functional fragment thereof. For example, the costimulatory domain comprises a sequence as set forth in SEQ ID NO: 38. For example, the intracellular domain may comprise 1, 2, 3, 4, or 5 of the signaling domains. For example, the signaling domain comprises, but is not limited to, a signaling domain of CD3ζ or a functional fragment thereof. For example, the signaling domain comprises a sequence as set forth in SEQ ID NO: 39.

For example, the antigen-binding protein comprises a chimeric antigen receptor.

For example, the chimeric antigen receptor may comprise an antigen-binding domain, a hinge domain, a transmembrane domain, and/or an intracellular domain in sequence from the N-terminal to the C-terminal.

For example, the chimeric antigen receptor of the present application comprises an H3L3 heavy chain variable region as set forth in SEQ ID NO: 23, an H3L3 light chain variable region as set forth in SEQ ID NO: 25, a CD28 hinge domain as set forth in SEQ ID NO: 32, a CD28 transmembrane domain as set forth in SEQ ID NO: 34, a CD28 intracellular costimulatory domain as set forth in SEQ ID NO: 37, a 41-BB intracellular costimulatory domain as set forth in SEQ ID NO: 36, and a CD3ζ intracellular signaling domain as set forth in SEQ ID NO: 39.

For example, the chimeric antigen receptor of the present application comprises an H3L3 heavy chain variable region as set forth in SEQ ID NO: 23, an H3L3 light chain variable region as set forth in SEQ ID NO: 25, a CD8 hinge domain as set forth in SEQ ID NO: 33, a CD28 transmembrane domain as set forth in SEQ ID NO: 34, a CD28 intracellular costimulatory domain as set forth in SEQ ID NO: 37, a 41-BB intracellular costimulatory domain as set forth in SEQ ID NO: 36, and a CD3ζ intracellular signaling domain as set forth in SEQ ID NO: 39.

For example, the chimeric antigen receptor of the present application comprises an H3L3 heavy chain variable region as set forth in SEQ ID NO: 23, an H3L3 light chain variable region as set forth in SEQ ID NO: 25, a CD28 hinge domain as set forth in SEQ ID NO: 32, a CD28 transmembrane domain as set forth in SEQ ID NO: 34, a CD28 intracellular costimulatory domain as set forth in SEQ ID NO: 37, and a CD3ζ intracellular signaling domain as set forth in SEQ ID NO: 39.

For example, the chimeric antigen receptor of the present application comprises an H3L3 heavy chain variable region as set forth in SEQ ID NO: 23, an H3L3 light chain variable region as set forth in SEQ ID NO: 25, a CD8 hinge domain as set forth in SEQ ID NO: 33, a CD28 transmembrane domain as set forth in SEQ ID NO: 34, a CD28 intracellular costimulatory domain as set forth in SEQ ID NO: 37, and a CD3ζ intracellular signaling domain as set forth in SEQ ID NO: 39.

For example, the chimeric antigen receptor of the present application comprises an H3L3 heavy chain variable region as set forth in SEQ ID NO: 23, an H3L3 light chain variable region as set forth in SEQ ID NO: 25, a CD28 hinge domain as set forth in SEQ ID NO: 32, a CD28 transmembrane domain as set forth in SEQ ID NO: 34, a 41BB intracellular costimulatory domain as set forth in SEQ ID NO: 36, and a CD3ζ intracellular signaling domain as set forth in SEQ ID NO:39.

For example, the chimeric antigen receptor of the present application comprises an H3L3 heavy chain variable region as set forth in SEQ ID NO: 23, an H3L3 light chain variable region as set forth in SEQ ID NO: 25, a CD8 hinge domain as set forth in SEQ ID NO: 33, a CD28 transmembrane domain as set forth in SEQ ID NO: 34, a 41BB intracellular costimulatory domain as set forth in SEQ ID NO: 36, and a CD3ζ intracellular signaling domain as set forth in SEQ ID NO:39.

For example, the chimeric antigen receptor of the present application comprises an H3L3 heavy chain variable region as set forth in SEQ ID NO: 23, an H3L3 light chain variable region as set forth in SEQ ID NO: 25, a CD28 hinge domain as set forth in SEQ ID NO: 32, a CD28 transmembrane domain as set forth in SEQ ID NO: 34, an OX40 intracellular costimulatory domain as set forth in SEQ ID NO: 38, and a CD3ζ intracellular signaling domain as set forth in SEQ ID NO:39.

For example, the chimeric antigen receptor of the present application comprises an H3L3 heavy chain variable region as set forth in SEQ ID NO: 23, an H3L3 light chain variable region as set forth in SEQ ID NO: 25, a CD8 hinge domain as set forth in SEQ ID NO: 33, a CD28 transmembrane domain as set forth in SEQ ID NO: 34, an OX40 intracellular costimulatory domain as set forth in SEQ ID NO: 38, and a CD3ζ intracellular signaling domain as set forth in SEQ ID NO: 39.

For example, the chimeric antigen receptor of the present application comprises a PP6373 heavy chain variable region as set forth in SEQ ID NO: 24, a PP6373 light chain variable region as set forth in SEQ ID NO: 26, a CD28 hinge domain as set forth in SEQ ID NO: 32, a CD28 transmembrane domain as set forth in SEQ ID NO: 34, a CD28 intracellular costimulatory domain as set forth in SEQ ID NO: 37, a 41-BB intracellular costimulatory domain as set forth in SEQ ID NO: 36, and a CD3ζ intracellular signaling domain as set forth in SEQ ID NO: 39.

For example, the chimeric antigen receptor of the present application comprises a PP6373 heavy chain variable region as set forth in SEQ ID NO: 24, a PP6373 light chain variable region as set forth in SEQ ID NO: 26, a CD8 hinge domain as set forth in SEQ ID NO: 33, a CD28 transmembrane domain as set forth in SEQ ID NO: 34, a CD28 intracellular costimulatory domain as set forth in SEQ ID NO: 37, a 41-BB intracellular costimulatory domain as set forth in SEQ ID NO: 36, and a CD3ζ intracellular signaling domain as set forth in SEQ ID NO: 39.

For example, the chimeric antigen receptor of the present application comprises a PP6373 heavy chain variable region as set forth in SEQ ID NO: 24, a PP6373 light chain variable region as set forth in SEQ ID NO: 26, a CD28 hinge domain as set forth in SEQ ID NO: 32, a CD28 transmembrane domain as set forth in SEQ ID NO: 34, a CD28 intracellular costimulatory domain as set forth in SEQ ID NO: 37, and a CD3ζ intracellular signaling domain as set forth in SEQ ID NO: 39.

For example, the chimeric antigen receptor of the present application comprises a PP6373 heavy chain variable region as set forth in SEQ ID NO: 24, a PP6373 light chain variable region as set forth in SEQ ID NO: 26, a CD8 hinge domain as set forth in SEQ ID NO: 33, a CD28 transmembrane domain as set forth in SEQ ID NO: 34, a CD28 intracellular costimulatory domain as set forth in SEQ ID NO: 37, and a CD3ζ intracellular signaling domain as set forth in SEQ ID NO: 39.

For example, the chimeric antigen receptor of the present application comprises a PP6373 heavy chain variable region as set forth in SEQ ID NO: 24, a PP6373 light chain variable region as set forth in SEQ ID NO: 26, a CD28 hinge domain as set forth in SEQ ID NO: 32, a CD28 transmembrane domain as set forth in SEQ ID NO: 34, a 41-BB intracellular costimulatory domain as set forth in SEQ ID NO: 36, and a CD3ζ intracellular signaling domain as set forth in SEQ ID NO: 39.

For example, the chimeric antigen receptor of the present application comprises a PP6373 heavy chain variable region as set forth in SEQ ID NO: 24, a PP6373 light chain variable region as set forth in SEQ ID NO: 26, a CD8 hinge domain as set forth in SEQ ID NO: 33, a CD28 transmembrane domain as set forth in SEQ ID NO: 34, a 41-BB intracellular costimulatory domain as set forth in SEQ ID NO: 36, and a CD3ζ intracellular signaling domain as set forth in SEQ ID NO: 39.

For example, the chimeric antigen receptor of the present application comprises a PP6373 heavy chain variable region as set forth in SEQ ID NO: 24, a PP6373 light chain variable region as set forth in SEQ ID NO: 26, a CD28 hinge domain as set forth in SEQ ID NO: 32, a CD28 transmembrane domain as set forth in SEQ ID NO: 34, an OX40 intracellular costimulatory domain as set forth in SEQ ID NO: 38, and a CD3ζ intracellular signaling domain as set forth in SEQ ID NO:39.

For example, the chimeric antigen receptor of the present application comprises a PP6373 heavy chain variable region as set forth in SEQ ID NO: 24, a PP6373 light chain variable region as set forth in SEQ ID NO: 26, a CD8 hinge domain as set forth in SEQ ID NO: 33, a CD28 transmembrane domain as set forth in SEQ ID NO: 34, an OX40 intracellular costimulatory domain as set forth in SEQ ID NO: 38, and a CD3ζ intracellular signaling domain as set forth in SEQ ID NO:39.

For example, the chimeric antigen receptor of the present application may also be linked to a chemokine receptor via a self-cleaving peptide at the C-terminal of the CD3ζ intracellular signaling domain. For another example, the chimeric antigen receptor of the present application may also be linked to a cytokine via a self-cleaving peptide at the C-terminal of the CD3ζ intracellular signaling domain. For example, the linkage may be down via a 2A peptide. An exemplary 2A peptide may be P2A or T2A, with sequences as set forth in SEQ ID NO: 51 and SEQ ID NO: 52.

For example, the chemokine receptor may be CXCR2 or a functional fragment thereof. The exemplary amino acid sequence of CXCR2 may be as set forth in SEQ ID NO: 53.

For example, the cytokine may be IL-12β or a functional fragment thereof. The exemplary amino acid sequence of IL-12β may be as set forth in SEQ ID NO: 50.

For example, the chimeric antigen receptor of the present application comprises: (a) a CD24-binding domain comprising a VH and a VL, wherein the VH comprises an amino acid sequence as set forth in SEQ ID NO: 24, and the VL comprises an amino acid sequence as set forth in SEQ ID NO: 26, wherein the VH and the VL are linked by a linker comprising an amino acid sequence of SEQ ID NO: 57; (b) a hinge region comprising an amino acid sequence of SEQ ID NO: 33; (c) a transmembrane domain comprising an amino acid sequence of SEQ ID NO: 34; (d) a co-stimulatory domain comprising an amino acid sequence of SEQ ID NO: 37; (e) an CD3ζ signaling domain comprising an amino acid sequence of SEQ ID NO: 39; and (f) a CXCR2 co-expressing with the CAR, wherein the CXCR2 comprises an amino acid sequence of SEQ ID NO: 53.

For example, the improved antigen-binding protein of the present application comprises an amino acid sequence as set forth in any one of SEQ ID NOs: 1 to 21, 48 and 49.

For example, the antigen-binding protein comprises a chimeric antigen receptor.

For example, the chimeric antigen receptor of the present application may be expressed on the surface of an immune cell. For example, the immune cell of the present application may comprise an immune effector cell. For example, the immune cell of the present application may comprise a T cell, a NK cell, and a mixture of the above cells. For example, the immune cell of the present application may comprise one or more chimeric antigen receptors of the present application.

In another aspect, the present application provides a polypeptide, which may comprise the antigen-binding protein of the present application. For example, the polypeptide of the present application may further comprise one or more types of full-length cytokines or fragments thereof.

In another aspect, the present application provides a nucleic acid, which may encode the antigen-binding protein of the present application and/or the polypeptide of the present application.

In another aspect, the present application provides a vector, which may comprise the nucleic acid of the present application.

In another aspect, the present application provides a cell, which may comprise the antigen-binding protein of the present application, the polypeptide of the present application, the nucleic acid of the present application, and/or the vector of the present application. For example, the cell may comprise an immune cell. For example, the cells may comprise a NK cell and/or a T cell.

In another aspect, the present application provides a preparation method for the antigen-binding protein of the present application and/or the polypeptide of the present application, wherein the preparation method may comprise culturing the cell of the present application under conditions allowing expression of the antigen-binding protein and/or the polypeptide.

In another aspect, the present application provides a composition, which may comprise the antigen-binding protein of the present application, the polypeptide of the present application, the nucleic acid of the present application, the vector of the present application, and/or the cell of the present application, and optionally a pharmaceutically acceptable carrier. For example, the pharmaceutically acceptable carrier may comprise any and all of solvents, dispersion media, isotonic agents, and absorption retarders, that are compatible with immune effector cells. Such carriers are generally safe and non-toxic.

In another aspect, the present application provides a kit, which may comprise the antigen-binding protein of the present application, the polypeptide of the present application, the nucleic acid of the present application, the vector of the present application, the cell of the present application, and/or the pharmaceutical composition of the present application.

In another aspect, the present application provides a method for stimulating an immune response, which may comprise administering the antigen-binding protein of the present application, the poly peptide of the present application, the nucleic acid of the present application, the vector of the present application, the cell of the present application, the pharmaceutical composition of the present application and/or the kit of the present application. For example, the method for stimulating an immune response may be in vitro and/or ex vivo. For example, the method for stimulating an immune response may be a method for a non-therapeutic purpose. In one aspect, stimulating the immune response may be increasing the capacity to kill target cells, decreasing the ability to release cytokines, and/or increasing the activation level of cells.

### Methods of Treatment

In another aspect, the present application provides a use of the antigen-binding protein of the present application, the polypeptide of the present application, the nucleic acid of the present application, the vector of the present application, the cell of the present application, the pharmaceutical composition of the present application and/or the kit of the present application in preparation of a medicament, said medicament is used for preventing, alleviating and/or treating a tumor. For example, the tumor may comprise solid tumor and/or hematologic tumor. For example, the tumor are selected from the group consisting of: gastric cancer, liver cancer, and leukemia. For example, the tumor of the present application may be selected from pan-cancer species, for example, the tumor may comprise a tumor that are positive to tumor antigens or have high expression of tumor antigens. For example, the tumor is selected from the group consisting of: lung cancer, breast cancer, liver cancer, brain cancer, cervical cancer, ovarian cancer, kidney cancer, testicular cancer, prostate cancer, and neuroblastoma.

In another aspect, the present application provides the antigen-binding protein of the present application, the polypeptide of the present application, the nucleic acid of the present application, the vector of the present application, the cell of the present application, the pharmaceutical composition of the present application and/or the kit of the present application, which can be used for preventing, alleviating and/or treating a tumor. For example, the tumor may comprise solid tumor and/or hematologic tumor. For example, the tumor are selected from the group consisting of: gastric cancer, liver cancer, and leukemia. For example, the tumor of the present application may be selected from pan-cancer species, for example, the tumor may comprise tumor that are positive to tumor antigens or have high expression of tumor antigens. For example, the tumor is selected from the group consisting of: lung cancer, breast cancer, liver cancer, brain cancer, cervical cancer, ovarian cancer, kidney cancer, testicular cancer, prostate cancer, and neuroblastoma.

In another aspect, the present application provides a method for preventing, alleviating and/or treating a tumor, comprising administering the antigen-binding protein of the present application, the polypeptide of the present application, the nucleic acid of the present application, the vector of the present application, the cell of the present application, the pharmaceutical composition of the present application and/or the kit of the present application. For example, the tumor may comprise solid tumor and/or hematologic tumor. For example, the tumor are selected from the group consisting of: gastric cancer, liver cancer, and leukemia. For example, the tumor of the present application may be selected from pan-cancer species, for example, the tumor may comprise a tumor that are positive to tumor antigens or have high expression of tumor antigens. For example, the tumor are selected from the group consisting of: lung cancer, breast cancer, liver cancer, brain cancer, cervical cancer, ovarian cancer, kidney cancer, testicular cancer, prostate cancer, and neuroblastoma.

In another aspect, the present disclosure provides methods for treating, preventing, reducing, ameliorating, inhibiting, delaying, or eliminating a disease or disorder in a subject in need thereof.

In some embodiments, the disease is a tumor.

In some embodiments, the tumor is a solid tumor, a hematologic malignancy, or both. Representative tumors include ovarian cancer, brain tumor, glioblastoma, mantle cell lymphoma (MCL), acute lymphoblastic leukemia (ALL), acute myeloid leukemia (AML), chronic lymphocytic leukemia (CLL), non-Hodgkin lymphoma, Hodgkin lymphoma, multiple myeloma, melanoma, lung cancer, breast cancer, colorectal cancer, pancreatic cancer, gastric cancer, liver cancer, prostate cancer, renal cancer, head and neck cancer, neuroblastoma, and mesothelioma.

The methods comprise administering to a subject a therapeutically effective amount of a cell expressing the chimeric antigen receptor disclosed herein. In some embodiments, the cell is an immune effector cell. In some embodiments, the immune effector cell comprises a T cell, a cytotoxic T cell, a helper T cell, a memory T cell, a stem cell memory T cell, a central memory T cell, an effector memory T cell, a natural killer (NK) cell, an NKT cell, a macrophage, a γδ T cell, or combinations thereof. The cells may be autologous, allogeneic, syngeneic, or xenogeneic.

In some embodiments, the methods comprise administration of a nucleic acid, vector, viral vector, or delivery vehicle encoding the chimeric antigen receptor.

In some embodiments, the chimeric antigen receptor is expressed ex vivo prior to administration.

In some embodiments, the chimeric antigen receptor is expressed in vivo following administration.

### Routes of Administration

The cells, nucleic acids, vectors, or pharmaceutical compositions described herein may be administered by any suitable route.

In some embodiments, administration is intravenous. In some embodiments, administration is intratumoral. In some embodiments, administration is intrathecal. In some embodiments, administration is intracerebral. In some embodiments, administration is intracerebroventricular. In some embodiments, administration is intraperitoneal. In some embodiments, administration is intralymphatic. In some embodiments, administration is systemic administration.

In certain embodiments involving brain tumors, administration is performed directly into the tumor, the tumor resection cavity, the cerebrospinal fluid, or the ventricular system.

### Dosage and Administration Regimens

The therapeutically effective amount may vary according to the disease, tumor burden, age, body weight, clinical condition, and treatment regimen of the subject.

In some embodiments, about 10⁴ to about 10¹⁰ cells are administered. In some embodiments, about 10⁵ to about 10⁹ cells are administered. In some embodiments, about 10⁶ to about 10⁸ cells are administered. In some embodiments, about 10⁴ to about 10⁸ cells/kg body weight are administered.

The cells may be administered as a single administration.

Alternatively, the cells may be administered in multiple administrations.

In some embodiments, the cells are administered two, three, four, five, or more times. The interval between administrations may range from about 1 day to about 12 months. In some embodiments, the interval is about 1 week to about 8 months. In some embodiments, the interval is about 2 weeks to about 6 months. In some embodiments, the interval is about 3 weeks to about 6 months. In some embodiments, the interval is about 1 week to about 3 months. In some embodiments, the interval is about 1 month to about 6 months. In some embodiments, the interval is about 1 month to about 2 months.

Administration may occur weekly, biweekly, monthly, quarterly, or according to disease progression.

In some embodiments, the subject receives a single dose of about 3.0×10⁵, 1×10⁶, 2×10⁶, 3×10⁶, 5×10⁶, 1×10⁷, or 2×10⁷ cells.

In some embodiments, the subject receives a single dose of about 10⁵ to about 10¹⁰ cells, about 10⁵ to about 10⁹ cells, about 10⁶ to about 10⁸ cells, about 5×10⁶ to about 5×10⁷ cells, about 3.0×10⁵ to about 2×10⁶ cells, about 10⁶ to about 3×10⁶ cells, about 10⁵ to about 10⁷ cells.

### Patient Population

The subject may be a human subject. In some embodiments, the subject is an adult. In some embodiments, the subject is a pediatric subject. The subject may be newly diagnosed, relapsed, refractory, recurrent, treatment-resistant, or metastatic. The methods may be used as first-line therapy, second-line therapy, salvage therapy, maintenance therapy, neoadjuvant therapy, adjuvant therapy, or consolidation therapy.

In another aspect, the present application further provides embodiments as follows:
1. A group of improved antigen-binding protein G1, wherein the G1 is capable of binding to CD24, and the enhanced effect of cells expressing said G1 on the killing ability of CD24-positive tumor cells, or the ability of cells expressing said G1 to express cytokines after being stimulated by CD24-positive tumor cells, is higher than that of cells expressing the existing CD24 antigen-binding proteins.
2. The antigen-binding protein G1 of Embodiment 1, wherein the cytokines comprise IFN-γ, and/or IL-2.
3. A group of further improved antigen-binding protein G2 based on the G1 of any one of Embodiments 1 to 2, wherein the G2 is capable of binding to CD24, G2-expressing cells maintain a capacity to kill the CD24-positive tumor cells, and an ability of expressing cytokines after stimulation by the CD24-positive tumor cells is at least about 50% lower than that of cells expressing G1.
4. A group of further improved antigen-binding protein G3 based on the G1 of any one of Embodiments 1 to 2 or based on the G2 of Embodiment 3 for use in expression in cells, wherein the G3-expressing cells co-express chemokine receptors, and preferably, the chemokine receptors comprise CXCR2.
5. A group of further improved antigen-binding protein G3 based on the G1 of any one of Embodiments 1 to 2 or based on the G2 of Embodiment 3 for use in expression in cells, wherein the G3-expressing cells co-express cytokines, and preferably, the cytokines comprise IL-12β.
6. The antigen-binding protein of any one of Embodiments 1 to 5, wherein the antigen-binding fragment comprises a light chain variable region and a heavy chain variable region, with a linker between the light chain variable region and the heavy chain variable region, the linker having a length of 10 amino acids or less.
7. The antigen-binding protein of Embodiment 6, wherein the linker has a length of 5 amino acids or less.
8. The antigen-binding protein of any one of Embodiments 1 to 7, wherein said antigen-binding protein comprises H-CDRs 1 to 3 in said heavy chain variable region as set forth in SEQ ID NO: 23 or 24, and L-CDRs 1 to 3 in said light chain variable region as set forth in SEQ ID NO: 25 or 26.
9. The antigen-binding protein of any one of Embodiments 1 to 8, wherein the antigen-binding protein comprises H-CDRs 1 to 3 in the heavy chain variable region as set forth in SEQ ID NOs: 42 to 44, and L-CDRs 1 to 3 in the light chain variable region as set forth in SEQ ID NOs: 45 to 47.
10. The antigen-binding protein of any one of Embodiments 1 to 9, wherein the signal peptide comprises a sequence as set forth in SEQ ID NO: 22.
11. The antigen-binding protein of any one of Embodiments 1 to 10, wherein the antigen-binding protein further comprises a hinge domain, which is directly or indirectly linked to said antigen-binding domain.
12. The antigen-binding protein of Embodiment 11, wherein the hinge domain comprises a hinge domain of CD8 or CD28, or of a functional fragment thereof.
13. The antigen-binding protein of any one of Embodiments 11 to 12, wherein the hinge domain comprises a sequence as set forth in SEQ ID NO: 32 or 33.
14. The antigen-binding protein of any one of Embodiments 1 to 13, wherein the antigen-binding protein further comprises a transmembrane domain, which is directly or indirectly linked to the hinge domain.
15. The antigen-binding protein of Embodiment 14, wherein the transmembrane domain comprises a transmembrane domain of CD8 or CD28, or of a functional fragment thereof.
16. The antigen-binding protein of any one of Embodiments 14 to 15, wherein the transmembrane domain comprises a sequence as set forth in SEQ ID NO: 34 or 35.
17. The antigen-binding protein of any one of Embodiments 1 to 16, wherein the antigen-binding protein further comprises an intracellular domain, which is directly or indirectly linked to the transmembrane domain.
18. The antigen-binding protein of Embodiment 17, wherein the intracellular domain comprises a costimulatory domain, which comprises a costimulatory domain of 41-BB, CD28, or OX40, or of a functional fragment thereof.
19. The antigen-binding protein of Embodiment 18, wherein the antigen-binding protein comprises one or more of the costimulatory domains.
20. The antigen-binding protein of any one of Embodiments 18 to 19, wherein the costimulatory domain comprises a sequence as set forth in SEQ ID NO: 36, 37, or 38.
21. The antigen-binding protein of any one of Embodiments 1 to 20, wherein the intracellular domain comprises a signaling domain, which comprises a signaling domain of CD3ζ or a functional segment thereof.
22. The antigen-binding protein of Embodiment 21, wherein the intracellular signaling domain comprises a sequence as set forth in SEQ ID NO: 39.
23. The antigen-binding protein of any one of Embodiments 1 to 22, wherein the antigen-binding protein comprises a chimeric antigen receptor.
24. The antigen-binding protein of Embodiments 1 to 23, wherein the antigen-binding protein is selected from a sequence as set forth in any one of SEQ ID NOs: 1 to 21, 48 and 49.
25. A polypeptide, comprising the antigen-binding protein of any one of Embodiments 1 to 24.
26. The polypeptide of Embodiment 25, wherein the polypeptide further comprises a tagged protein, which comprises a sequence as set forth in SEQ ID NO: 40.
27. The polypeptide of Embodiment 26, wherein the tagged protein and the antigen-binding protein of the polypeptide are linked via a sequence as set forth in SEQ ID NO: 41.
28. A nucleic acid, encoding the antigen-binding protein of any one of Embodiments 1 to 24 and/or the polypeptide of any one of Embodiments 25 to 27.
29. A vector, comprising the nucleic acid of Embodiment 28.
30. A cell, comprising the antigen-binding protein of any one of Embodiments 1 to 24, the polypeptide of any one of Embodiments 25 to 27, the nucleic acid of Embodiment 28, and/or the vector of Embodiment 29.
31. The cell of Embodiment 30, wherein the cell comprises an immune cell.
32. The cell of any one of Embodiments 30 to 31, wherein the cell comprises an NK cell and/or a T cell.
33. A preparation method for the antigen-binding protein of any one of Embodiments 1 to 24 and/or the polypeptide of any one of Embodiments 25 to 27, wherein the preparation method comprises culturing the cell of any one of Embodiments 30 to 32 under conditions allowing expression of the antigen-binding protein and/or the polypeptide.
34. A composition, comprising the antigen-binding protein of any one of Embodiments 1 to 24, the polypeptide of any one of Embodiments 25 to 27, the nucleic acid of Embodiment 28, the vector of Embodiment 29, and/or the cell of any one of Embodiments 30 to 32, and optionally, a pharmaceutically acceptable carrier.
35. A kit, comprising the antigen-binding protein of any one of Embodiments 1 to 24, the polypeptide of any one of Embodiments 25 to 27, the nucleic acid of Embodiment 28, the vector of Embodiment 29, the cell of any one of Embodiments 30 to 32, and/or the composition of Embodiment 34.
36. A method for stimulating an immune response, comprising administering the antigen-binding protein of any one of Embodiments 1 to 24, the polypeptide of any one of Embodiments 25 to 27, the nucleic acid of Embodiment 28, the vector of Embodiment 29, the cell of any one of Embodiments 30 to 32, the composition of Embodiment 34 and/or the kit of Embodiment 35.
37. The use of the antigen-binding protein of any one of Embodiments 1 to 24, the polypeptide of any one of Embodiments 25 to 27, the nucleic acid of Embodiment 28, the vector of Embodiment 29, the cell of any one of Embodiments 30 to 32, the composition of Embodiment 34 and/or the kit of Embodiment 35 in preparation of a medicament, said medicament is used for preventing, alleviating, and/or treating a tumor.
38. The use of Embodiment 37, wherein said tumor comprise solid tumor and/or hematologic tumor.
39. The use of any one of Embodiments 37 to 38, wherein the tumors are selected from the group consisting of: lung cancer, breast cancer, liver cancer, brain cancer, cervical cancer, ovarian cancer, kidney cancer, testicular cancer, prostate cancer, and neuroblastoma.

In another aspect, the present application further provides embodiments as follows:
1. A chimeric antigen receptor (CAR), comprising:
   (a) a CD24-binding domain comprising a heavy chain variable region (VH) and a light chain variable region (VL),
   wherein the VH comprises a HCDR1 comprising an amino acid sequence as set forth in SEQ ID NO: 42, a HCDR2 comprising an amino acid sequence as set forth in SEQ ID NO: 43, and a HCDR3 comprising an amino acid sequence as set forth in SEQ ID NO: 44, and
   wherein the VL comprises a LCDR1 comprising an amino acid sequence as set forth in SEQ ID NO: 45, a LCDR2 comprising an amino acid sequence as set forth in SEQ ID NO: 46, and a LCDR3 comprising an amino acid sequence as set forth in SEQ ID NO: 47,
   wherein the VH and the VL are linked by a linker comprising an amino acid sequence selected from the group consisting of SEQ ID NOs: 27, and 54-57;
   (b) a hinge region;
   (c) a transmembrane domain; and
   (d) an intracellular signaling domain.
2. The chimeric antigen receptor of Embodiment 1, wherein the VH comprise an amino acid sequence as set forth in SEQ ID NO: 23 and the VL comprise an amino acid sequence as set forth in SEQ ID NO: 25.
3. The chimeric antigen receptor of Embodiment 1, wherein the VH comprise an amino acid sequence as set forth in SEQ ID NO: 24 and the VL comprise an amino acid sequence as set forth in SEQ ID NO: 26.
4. The chimeric antigen receptor of any one of Embodiments 1-3, wherein the hinge region comprises a CD8 hinge region or a CD28 hinge region.
5. The chimeric antigen receptor of Embodiment 4, wherein the hinge region comprises an amino acid sequence as set forth in any one of SEQ ID NOs: 32-33.
6. The chimeric antigen receptor of any one of Embodiments 1-5, wherein the transmembrane domain comprises a CD8 transmembrane domain or a CD28 transmembrane domain.
7. The chimeric antigen receptor of Embodiment 6, wherein the transmembrane domain comprises an amino acid sequence as set forth in any one of SEQ ID NOs: 34-35.
8. The chimeric antigen receptor of any one of Embodiments 1-7, wherein the intracellular signaling domain comprising a CD3ζ signaling domain.
9. The chimeric antigen receptor of Embodiment 8, wherein the CD3ζ signaling domain comprises an amino acid sequence as set forth in SEQ ID NO:39.
10. The chimeric antigen receptor of any one of Embodiments 1-9, further comprising one or more co-stimulatory domains.
11. The chimeric antigen receptor of Embodiment 10, wherein the one or more co-stimulatory domains are independently selected from 4-1-BB co-stimulatory domain, CD28 co-stimulatory domain, OX-40 co-stimulatory domain or a combination thereof.
12. The chimeric antigen receptor of Embodiment 11, wherein the co-stimulatory domain comprises an amino acid sequence as set forth in any one of SEQ ID NOs: 36-38.
13. The chimeric antigen receptor of any one of Embodiments 1-12, further comprising a signal peptide in the N-terminus of the CD24-binding domain.
14. The chimeric antigen receptor of Embodiment 13, wherein the signal peptide comprises a CD8 signal peptide.
15. The chimeric antigen receptor of Embodiment 14, wherein the signal peptide comprises an amino acid sequence as set forth in SEQ ID NO: 22.
16. The chimeric antigen receptor of any one of Embodiments 1-15, further comprising a cytokine or a chemokine receptor co-expressed with the CAR.
17. The chimeric antigen receptor of Embodiment 16, wherein the cytokine or a chemokine receptor is co-expressed with the CAR via a self-cleaving 2A peptide sequence or an internal ribosome entry site (IRES).
18. The chimeric antigen receptor of Embodiment 17, wherein the self-cleaving 2A peptide comprises P2A or T2A.
19. The chimeric antigen receptor of Embodiment 17, wherein the self-cleaving 2A peptide comprises an amino acid sequence as set forth in SEQ ID NO: 51 or 52.
20. The chimeric antigen receptor of any one of Embodiments 16-19, further comprising a CXCR2 co-expressed with the CAR.
21. The chimeric antigen receptor of Embodiment 20, wherein the CXCR2 comprises an amino acid sequence as set forth in SEQ ID NO: 53.
22. The chimeric antigen receptor of any one of Embodiments 16-19, further comprising an IL-12β co-expressed with the CAR.
23. The chimeric antigen receptor of Embodiment 22, wherein the IL-12β comprises an amino acid sequence as set forth in SEQ ID NO: 50.
24. The chimeric antigen receptor of Embodiment 22, comprises an amino acid sequence as set forth in any one of SEQ ID NOs: 1-21 and 48-49.
25. A nucleic acid molecule encoding the chimeric antigen receptor (CAR) of any one of Embodiments 1-24.
26. A vector comprising the nucleic acid molecule of Embodiment 25.
27. The vector of Embodiment 26, wherein the vector is a viral vector, a plasmid, or an mRNA.
28. The vector of Embodiment 27, wherein the viral vector is selected from the group consisting of a lentiviral vector, a retroviral vector, an adenoviral vector, and an adeno-associated viral (AAV) vector.
29. A cell expressing the chimeric antigen receptor (CAR) of any one of Embodiments 1-24.
30. A cell comprising the nucleic acid molecule of Embodiment 25, or the vector of any one of Embodiments 26-28.
31. The cell of any one of Embodiments 29-30, wherein the cell is an immune cell.
32. The cell of Embodiment 31, wherein the immune cell is T cell, NK cell, NKT cell, macrophage, or a combination thereof.
33. A pharmaceutical composition comprising:
   (a) a chimeric antigen receptor (CAR) of any one of Embodiments 1-24, a nucleic acid molecule of Embodiment 25, a vector of any one of Embodiments 26-28, or a cell of any one of Embodiments 29-32; and
   (b) a pharmaceutically acceptable carrier or excipient.
34. A method for treating or alleviating a tumor, comprising administering to a subject in need thereof a therapeutically effective amount of a cell expressing a chimeric antigen receptor (CAR) of any one of Embodiments 1-24, a nucleic acid molecule of Embodiment 25, a vector of any one of Embodiments 26-28, a cell of any one of Embodiments 29-32, or a pharmaceutically composition of Embodiment 33.
35. The method of Embodiment 34, wherein the tumor is a CD24 comprises a CD24-positive malignancy.
36. The method of any one of Embodiments 34-35, wherein the tumor is a solid tumor or a hematopoietic malignancy.
37. The method of any one of Embodiments 34-36, wherein the tumor is selected from the group consisting of ovarian cancer, brain cancer (e.g., glioblastoma), mantle cell lymphoma (MCL), and acute lymphoblastic leukemia (ALL).
38. The method of any one of Embodiments 34-37, wherein the cell is administered intravenously, intratumorally, intrathecally, intracerebrally or systemically.
39. The method of any one of Embodiments 34-38, wherein the cell is administered as a single administration.
40. The method of any one of Embodiments 34-38, wherein the cell is administered in multiple administrations.
41. The method of any one of Embodiment 40, wherein the administrations are separated by an interval of about 1 day to about 12 months
42. The method of any one of Embodiments 40-41, wherein the administrations are separated by an interval of about 1 week to about 8 months.
43. The method of any one of Embodiments 40-41, wherein the administrations are separated by an interval of about 2 weeks to about 6 months.
44. The method of any one of Embodiments 40-42, wherein the administrations are separated by an interval of about 1 month to about 3 months.
45. The method of any one of Embodiments 34-44, wherein the subject receives a single dose of about 1×10⁵ to about 1×10¹⁰ cells.
46. The method of any one of Embodiments 34-45, wherein the subject receives a single dose of about 1×10⁶ to about 1×10⁸ cells.
47. The method of any one of Embodiments 34-46, wherein the subject receives a single dose of about 5×10⁶ to about 5×10⁷ cells.
48. The method of any one of Embodiments 34-44, wherein the subject receives a single dose of about 3.0×10⁵, 1×10⁶, 2×10⁶, 3×10⁶, 5×10⁶, 1×10⁷, or 2×10⁷ cells.
49. The method of any one of Embodiments 34-48, further comprising administering a lymphodepleting agent to the subject prior to administration of the cell.
50. The method of Embodiment 49, wherein the lymphodepleting agent comprises cyclophosphamide, fludarabine, bendamustine, or a combination thereof.
51. The method of any one of Embodiments 34-50, further comprising administering an additional anti-tumor therapy.
52. The method of Embodiment 51, wherein the additional anti-tumor therapy comprises chemotherapy, radiotherapy, immunotherapy, targeted therapy, surgery, or a combination thereof.
53. The method of Embodiment 52, wherein the additional anti-tumor therapy comprises an immune checkpoint inhibitor.
54. The method of Embodiment 53, wherein the immune checkpoint inhibitor targets PD-1, PD-L1, or CTLA-4.
55. The method of any one of Embodiments 34-54, wherein the subject has relapsed or refractory cancer.
56. The method of any one of Embodiments 37-55, wherein the subject has ovarian cancer, glioblastoma, mantle cell lymphoma, or acute lymphoblastic leukemia.

Not wishing to be bound by any theory, the following examples are merely to illustrate the proteins, preparation methods, uses and the like of the present application, and are not intended to limit the scope of the invention of the present application.

### EXAMPLES

### Material and Methods

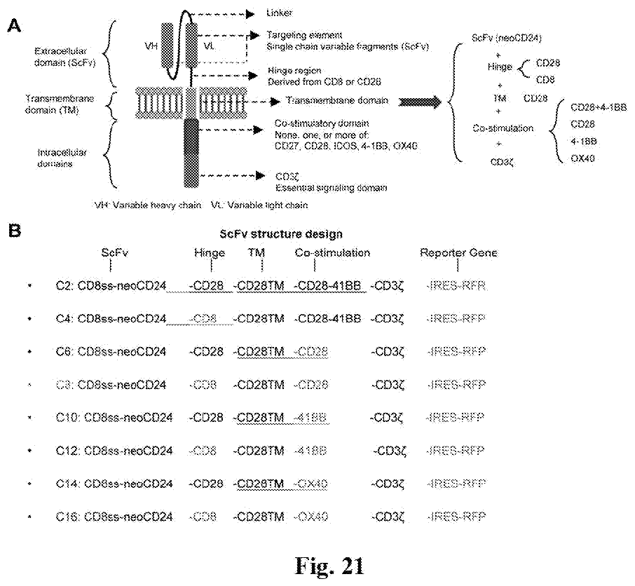

A CAR library targeting CD24 was constructed and screened for variants exhibiting different cytotoxicity and cytokine profiles upon stimulation with tumor cells. The library was generated by optimization of the single-chain variable fragment (ScFv) binding domain derived from neoCD24 monoclonal antibodies, which was linked to a hinge region, a transmembrane domain, and intracellular activation and co-stimulatory domains (FIG. 21A). Specifically, optimization was performed with respect to: (i) the ScFv region of the neoCD24 CAR constructs, (ii) hinge domains derived from either CD28 or CD8, and (iii) co-stimulatory domains including CD28, 4-1BB, CD28-4-1BB, or OX40 (FIG. 21B).

| SEQ ID NO | Remark | Sequence |
|---|---|---|
| 1 | CAR-C1 | EVQFVESGGGLVQPGGSLKLSCAASGVTFSEAWMDWVRQASGKG |
| | | LEWVAEIRDKPNSYVTYYAESVKGRFTISRDDSKSTAYLQMNSLKT |
| | | EDTAIYYCTGAMDYWGQGTLVTVSSGGGGSGGGGSGGGGSDIVM |
| | | TQTPLSLSVTIGQPASISCKSSQSLLYSNGKTYLNWLLQKPGQSPKR |
| | | LIYQVSKLDPGIPDRFSGSGSETDFTLKISRVEAEDVGIYYCMQGSSL |
| | | PWTFGGGTKVEIKRSVTVSSAAAIEVMYPPPYLDNEKSNGTIIHVKG |
| | | KHLCPSPLFPGPSKPFWVLVVVGGVLACYSLLVTVAFIIFWVRSKRS |
| | | RLLHSDYMNMTPRRPGPTRKHYQPYAPPRDFAAYRSKRGRKKLLY |
| | | IFKQPFMRPVQTTQEEDGCSCRFPEEEEGGCELRVKFSRSADAPAY |
| | | QQGQNQLYNELNLGRREEYDVLDKRRGRDPEMGGKPQRRKNPQE |
| | | GLYNELQKDKMAEAYSEIGMKGERRRGKGHDGLYQGLSTATKDT |
| | | YDALHMQALPPR |
| 2 | CAR-C2 | EVKFEESGGGLVQPGGSIKLSCAASGVTFSEAWMDWVRQSPEKGL |
| | | EWVAEIRDKPNSYVTYYAESVKGRFTISRDDSKSRVYLQMNNLRT |
| | | EDTGIYYCTGAMDYWGQGTSVTVSSGSTGGGGSGGGGSGGGGSDI |
| | | VMTQTPLSLSVTIGQPASISCKSSQSLLYSNGKTYLNWLQQRPGQSP |
| | | KRLIYQVSKLDPGIPDRFSGSGSETDFTLKISRVEAEDLGIYYCMQG |
| | | SSLPWTFGGGTKLEIKRSVTVSSAAAIEVMYPPPYLDNEKSNGTIIH |
| | | VKGKHLCPSPLFPGPSKPFWVLVVVGGVLACYSLLVTVAFIIFWVR |
| | | SKRSRLLHSDYMNMTPRRPGPTRKHYQPYAPPRDFAAYRSKRGRK |
| | | KLLYIFKQPFMRPVQTTQEEDGCSCRFPEEEEGGCELRVKFSRSADA |
| | | PAYQQGQNQLYNELNLGRREEYDVLDKRRGRDPEMGGKPQRRKN |
| | | PQEGLYNELQKDKMAEAYSEIGMKGERRRGKGHDGLYQGLSTAT |
| | | KDTYDALHMQALPPR |
| 3 | CAR-C3 | EVQFVESGGGLVQPGGSLKLSCAASGVTFSEAWMDWVRQASGKG |
| | | LEWVAEIRDKPNSYVTYYAESVKGRFTISRDDSKSTAYLQMNSLKT |
| | | EDTAIYYCTGAMDYWGQGTLVTVSSGGGGSGGGGSGGGGSDIVM |
| | | TQTPLSLSVTIGQPASISCKSSQSLLYSNGKTYLNWLLQKPGQSPKR |
| | | LIYQVSKLDPGIPDRFSGSGSETDFTLKISRVEAEDVGIYYCMQGSSL |
| | | PWTFGGGTKVEIKASSHFVPVFLPAKPTTTPAPRPPTPAPTIASQPLS |
| | | LRPEACRPAAGGAVHTRGLDFACDFWVLVVVGGVLACYSLLVTV |
| | | AFIIFWVRSKRSRLLHSDYMNMTPRRPGPTRKHYQPYAPPRDFAAY |
| | | RSKRGRKKLLYIFKQPFMRPVQTTQEEDGCSCRFPEEEEGGCELRV |
| | | KFSRSADAPAYQQGQNQLYNELNLGRREEYDVLDKRRGRDPEMG |
| | | GKPQRRKNPQEGLYNELQKDKMAEAYSEIGMKGERRRGKGHDGL |
| | | YQGLSTATKDTYDALHMQALPPR |
| 4 | CAR-C4 | EVKFEESGGGLVQPGGSIKLSCAASGVTFSEAWMDWVRQSPEKGL |
| | | EWVAEIRDKPNSYVTYYAESVKGRFTISRDDSKSRVYLQMNNLRT |
| | | EDTGIYYCTGAMDYWGQGTSVTVSSGSTGGGGSGGGGSGGGGSDI |
| | | VMTQTPLSLSVTIGQPASISCKSSQSLLYSNGKTYLNWLQQRPGQSP |
| | | KRLIYQVSKLDPGIPDRFSGSGSETDFTLKISRVEAEDLGIYYCMQG |
| | | SSLPWTFGGGTKLEIKASSHFVPVFLPAKPTTTPAPRPPTPAPTIASQ |
| | | PLSLRPEACRPAAGGAVHTRGLDFACDFWVLVVVGGVLACYSLLV |
| | | TVAFIIFWVRSKRSRLLHSDYMNMTPRRPGPTRKHYQPYAPPRDFA |
| | | AYRSKRGRKKLLYIFKQPFMRPVQTTQEEDGCSCRFPEEEEGGCEL |
| | | RVKFSRSADAPAYQQGQNQLYNELNLGRREEYDVLDKRRGRDPE |
| | | MGGKPQRRKNPQEGLYNELQKDKMAEAYSEIGMKGERRRGKGHD |
| | | GLYQGLSTATKDTYDALHMQALPPR |
| 5 | CAR-C5 | EVQFVESGGGLVQPGGSLKLSCAASGVTFSEAWMDWVRQASGKG |
| | | LEWVAEIRDKPNSYVTYYAESVKGRFTISRDDSKSTAYLQMNSLKT |
| | | EDTAIYYCTGAMDYWGQGTLVTVSSGGGGSGGGGSGGGGSDIVM |
| | | TQTPLSLSVTIGQPASISCKSSQSLLYSNGKTYLNWLLQKPGQSPKR |
| | | LIYQVSKLDPGIPDRFSGSGSETDFTLKISRVEAEDVGIYYCMQGSSL |
| | | PWTFGGGTKVEIKRSVTVSSAAAIEVMYPPPYLDNEKSNGTIIHVKG |
| | | KHLCPSPLFPGPSKPFWVLVVVGGVLACYSLLVTVAFIIFWVRSKRS |
| | | RLLHSDYMNMTPRRPGPTRKHYQPYAPPRDFAAYRSRVKFSRSAD |
| | | APAYQQGQNQLYNELNLGRREEYDVLDKRRGRDPEMGGKPQRRK |
| | | NPQEGLYNELQKDKMAEAYSEIGMKGERRRGKGHDGLYQGLSTA |
| | | TKDTYDALHMQALPPR |
| 6 | CAR-C6 | EVKFEESGGGLVQPGGSIKLSCAASGVTFSEAWMDWVRQSPEKGL |
| | | EWVAEIRDKPNSYVTYYAESVKGRFTISRDDSKSRVYLQMNNLRT |
| | | EDTGIYYCTGAMDYWGQGTSVTVSSGSTGGGGSGGGGSGGGGSDI |
| | | VMTQTPLSLSVTIGQPASISCKSSQSLLYSNGKTYLNWLQQRPGQSP |
| | | KRLIYQVSKLDPGIPDRFSGSGSETDFTLKISRVEAEDLGIYYCMQG |
| | | SSLPWTFGGGTKLEIKRSVTVSSAAAIEVMYPPPYLDNEKSNGTIIH |
| | | VKGKHLCPSPLFPGPSKPFWVLVVVGGVLACYSLLVTVAFIIFWVR |
| | | SKRSRLLHSDYMNMTPRRPGPTRKHYQPYAPPRDFAAYRSRVKFS |
| | | RSADAPAYQQGQNQLYNELNLGRREEYDVLDKRRGRDPEMGGKP |
| | | QRRKNPQEGLYNELQKDKMAEAYSEIGMKGERRRGKGHDGLYQG |
| | | LSTATKDTYDALHMQALPPR |
| 7 | CAR-C7 | EVQFVESGGGLVQPGGSLKLSCAASGVTFSEAWMDWVRQASGKG |
| | | LEWVAEIRDKPNSYVTYYAESVKGRFTISRDDSKSTAYLQMNSLKT |
| | | EDTAIYYCTGAMDYWGQGTLVTVSSGGGGSGGGGSGGGGSDIVM |
| | | TQTPLSLSVTIGQPASISCKSSQSLLYSNGKTYLNWLLQKPGQSPKR |
| | | LIYQVSKLDPGIPDRFSGSGSETDFTLKISRVEAEDVGIYYCMQGSSL |
| | | PWTFGGGTKVEIKASSHFVPVFLPAKPTTTPAPRPPTPAPTIASQPLS |
| | | LRPEACRPAAGGAVHTRGLDFACDFWVLVVVGGVLACYSLLVTV |
| | | AFIIFWVRSKRSRLLHSDYMNMTPRRPGPTRKHYQPYAPPRDFAAY |
| | | RSRVKFSRSADAPAYQQGQNQLYNELNLGRREEYDVLDKRRGRDP |
| | | EMGGKPQRRKNPQEGLYNELQKDKMAEAYSEIGMKGERRRGKGH |
| | | DGLYQGLSTATKDTYDALHMQALPPR |
| 8 | CAR-C8 | EVKFEESGGGLVQPGGSIKLSCAASGVTFSEAWMDWVRQSPEKGL |
| | | EWVAEIRDKPNSYVTYYAESVKGRFTISRDDSKSRVYLQMNNLRT |
| | | EDTGIYYCTGAMDYWGQGTSVTVSSGSTGGGGSGGGGSGGGGSDI |
| | | VMTQTPLSLSVTIGQPASISCKSSQSLLYSNGKTYLNWLQQRPGQSP |
| | | KRLIYQVSKLDPGIPDRFSGSGSETDFTLKISRVEAEDLGIYYCMQG |
| | | SSLPWTFGGGTKLEIKASSHFVPVFLPAKPTTTPAPRPPTPAPTIASQ |
| | | PLSLRPEACRPAAGGAVHTRGLDFACDFWVLVVVGGVLACYSLLV |
| | | TVAFIIFWVRSKRSRLLHSDYMNMTPRRPGPTRKHYQPYAPPRDFA |
| | | AYRSRVKFSRSADAPAYQQGQNQLYNELNLGRREEYDVLDKRRG |
| | | RDPEMGGKPQRRKNPQEGLYNELQKDKMAEAYSEIGMKGERRRG |
| | | KGHDGLYQGLSTATKDTYDALHMQALPPR |
| 9 | CAR-C9 | EVQFVESGGGLVQPGGSLKLSCAASGVTFSEAWMDWVRQASGKG |
| | | LEWVAEIRDKPNSYVTYYAESVKGRFTISRDDSKSTAYLQMNSLKT |
| | | EDTAIYYCTGAMDYWGQGTLVTVSSGGGGSGGGGSGGGGSDIVM |
| | | TQTPLSLSVTIGQPASISCKSSQSLLYSNGKTYLNWLLQKPGQSPKR |
| | | LIYQVSKLDPGIPDRFSGSGSETDFTLKISRVEAEDVGIYYCMQGSSL |
| | | PWTFGGGTKVEIKRSVTVSSAAAIEVMYPPPYLDNEKSNGTIIHVKG |
| | | KHLCPSPLFPGPSKPFWVLVVVGGVLACYSLLVTVAFIIFWVKRGR |
| | | KKLLYIFKQPFMRPVQTTQEEDGCSCRFPEEEEGGCELRVKFSRSAD |
| | | APAYQQGQNQLYNELNLGRREEYDVLDKRRGRDPEMGGKPQRRK |
| | | NPQEGLYNELQKDKMAEAYSEIGMKGERRRGKGHDGLYQGLSTA |
| | | TKDTYDALHMQALPPR |
| 10 | CAR-C10 | EVKFEESGGGLVQPGGSIKLSCAASGVTFSEAWMDWVRQSPEKGL |
| | | EWVAEIRDKPNSYVTYYAESVKGRFTISRDDSKSRVYLQMNNLRT |
| | | EDTGIYYCTGAMDYWGQGTSVTVSSGSTGGGGSGGGGSGGGGSDI |
| | | VMTQTPLSLSVTIGQPASISCKSSQSLLYSNGKTYLNWLQQRPGQSP |
| | | KRLIYQVSKLDPGIPDRFSGSGSETDFTLKISRVEAEDLGIYYCMQG |
| | | SSLPWTFGGGTKLEIKRSVTVSSAAAIEVMYPPPYLDNEKSNGTIIH |
| | | VKGKHLCPSPLFPGPSKPFWVLVVVGGVLACYSLLVTVAFIIFWVK |
| | | RGRKKLLYIFKQPFMRPVQTTQEEDGCSCRFPEEEEGGCELRVKFS |
| | | RSADAPAYQQGQNQLYNELNLGRREEYDVLDKRRGRDPEMGGKP |
| | | QRRKNPQEGLYNELQKDKMAEAYSEIGMKGERRRGKGHDGLYQG |
| | | LSTATKDTYDALHMQALPPR |
| 11 | CAR-C11 | EVQFVESGGGLVQPGGSLKLSCAASGVTFSEAWMDWVRQASGKG |
| | | LEWVAEIRDKPNSYVTYYAESVKGRFTISRDDSKSTAYLQMNSLKT |
| | | EDTAIYYCTGAMDYWGQGTLVTVSSGGGGSGGGGSGGGGSDIVM |
| | | TQTPLSLSVTIGQPASISCKSSQSLLYSNGKTYLNWLLQKPGQSPKR |
| | | LIYQVSKLDPGIPDRFSGSGSETDFTLKISRVEAEDVGIYYCMQGSSL |
| | | PWTFGGGTKVEIKASSHFVPVFLPAKPTTTPAPRPPTPAPTIASQPLS |
| | | LRPEACRPAAGGAVHTRGLDFACDFWVLVVVGGVLACYSLLVTV |
| | | AFIIFWVKRGRKKLLYIFKQPFMRPVQTTQEEDGCSCRFPEEEEGGC |
| | | ELRVKFSRSADAPAYQQGQNQLYNELNLGRREEYDVLDKRRGRDP |
| | | EMGGKPQRRKNPQEGLYNELQKDKMAEAYSEIGMKGERRRGKGH |
| | | DGLYQGLSTATKDTYDALHMQALPPR |
| 12 | CAR-C12 | EVKFEESGGGLVQPGGSIKLSCAASGVTFSEAWMDWVRQSPEKGL |
| | | EWVAEIRDKPNSYVTYYAESVKGRFTISRDDSKSRVYLQMNNLRT |
| | | EDTGIYYCTGAMDYWGQGTSVTVSSGSTGGGGSGGGGSGGGGSDI |
| | | VMTQTPLSLSVTIGQPASISCKSSQSLLYSNGKTYLNWLQQRPGQSP |
| | | KRLIYQVSKLDPGIPDRFSGSGSETDFTLKISRVEAEDLGIYYCMQG |
| | | SSLPWTFGGGTKLEIKASSHFVPVFLPAKPTTTPAPRPPTPAPTIASQ |
| | | PLSLRPEACRPAAGGAVHTRGLDFACDFWVLVVVGGVLACYSLLV |
| | | TVAFIIFWVKRGRKKLLYIFKQPFMRPVQTTQEEDGCSCRFPEEEEG |
| | | GCELRVKFSRSADAPAYQQGQNQLYNELNLGRREEYDVLDKRRG |
| | | RDPEMGGKPQRRKNPQEGLYNELQKDKMAEAYSEIGMKGERRRG |
| | | KGHDGLYQGLSTATKDTYDALHMQALPPR |
| 13 | CAR-C13 | EVQFVESGGGLVQPGGSLKLSCAASGVTFSEAWMDWVRQASGKG |
| | | LEWVAEIRDKPNSYVTYYAESVKGRFTISRDDSKSTAYLQMNSLKT |
| | | EDTAIYYCTGAMDYWGQGTLVTVSSGGGGSGGGGSGGGGSDIVM |
| | | TQTPLSLSVTIGQPASISCKSSQSLLYSNGKTYLNWLLQKPGQSPKR |
| | | LIYQVSKLDPGIPDRFSGSGSETDFTLKISRVEAEDVGIYYCMQGSSL |
| | | PWTFGGGTKVEIKRSVTVSSAAAIEVMYPPPYLDNEKSNGTIIHVKG |
| | | KHLCPSPLFPGPSKPFWVLVVVGGVLACYSLLVTVAFIIFWVRRDQ |
| | | RLPPDAHKPPGGGSFRTPIQEEQADAHSTLAKIRVKFSRSADAPAYQ |
| | | QGQNQLYNELNLGRREEYDVLDKRRGRDPEMGGKPQRRKNPQEG |
| | | LYNELQKDKMAEAYSEIGMKGERRRGKGHDGLYQGLSTATKDTY |
| | | DALHMQALPPR |
| 14 | CAR-C14 | EVKFEESGGGLVQPGGSIKLSCAASGVTFSEAWMDWVRQSPEKGL |
| | | EWVAEIRDKPNSYVTYYAESVKGRFTISRDDSKSRVYLQMNNLRT |
| | | EDTGIYYCTGAMDYWGQGTSVTVSSGSTGGGGSGGGGSGGGGSDI |
| | | VMTQTPLSLSVTIGQPASISCKSSQSLLYSNGKTYLNWLQQRPGQSP |
| | | KRLIYQVSKLDPGIPDRFSGSGSETDFTLKISRVEAEDLGIYYCMQG |
| | | SSLPWTFGGGTKLEIKRSVTVSSAAAIEVMYPPPYLDNEKSNGTIIH |
| | | VKGKHLCPSPLFPGPSKPFWVLVVVGGVLACYSLLVTVAFIIFWVR |
| | | RDQRLPPDAHKPPGGGSFRTPIQEEQADAHSTLAKIRVKFSRSADAP |
| | | AYQQGQNQLYNELNLGRREEYDVLDKRRGRDPEMGGKPQRRKNP |
| | | QEGLYNELQKDKMAEAYSEIGMKGERRRGKGHDGLYQGLSTATK |
| | | DTYDALHMQALPPR |
| 15 | CAR-C15 | EVQFVESGGGLVQPGGSLKLSCAASGVTFSEAWMDWVRQASGKG |
| | | LEWVAEIRDKPNSYVTYYAESVKGRFTISRDDSKSTAYLQMNSLKT |
| | | EDTAIYYCTGAMDYWGQGTLVTVSSGGGGSGGGGSGGGGSDIVM |
| | | TQTPLSLSVTIGQPASISCKSSQSLLYSNGKTYLNWLLQKPGQSPKR |
| | | LIYQVSKLDPGIPDRFSGSGSETDFTLKISRVEAEDVGIYYCMQGSSL |
| | | PWTFGGGTKVEIKASSHFVPVFLPAKPTTTPAPRPPTPAPTIASQPLS |
| | | LRPEACRPAAGGAVHTRGLDFACDFWVLVVVGGVLACYSLLVTV |
| | | AFIIFWVRRDQRLPPDAHKPPGGGSFRTPIQEEQADAHSTLAKIRVK |
| | | FSRSADAPAYQQGQNQLYNELNLGRREEYDVLDKRRGRDPEMGG |
| | | KPQRRKNPQEGLYNELQKDKMAEAYSEIGMKGERRRGKGHDGLY |
| | | QGLSTATKDTYDALHMQALPPR |
| 16 | CAR-C16 | EVKFEESGGGLVQPGGSIKLSCAASGVTFSEAWMDWVRQSPEKGL |
| | | EWVAEIRDKPNSYVTYYAESVKGRFTISRDDSKSRVYLQMNNLRT |
| | | EDTGIYYCTGAMDYWGQGTSVTVSSGSTGGGGSGGGGSGGGGSDI |
| | | VMTQTPLSLSVTIGQPASISCKSSQSLLYSNGKTYLNWLQQRPGQSP |
| | | KRLIYQVSKLDPGIPDRFSGSGSETDFTLKISRVEAEDLGIYYCMQG |
| | | SSLPWTFGGGTKLEIKASSHFVPVFLPAKPTTTPAPRPPTPAPTIASQ |
| | | PLSLRPEACRPAAGGAVHTRGLDFACDFWVLVVVGGVLACYSLLV |
| | | TVAFIIFWVRRDQRLPPDAHKPPGGGSFRTPIQEEQADAHSTLAKIR |
| | | VKFSRSADAPAYQQGQNQLYNELNLGRREEYDVLDKRRGRDPEM |
| | | GGKPQRRKNPQEGLYNELQKDKMAEAYSEIGMKGERRRGKGHDG |
| | | LYQGLSTATKDTYDALHMQALPPR |
| 17 | CAR-C23 | EVQFVESGGGLVQPGGSLKLSCAASGVTFSEAWMDWVRQASGKG |
| | | LEWVAEIRDKPNSYVTYYAESVKGRFTISRDDSKSTAYLQMNSLKT |
| | | EDTAIYYCTGAMDYWGQGTLVTVSSGGGGSGGGGSGGGGSDIVM |
| | | TQTPLSLSVTIGQPASISCKSSQSLLYSNGKTYLNWLLQKPGQSPKR |
| | | LIYQVSKLDPGIPDRFSGSGSETDFTLKISRVEAEDVGIYYCMQGSSL |
| | | PWTFGGGTKVEIKASSHFVPVFLPAKPTTTPAPRPPTPAPTIASQPLS |
| | | LRPEACRPAAGGAVHTRGLDFACDIYIWAPLAGTCGVLLLSLVITL |
| | | YCRSKRSRLLHSDYMNMTPRRPGPTRKHYQPYAPPRDFAAYRSRV |
| | | KFSRSADAPAYQQGQNQLYNELNLGRREEYDVLDKRRGRDPEMG |
| | | GKPQRRKNPQEGLYNELQKDKMAEAYSEIGMKGERRRGKGHDGL |
| | | YQGLSTATKDTYDALHMQALPPR |
| 18 | CAR-C27 | EVKFEESGGGLVQPGGSIKLSCAASGVTFSEAWMDWVRQSPEKGL |
| | | EWVAEIRDKPNSYVTYYAESVKGRFTISRDDSKSRVYLQMNNLRT |
| | | EDTGIYYCTGAMDYWGQGTSVTVSSGGGGSDIVMTQTPLSLSVTIG |
| | | QPASISCKSSQSLLYSNGKTYLNWLQQRPGQSPKRLIYQVSKLDPGI |
| | | PDRFSGSGSETDFTLKISRVEAEDLGIYYCMQGSSLPWTFGGGTKLE |
| | | IKRSVTVSSAAAIEVMYPPPYLDNEKSNGTIIHVKGKHLCPSPLFPGP |
| | | SKPFWVLVVVGGVLACYSLLVTVAFIIFWVRSKRSRLLHSDYMNM |
| | | TPRRPGPTRKHYQPYAPPRDFAAYRSRVKFSRSADAPAYQQGQNQ |
| | | LYNELNLGRREEYDVLDKRRGRDPEMGGKPQRRKNPQEGLYNEL |
| | | QKDKMAEAYSEIGMKGERRRGKGHDGLYQGLSTATKDTYDALH |
| | | MQALPPR |
| 19 | CAR-C28 | EVKFEESGGGLVQPGGSIKLSCAASGVTFSEAWMDWVRQSPEKGL |
| | | EWVAEIRDKPNSYVTYYAESVKGRFTISRDDSKSRVYLQMNNLRT |
| | | EDTGIYYCTGAMDYWGQGTSVTVSSGGGGSDIVMTQTPLSLSVTIG |
| | | QPASISCKSSQSLLYSNGKTYLNWLQQRPGQSPKRLIYQVSKLDPGI |
| | | PDRFSGSGSETDFTLKISRVEAEDLGIYYCMQGSSLPWTFGGGTKLE |
| | | IKASSHFVPVFLPAKPTTTPAPRPPTPAPTIASQPLSLRPEACRPAAG |
| | | GAVHTRGLDFACDFWVLVVVGGVLACYSLLVTVAFIIFWVRSKRS |
| | | RLLHSDYMNMTPRRPGPTRKHYQPYAPPRDFAAYRSRVKFSRSAD |
| | | APAYQQGQNQLYNELNLGRREEYDVLDKRRGRDPEMGGKPQRRK |
| | | NPQEGLYNELQKDKMAEAYSEIGMKGERRRGKGHDGLYQGLSTA |
| | | TKDTYDALHMQALPPR |
| 20 | CAR-C29 | EVQFVESGGGLVQPGGSLKLSCAASGVTFSEAWMDWVRQASGKG |
| | | LEWVAEIRDKPNSYVTYYAESVKGRFTISRDDSKSTAYLQMNSLKT |
| | | EDTAIYYCTGAMDYWGQGTLVTVSSGGGGGGGGSGGGGSGGGG |
| | | SDIVMTQTPLSLSVTIGQPASISCKSSQSLLYSNGKTYLNWLLQKPG |
| | | QSPKRLIYQVSKLDPGIPDRFSGSGSETDFTLKISRVEAEDVGIYYCM |
| | | QGSSLPWTFGGGTKVEIKASSHFVPVFLPAKPTTTPAPRPPTPAPTIA |
| | | SQPLSLRPEACRPAAGGAVHTRGLDFACDFWVLVVVGGVLACYSL |
| | | LVTVAFIIFWVRSKRSRLLHSDYMNMTPRRPGPTRKHYQPYAPPRD |
| | | FAAYRSRVKFSRSADAPAYQQGQNQLYNELNLGRREEYDVLDKRR |
| | | GRDPEMGGKPQRRKNPQEGLYNELQKDKMAEAYSEIGMKGERRR |
| | | GKGHDGLYQGLSTATKDTYDALHMQALPPR |
| 21 | CAR-C30 | EVKFEESGGGLVQPGGSIKLSCAASGVTFSEAWMDWVRQSPEKGL |
| | | EWVAEIRDKPNSYVTYYAESVKGRFTISRDDSKSRVYLQMNNLRT |
| | | EDTGIYYCTGAMDYWGQGTSVTVSSGGGGSGGGGSDIVMTQTPLS |
| | | LSVTIGQPASISCKSSQSLLYSNGKTYLNWLQQRPGQSPKRLIYQVS |
| | | KLDPGIPDRFSGSGSETDFTLKISRVEAEDLGIYYCMQGSSLPWTFG |
| | | GGTKLEIKASSHFVPVFLPAKPTTTPAPRPPTPAPTIASQPLSLRPEAC |
| | | RPAAGGAVHTRGLDFACDFWVLVVVGGVLACYSLLVTVAFIIFWV |
| | | RSKRSRLLHSDYMNMTPRRPGPTRKHYQPYAPPRDFAAYRSRVKF |
| | | SRSADAPAYQQGQNQLYNELNLGRREEYDVLDKRRGRDPEMGGK |
| | | PQRRKNPQEGLYNELQKDKMAEAYSEIGMKGERRRGKGHDGLYQ |
| | | GLSTATKDTYDALHMQALPPR |
| 22 | CD8ss | MALPVTALLLPLALLLHAARP |
| 23 | H3L3 VH | EVQFVESGGGLVQPGGSLKLSCAASGVTFSEAWMDWVRQASGKG |
| | | LEWVAEIRDKPNSYVTYYAESVKGRFTISRDDSKSTAYLQMNSLKT |
| | | EDTAIYYCTGAMDYWGQGTLVTVSS |
| 24 | PP6373 VH | EVKFEESGGGLVQPGGSIKLSCAASGVTFSEAWMDWVRQSPEKGL |
| | | EWVAEIRDKPNSYVTYYAESVKGRFTISRDDSKSRVYLQMNNLRT |
| | | EDTGIYYCTGAMDYWGQGTSVTVSS |
| 25 | H3L3 VL | DIVMTQTPLSLSVTIGQPASISCKSSQSLLYSNGKTYLNWLLQKPGQ |
| | | SPKRLIYQVSKLDPGIPDRFSGSGSETDFTLKISRVEAEDVGIYYCMQ |
| | | GSSLPWTFGGGTKVEIK |
| 26 | PP6373 VL | DIVMTQTPLSLSVTIGQPASISCKSSQSLLYSNGKTYLNWLQQRPGQ |
| | | SPKRLIYQVSKLDPGIPDRFSGSGSETDFTLKISRVEAEDLGIYYCMQ |
| | | GSSLPWTFGGGTKLEIK |
| 27 | linker1 | (GGGGS)n, n is an integer selected from 1 to 5 |
| 28 | linker2 | (G)n, n is an integer selected from 1 to 10 |
| 29 | linker3 | (GS)n, n is an integer selected from 1 to 5 |
| 30 | linker4 | (GGGS)n, n is an integer selected from 1 to 5 |
| 31 | linker5 | (GSGGS)n, n is 1 or 2 |
| 32 | CD28 hinge | IEVMYPPPYLDNEKSNGTIIHVKGKHLCPSPLFPGPSKP |
| 33 | CD8 hinge | SHFVPVFLPAKPTTTPAPRPPTPAPTIASQPLSLRPEACRPAAGGAVH |
| | | TRGLDFACD |
| 34 | CD28 TM | FWVLVVVGGVLACYSLLVTVAFIIFWV |
| | domain | |
| 35 | CD8 TM | IYIWAPLAGTCGVLLLSLVITLYC |
| | domain | |
| 36 | 4-1-BB co- | KRGRKKLLYIFKQPFMRPVQTTQEEDGCSCRFPEEEEGGCEL |
| | stimulation | |
| 37 | CD28 co- | RSKRSRLLHSDYMNMTPRRPGPTRKHYQPYAPPRDFAAYRS |
| | stimulation | |
| 38 | OX40 co- | RRDQRLPPDAHKPPGGGSFRTPIQEEQADAHSTLAKI |
| | stimulation | |
| 39 | CD3ζ | RVKFSRSADAPAYQQGQNQLYNELNLGRREEYDVLDKRRGRDPE |
| | | MGGKPQRRKNPQEGLYNELQKDKMAEAYSEIGMKGERRRGKGHD |
| | | GLYQGLSTATKDTYDALHMQALPPR |
| 40 | RFP | MASSEDVIKEFMRFKVRMEGSVNGHEFEIEGEGEGRPYEGTQTAKL |
| | | KVTKGGPLPFAWDILSPQFQYGSKAYVKHPADIPDYLKLSFPEGFK |
| | | WERVMNFEDGGVVTVTQDSSLQDGEFIYKVKLRGTNFPSDGPVMQ |
| | | KKTMGWEASTERMYPEDGALKGEIKMRLKLKDGGHYDAEVKTTY |
| | | MAKKPVQLPGAYKTDIKLDITSHNEDYTIVEQYERAEGRHSTGA |
| 41 | IRES DNA | CCACCTACATGGGGATCCCTCCCCCCCCCCTAACGTTACTGGCC |
| | | GAAGCCGCTTGGAATAAGGCCGGTGTGCGTTTGTCTATATGTTA |
| | | TTTTCCACCATATTGCCGTCTTTTGGCAATGTGAGGGCCCGGAAA |
| | | CCTGGCCCTGTCTTCTTGACGAGCATTCCTAGGGGTCTTTCCCCT |
| | | CTCGCCAAAGGAATGCAAGGTCTGTTGAATGTCGTGAAGGAAGC |
| | | AGTTCCTCTGGAAGCTTCTTGAAGACAAACAACGTCTGTAGCGA |
| | | CCCTTTGCAGGCAGCGGAACCCCCCACCTGGCGACAGGTGCCTC |
| | | TGCGGCCAAAAGCCACGTGTATAAGATACACCTGCAAAGGCGG |
| | | CACAACCCCAGTGCCACGTTGTGAGTTGGATAGTTGTGGAAAGA |
| | | GTCAAATGGCTCTCCTCAAGCGTATTCAACAAGGGGCTGAAGGA |
| | | TGCCCAGAAGGTACCCCATTGTATGGGATCTGATCTGGGGCCTC |
| | | GGTGCACATGCTTTACATGTGTTTAGTCGAGGTTAAAAAAACGT |
| | | CTAGGCCCCCCGAACCACGGGGACGTGGTTTTCCTTTGAAAAAC |
| | | ACGATAATACCATGACC |
| 42 | H3L3/PP6373 | GVTFSEAWMD |
| | VH CDR1 | |
| 43 | H3L3/PP6373 | EIRDKPNSYVTYYAESVKG |
| | VH CDR2 | |
| 44 | H3L3/PP6373 | AMDY |
| | VH CDR3 | |
| 45 | H3L3/PP6373 | KSSQSLLYSNGKTYLN |
| | VL CDR1 | |
| 46 | H3L3/PP6373 | QVSKLDP |
| | VL CDR2 | |
| 47 | H3L3/PP6373 | MQGSSLPWT |
| | VL CDR3 | |
| 48 | CAR-C31 | EVKFEESGGGLVQPGGSIKLSCAASGVTFSEAWMDWVRQSPEKGL |
| | (also referred | EWVAEIRDKPNSYVTYYAESVKGRFTISRDDSKSRVYLQMNNLRT |
| | to as CAR-C50, | EDTGIYYCTGAMDYWGQGTSVTVSSGGGGSDIVMTQTPLSLSVTIG |
| | C50 CAR or | QPASISCKSSQSLLYSNGKTYLNWLQQRPGQSPKRLIYQVSKLDPGI |
| | C50 herein) | PDRFSGSGSETDFTLKISRVEAEDLGIYYCMQGSSLPWTFGGGTKLE |
| | | IKASSHFVPVFLPAKPTTTPAPRPPTPAPTIASQPLSLRPEACRPAAG |
| | | GAVHTRGLDFACDFWVLVVVGGVLACYSLLVTVAFIIFWVRSKRS |
| | | RLLHSDYMNMTPRRPGPTRKHYQPYAPPRDFAAYRSRVKFSRSAD |
| | | APAYQQGQNQLYNELNLGRREEYDVLDKRRGRDPEMGGKPQRRK |
| | | NPQEGLYNELQKDKMAEAYSEIGMKGERRRGKGHDGLYQGLSTA |
| | | TKDTYDALHMQALPPRGSGATNFSLLKQAGDVEENPGPMEDFNM |
| | | ESDSFEDFWKGEDLSNYSYSSTLPPFLLDAAPCEPESLEINKYFVVII |
| | | YALVFLLSLLGNSLVMLVILYSRVGRSVTDVYLLNLALADLLFALT |
| | | LPIWAASKVNGWIFGTFLCKVVSLLKEVNFYSGILLLACISVDRYLA |
| | | IVHATRTLTQKRYLVKFICLSIWGLSLLLALPVLLFRRTVYSSNVSP |
| | | ACYEDMGNNTANWRMLLRILPQSFGFIVPLLIMLFCYGFTLRTLFK |
| | | AHMGQKHRAMRVIFAVVLIFLLCWLPYNLVLLADTLMRTQVIQET |
| | | CERRNHIDRALDATEILGILHSCLNPLIYAFIGQKFRHGLLKILAIHGL |
| | | ISKDSLPKDSRPSFVGSSSGHTSTTL |
| 49 | CAR-C32 | EVKFEESGGGLVQPGGSIKLSCAASGVTFSEAWMDWVRQSPEKGL |
| | | EWVAEIRDKPNSYVTYYAESVKGRFTISRDDSKSRVYLQMNNLRT |
| | | EDTGIYYCTGAMDYWGQGTSVTVSSGGGGSDIVMTQTPLSLSVTIG |
| | | QPASISCKSSQSLLYSNGKTYLNWLQQRPGQSPKRLIYQVSKLDPGI |
| | | PDRFSGSGSETDFTLKISRVEAEDLGIYYCMQGSSLPWTFGGGTKLE |
| | | IKASSHFVPVFLPAKPTTTPAPRPPTPAPTIASQPLSLRPEACRPAAG |
| | | GAVHTRGLDFACDFWVLVVVGGVLACYSLLVTVAFIIFWVRSKRS |
| | | RLLHSDYMNMTPRRPGPTRKHYQPYAPPRDFAAYRSRVKFSRSAD |
| | | APAYQQGQNQLYNELNLGRREEYDVLDKRRGRDPEMGGKPQRRK |
| | | NPQEGLYNELQKDKMAEAYSEIGMKGERRRGKGHDGLYQGLSTA |
| | | TKDTYDALHMQALPPRGSGATNFSLLKQAGDVEENPGPMCHQQL |
| | | VISWFSLVFLASPLVAIWELKKDVYVVELDWYPDAPGEMVVLTCD |
| | | TPEEDGITWTLDQSSEVLGSGKTLTIQVKEFGDAGQYTCHKGGEVL |
| | | SHSLLLLHKKEDGIWSTDILKDQKEPKNKTFLRCEAKNYSGRFTCW |
| | | WLTTISTDLTFSVKSSRGSSDPQGVTCGAATLSAERVRGDNKEYEY |
| | | SVECQEDSACPAAEESLPIEVMVDAVHKLKYENYTSSFFIRDIIKPDP |
| | | PKNLQLKPLKNSRQVEVSWEYPDTWSTPHSYFSLTFCVQVQGKSK |
| | | REKKDRVFTDKTSATVICRKNASISVRAQDRYYSSSWSEWASVPCS |
| 50 | P40 | MCHQQLVISWFSLVFLASPLVAIWELKKDVYVVELDWYPDAPGEM |
| | | VVLTCDTPEEDGITWTLDQSSEVLGSGKTLTIQVKEFGDAGQYTCH |
| | | KGGEVLSHSLLLLHKKEDGIWSTDILKDQKEPKNKTFLRCEAKNYS |
| | | GRFTCWWLTTISTDLTFSVKSSRGSSDPQGVTCGAATLSAERVRGD |
| | | NKEYEYSVECQEDSACPAAEESLPIEVMVDAVHKLKYENYTSSFFI |
| | | RDIIKPDPPKNLQLKPLKNSRQVEVSWEYPDTWSTPHSYFSLTFCVQ |
| | | VQGKSKREKKDRVFTDKTSATVICRKNASISVRAQDRYYSSSWSE |
| | | WASVPCS* |
| 51 | P2A | GSGATNFSLLKQAGDVEENPGP |
| 52 | T2A | GSGEGRGSLLTCGDVEENPGP |
| 53 | CXCR2 | MEDFNMESDSFEDFWKGEDLSNYSYSSTLPPFLLDAAPCEPESLEIN |
| | | KYFVVIIYALVFLLSLLGNSLVMLVILYSRVGRSVTDVYLLNLALA |
| | | DLLFALTLPIWAASKVNGWIFGTFLCKVVSLLKEVNFYSGILLLACI |
| | | SVDRYLAIVHATRTLTQKRYLVKFICLSIWGLSLLLALPVLLFRRTV |
| | | YSSNVSPACYEDMGNNTANWRMLLRILPQSFGFIVPLLIMLFCYGF |
| | | TLRTLFKAHMGQKHRAMRVIFAVVLIFLLCWLPYNLVLLADTLMR |
| | | TQVIQETCERRNHIDRALDATEILGILHSCLNPLIYAFIGQKFRHGLL |
| | | KILAIHGLISKDSLPKDSRPSFVGSSSGHTSTTL |
| 54 | linker6 | GGGGSGGGGS |
| 55 | linker7 | GGGGSGGGGSGGGGS |
| 56 | linker8 | GGGGSGGGGSGGGGSGGGGS |
| 57 | linker9 | GGGGS |

### Cell Lines and Cell Culture

The following cell lines were purchased from National Collection of Authenticated Cell Cultures: the human ovarian cancer cell line SK-OV-3 (Cat. No. SCSP-5214) and the human embryonic kidney cell line 293T (Cat. No. SCSP-502). The mantle cell lymphoma cell line JeKo-1 was purchased from ATCC (Cat. No. CRL-3006). The human glioblastoma cell line U87 was kindly provided by BecoolLab, Guangdong University of Technology, China. Commercially available U87 cells may also be used. The human acute lymphoblastic leukemia cell line Nalm-6 was purchased from Procell (Cat. No. CL-0701). The SK-OV-3-CD24-lucGFP cell line was obtained by transduction of SK-OV-3 with lucGFP and human CD24 viral particles, followed by culture in McCoy's 5A medium (BasalMedia, Cat. No. L630KJ), supplemented with 10% FBS (ExCell Bio, Cat. No. FSP500). The 293T cells were cultured in HEK293 Cell Complete Medium (Sinobiological, Cat. No. M293TII), which contains 10% FBS. The JeKo-1-lucGFP cell line was similarly generated by sequential transduction of JeKo-1 with lucGFP and human CD24 viral particles and subsequently cultured in in RPMI-1640 medium (Hyclone, Cat. No. SH30255.01), supplemented with 10% FBS. The Nalm-6-lucGFP cell line was obtained by transducing Nalm-6 with lucGFP viral particles and culturing it in RPMI-1640, supplemented with 10% FBS. The U87-CD24-lucGFP cell line was obtained by transducing U87 with lucGFP and human CD24 viral particles successively and culturing it in DMEM (Cytiva, Cat. No. SH30243.04), supplemented with 10% FBS.

### Generation of CD24-Specific CAR

The CD24-targeting human CAR construct includes the CD8a signal peptide and a chimera ScFv specific to CD24 from neoCD24 monoclonal antibodies. The extracellular spacer region consisted of either a CD8α or CD28 hinge, followed by the CD28 transmembrane domain, CD28 and 4-1BB costimulatory domains, and the CD3ζ signaling domain. The CAR cassette was inserted into the pHAGE lentiviral vector using NotI and BamHI restriction sites, and linked to an IRES-RFP reporter for identifying CAR⁺ cells. CAR-T cells C2, C4, C6, C8, C10, C12, C14, and C16 with variations mainly in the hinge region and co-stimulatory domain was constructed. CD24 binding was evaluated by flow cytometry using human neoCD24 (AcroImmune; AI-071-FITC), with binding strength quantified as the MFI relative to RFP expression. Functional activity was assessed by correlating binding strength with cytotoxicity and cytokine release.

To optimize CAR performance, C28 and C52 CAR-T cells were derived from the C8 ScFv by modifying the linker between VH and VL regions from 15 amino acids (45 bp) in the original to 5 (15 bp) in C28 and 10 (30 bp) in C52. Additionally, C50 CAR-T (also referred to as CAR-C31 or C31) cells were engineered from C28 to co-express CXCR2 using a P2A self-cleaving peptide.

### CAR-T Cell Production

To produce the lentiviral products, the desired CAR genes were cloned into the pHAGE lentiviral vector. The desired CAR plasmid (10.42 μg/μL) and lentiviral package system consisting of the packaging construct (psPAX2; 20.76 μg/μL) and envelope construct (pG2M.D; 13.84 μg/μL) in a ratio of 1.5:2:3, respectively, were mixed into 180 μg/μL of PEI and transfected into ~80% confluent 293T cells supplemented with DMEM containing 10% FBS. The virus supernatant was collected after 48 and 72 h of transfection and concentrated by ultracentrifugation (Beckman, Cat. No. 357003) at 25000 g for 2 h at 6 °C, and stored at −80 °C.

To generate human CD24-specific CAR-T cells, peripheral blood mononuclear cells (PBMCs; Shanghai Oricells, China, Donor: Z0160) from healthy donors were activated in 24-well plates pre-coated with anti-human CD3 (BioLegend, Cat. No. 317326), anti-human CD28 (BioLegend, Cat. No. 302934), and Retronectin (Takara, Cat No. T100B) at a final concentration of 5 μg/mL. Cells were cultured in X-VIVO 15 mediums (Lonza, Cat. No. 04-418Q) supplemented with 100 IU/mL IL-2 (Sihuan Shengwu, Beijing). After 48 h of activation, T cells (1×10⁶) were transduced with lentiviral particles carrying the CAR construct at a multiplicity of infection (MOI) of desired (2-5) at both 48 and 72 h post-activation. Plates were centrifuged at 500×g for 90 min at 32 °C. CAR expression was assessed by flow cytometry on d7, alongside non-transduced T cells as control.

### Flow Cytometry Analysis of CAR-T Cells and Target Cells

To assess CAR-T cell expression efficiency, CAR-T or NC cell suspension (1×10⁶ cells) was centrifuged at 500×g for 5 min, and the supernatant was discarded. The cell pellet was washed with 1 mL phosphate-buffered saline (PBS), centrifuged again at 500×g for 5 min, and the supernatant was removed. The cells were then incubated with human neoCD24 (AcroImmune; AI-071-FITC) and anti-human CXCR2 (CXCR2-APC; BioLegend, Cat. No. 320710) antibodies diluted 1:200 in 100 μL PBS for 30 min at 4 °C in the dark. After incubation, the cells were washed with 1 mL PBS, centrifuged at 500×g for 5 min, and resuspended in 300 μL PBS for flow cytometric analysis.

To evaluate target antigen expression on tumor cells (SK-OV-3-CD24-lucGFP, JeKo-1-lucGFP or Nalm-6-lucGFP), approximately 1×10⁶ cells were stained with the anti-human CD24 antibody (AcroImmune; 8066A-APC) at a 1:200 dilution and incubated at 4 °C for 30 min. After a final PBS wash, cells were resuspended in 300 μL PBS and analyzed by flow cytometry.

### T-Cell Cytotoxicity

The cytotoxic activity of CAR-T cells and NC cells was evaluated using a luciferase reporter gene assay, following the manufacturer's protocol (Yeasen, Cat. No. 11401ES80). Briefly, effector cells were resuspended in fresh medium and co-cultured with 4×10⁵ luciferase-expressing target cells at varying effector-to-target (E:T) ratios (2:1, 1:1, and 0.5:1) in a total volume of 1 mL per well in a 24-well plate. After 24 h of incubation at 37 °C, the plates were centrifuged at 500×g for 5 min. The supernatants were collected and stored at −20 °C for subsequent cytokine assay. Each well was then treated with 400 μL of cell lysis buffer and vortexed at 500 rpm for 10 min. The lysates were transferred to 1.5 mL microcentrifuge tubes and centrifuged at 12,000 rpm for 10 min. Following centrifugation, 20 μL of the supernatant was transferred into wells of a white 96-well plate in duplicate. Subsequently, 50 μL of luciferase substrate was added to each well, and luminescence was measured using a multimode plate reader. The killing efficiency of CAR-T cells was calculated relative to luminescence from target cells cultured without effector cells. Cytotoxicity (%)=100−(OD450 Value of Experiment)/(OD450 Value of Target cells)×100.

### Enzyme-Linked Immunosorbent Assay (ELISA)

Cytokine secretion assays for human IL-2 (Invitrogen, Cat. No. 88-7025-88), IFN-7 (Invitrogen, Cat. No. 88-7316-88), and TNF-α (Invitrogen, Cat. No. 88-7346-88) were conducted following the manufacturer's protocol. Briefly, flat-bottom 96-well plates were precoated overnight at 4 °C with 100 μL per well of capture antibody solution. Plates were then washed three times with 250 μL of wash buffer per well. Next, 200 μL per well of ELISA/ELISPOT diluent was added and incubated at room temperature (RT) for 1 h, followed by a single wash. Samples were diluted in 1×PBS, and the standard stock solution was prepared according to the manufacturer's instructions. Subsequently, 100 μL per well of standards and diluted samples were added to the plate and incubated either for 2 h at RT or overnight at 4 °C, followed by three washes. Then, 100 μL per well of detection antibody was added and incubated for 1 h at RT before washing three more times. Avidin-HRP (100 μL per well) was added, incubated for 30 min at RT, and the plate was washed six times. Finally, 100 μL per well of 1×TMB substrate solution was added, and the reaction was stopped with 50 μL per well of 2N hydrochloric acid (HCl) once the color changed to blue. The absorbance was measured at 450 nm using a multimode plate reader.

### Mouse Xenograft Models and In Vivo Function of CAR-T

Animal studies were performed using a standard protocol approved by the Institutional Animal Care and Use Committee (IACUC) of Guangzhou BeiGene Co. To establish the ovarian cancer model, immunodeficient NCG (GemPharmatech, T001475) or C-NKG mice (Cyagen, C001316) (4-6 weeks old) were intraperitoneally or subcutaneously injected with 2×10⁶ SK-OV-3-CD24-lucGFP cells. In MCL xenograft model, NCG or C-NKG mice (4-6 weeks old) were intravenous injected (i.v.) via the tail vein with 2×10⁶ JeKo-1-lucGFP cells. For acute lymphoblastic leukemia model, NCG mice (4-6 weeks old) were intravenous injected (i.v.) via the tail vein with 1×10⁶ NALM-6-lucGFP cells. Glioblastoma xenograft models were established by intracerebral injection of 3×10⁵ U87-CD24-lucGFP cells into C-NKG mice (4-5 weeks old).

Tumor growth was monitored weekly with calipers or by in vivo imaging system (IVIS). Tumor volume was calculated according to the formula: V=0.5×Length×Width×Width. For imaging, mice were intraperitoneally injected with 200 μL of D-Luciferin solution (15 mg/mL in PBS). After 5 min, mice were anesthetized with an inhalation anesthesia system, and images were acquired approximately 10 min post-injection in automatic acquisition mode.

Following tumor engraftment (2-3 weeks for ovarian cancer, 1 week for MCL/acute lymphoblastic leukemia models, 10-12 days for glioblastoma model), mice were randomized based on tumor volume (subcutaneous tumors, 100-150 mm³) or bioluminescence signal (disseminated tumors) and administered CAR-T cells (5×10⁶ to 2×10⁷ CAR-T cells intravenously injected for ovarian cancer, MCL or acute lymphoblastic leukemia models, 1×10⁶ to 1×10⁷ CAR-T cells intracerebrally or intravenously injected for glioblastoma model). Subsequently, the weight and size of the tumors in the mice were monitored weekly. Animals were euthanized when the volume of an individual tumor reached or exceeded 1200 mm³ or when the tumor diameter reached 15 mm. Mice were considered to have developed GVHD when they exhibited clinical signs including (1) weight loss of more than 20% from baseline body weight, (2) severe alopecia, and (3) significant hypothermia.

In Vivo Distribution of CAR-T Stained with DiD in the Ovarian Cancer Model

The density of CAR-T was adjusted to 1×10⁶ cells/mL with PBS, then 5M DiD (Beyotime, Cat. No. C1039) was added to the cells. Incubate at 4 °C in the dark for 30 min, and wash the cells twice with PBS. Required CAR-T cells were resuspended in normal saline. After 13 days of 2×10⁶ SK-OV-3-CD24-lucGFP inoculation on the back, NCG mice were grouped according to the size of the tumors, and then received tail vein injection of 6.5×10⁶ DiD labeled CAR-T cells. In vivo imaging was performed at 2 h, 16 h, 24 h, 4 days, and 6 days post-injection.

In Vivo Distribution of CAR-T Stained with Cy7 in the Ovarian Cancer Model

The density of CAR-T was adjusted to 1×10⁷ cells/mL with X-VIVO 15 medium, then 10 mM EDC (Meryer, Cat. No. 25952-53-8) and 5 mM NHS (Aladdin, Cat. No. H109330) was added to the cells. Cells were shaken in the incubator at 37 °C for 10 min. Then, 1 mM of Cy7 (Ruixibio, Cat. No. R-H-7336) was added and the cells were cultured at 37 °C overnight. Required CAR-T cells were washed twice with PBS and resuspended in normal saline. After 15 days of 2×10⁶ SK-OV-3-CD24-lucGFP inoculation on the back, C-NKG mice were grouped according to the size of the tumors, and then received tail vein injection of 1×10⁷ Cy7 labeled CAR-T cells. In vivo imaging was conducted at 5 min, 0.5 h, 2 h, and 4 h after injection.

### In Vivo Biodistribution of CAR-T in Ovarian Cancer Model

Sixteen days after SK-OV-3-CD24-lucGFP inoculation on the back, NCG mice were grouped by tumor volume and body weight into Vehicle, NC, and CAR-T groups (n=21 per group). Another 21 mice were not treated and served as non-tumor control. NC mice received 1.43×10⁷ total T cells via i.v., and C50 CAR-T mice received 1×10⁷ CAR⁺ T cells. At 2 h, 16 h, 24 h, 4 days, and 6 days post-injection, three mice per group were randomly selected and euthanized. Peripheral blood (~200 μL) and major organs were harvested to isolate single cells for analyzing CAR⁺ and T-cell maker biodistribution by flow cytometry.

In Vivo Pharmacokinetic and General Toxicity Study of CAR-T in Ovarian Cancer Model Fifteen days after SK-OV-3-CD24-lucGFP subcutaneously injected on the back, NCG mice were grouped by tumor volume and body weight into Vehicle (n=9), NC (n=6), and C50 CAR-T (n=9) groups. NC mice received 2.23×10⁷ total T cells via i.v., while C50 CAR-T mice received 1.0×10⁷ CAR⁺ T cells. Tumor growth was monitored twice weekly by caliper. Peripheral blood was collected on 1, 5, 8, 12, 16, 21, 28, and 39 days post-injections for pharmacokinetic study and general toxicity study (human and mouse cytokine release). For pharmacokinetic study, CAR-T⁺ and CD3⁺ cells were analyzed by flow cytometry following antibody staining.

For general toxicity assessment, the serum samples were used to evaluate inflammatory cytokines in human and mouse plasma using BioLegend's inflammation panels (human: Cat. No. 740809; mouse: Cat. No. 740446).

### Statistical Analysis

FlowJo V10 software was used to analyze T-cell infection efficiency. Experimental data were processed in GraphPad Prism 8.0. Survival differences between groups were assessed using the log-rank (Mantel-Cox) test. P value<0.05 was considered statistically significant. Cytotoxicity and cytokine detection were all subjected to repeated experiments. The representative data are presented and the data were expressed as mean±standard error of the mean. (Mean±SEM).

### Example 1

### Expressions of H3L3-CAR and PP6373-CAR

Anti-CD24 antibodies react with a broad spectrum of cancer cells and can be used to produce chimeric antigen receptors to endow T cells with the anticancer activity. In one embodiment, the antigen-binding fragments of CD24 were inserted into CAR-T vectors known in the art. These antigen-binding fragments include but are not limited to, for example, PP6373/H3L3 single-stranded Fv sequences (the heavy chain variable region can be as set forth in SEQ ID NO: 24, and the light chain variable region can be as set forth in SEQ ID NO: 26; or, the heavy chain variable region can be as set forth in SEQ ID NO: 23, and the light chain variable region can be as set forth in SEQ ID NO: 25), or other single-stranded anti-CD24 mAbs (alphaCD24SC). Then, constructs were inserted into gene vectors known in the art, including those derived from retroviruses, lentiviruses, adeno-associated viruses, or adenoviral vectors.

### (I) Preparation of T Lymphocytes

PBMCs (Day 0) from healthy donors were stimulated for 24 h with anti-human CD3, anti-human CD28, and recombinant human fibronectin (RetroNectin, RN). Activated T cells were mocked (control T) or infected with lentivirus carrying CD24-CAR (Day 1). On Days 3 and 5, the culture medium was supplemented to carry out enlarged cultivation on the cells. On Day 7, the cells were stained with CD24-FITC, and FITC and RFP signals were detected by flow cytometry to identify the expression of antigen-binding regions on the surfaces of T cells.

### (II) In Vitro Cytotoxic Test

In order to test the antitumor activity of CAR-T, the control T cells or CD24 CAR-T cells were co-cultured overnight with tumor cells that highly express target molecules CD24 and have luciferase activity. The lysis of tumor cells was measured by the luciferase detection kit (Yeasen) and calculated using the following formula:

Lysis %=100%−co-culture signal/tumor cell signal.

### (III) Cytokine Assay

In order to evaluate the safety of CAR-T, we measured the expression levels of interferon-gamma (IFN-γ) and interleukin-2 (IL-2) in the co-culture supernatant of CAR-T cells and tumor cells by the ELISA kit (Invitrogen).

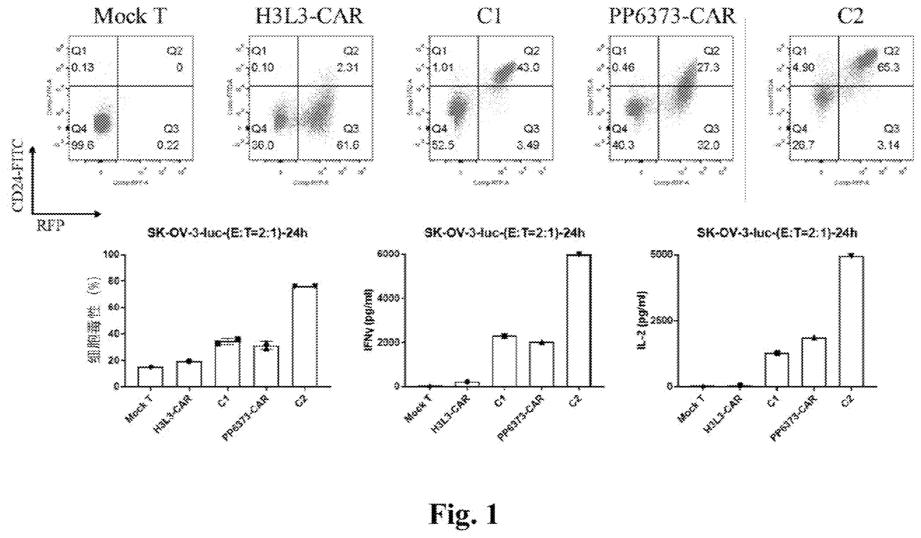

First, the structures of H3L3-CAR and PP6373-CAR were optimized by removing the first five amino acids in 4-1BB domain, to obtain C1 and C2; respectively. As shown in FIG. 1, the binding capacity of the antigen-binding regions of C1 and C2 on the surface of T cells was significantly increased. C1 and C2 also have higher antitumor activity and cytokine expression levels. The above results showed that compared with the previous H3L3-CAR and PP6373-CAR, the structurally optimized C1 and C2 could express the CAR structures more efficiently, and have stronger anti-tumor ability and higher cytokine secretion levels.

### Example 2

### Structural Optimization of CD24-CAR

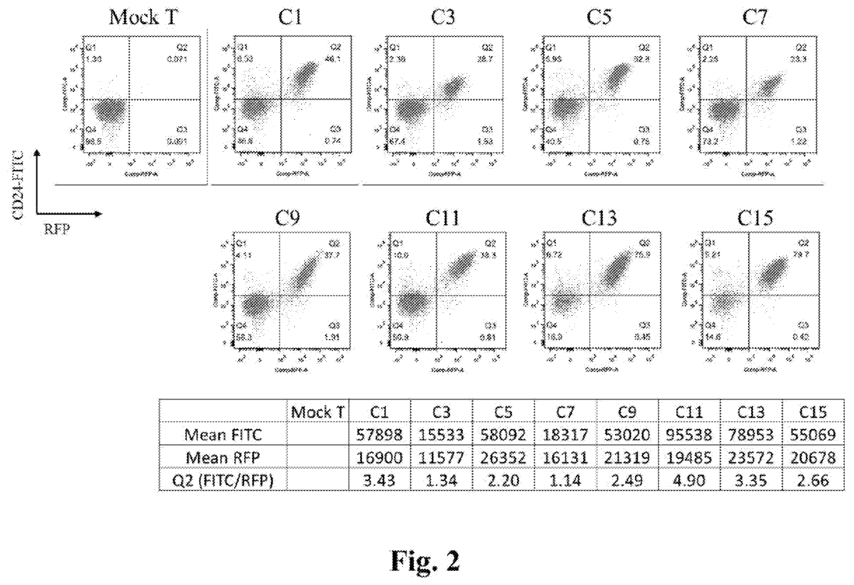

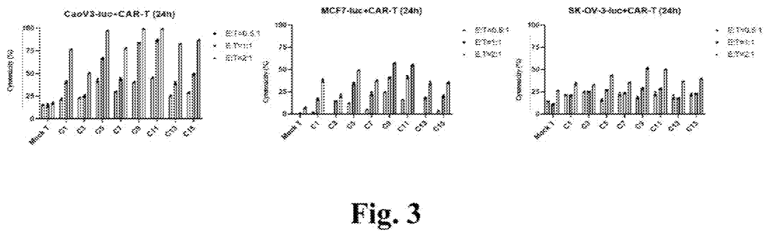

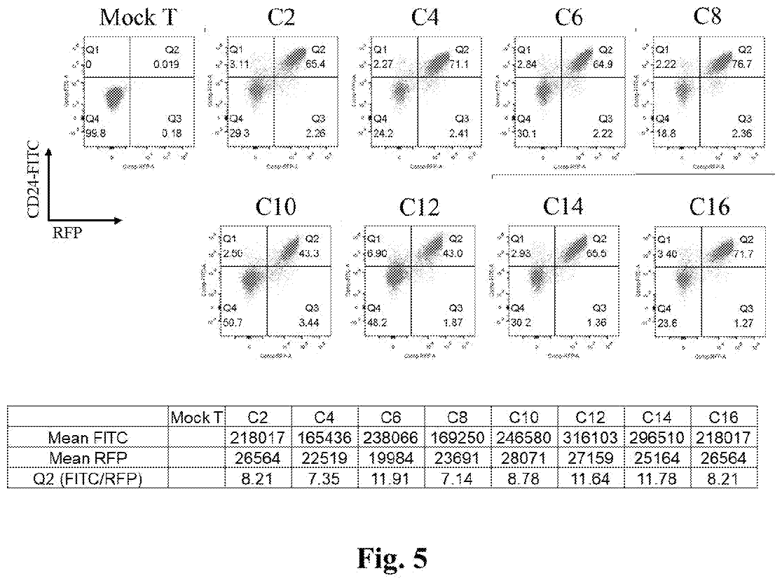

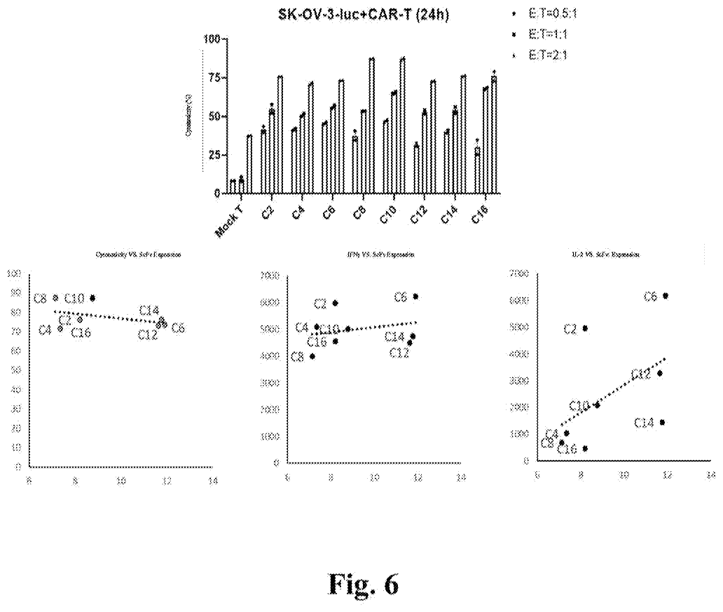

CAR structures such as C3, C5, C7, C9, C11, C13 and C15 were derived by adjusting the hinge region, transmembrane region and costimulatory domain of the CAR structure on the basis of C1 (FIG. 2). CAR structures such as C4, C6, C8, C10, C12, C14 and C16 were derived by adjusting the hinge region, transmembrane region and costimulatory domain of the CAR structure accordingly on the basis of C2 (FIG. 5). Co-culture results showed that C5, C8, C9, C10, and C11 had better antitumor activity (FIG. 3 and FIG. 6).

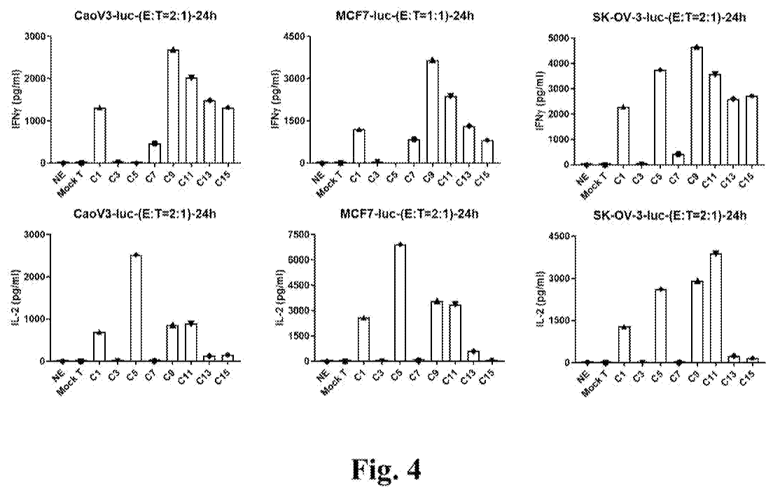

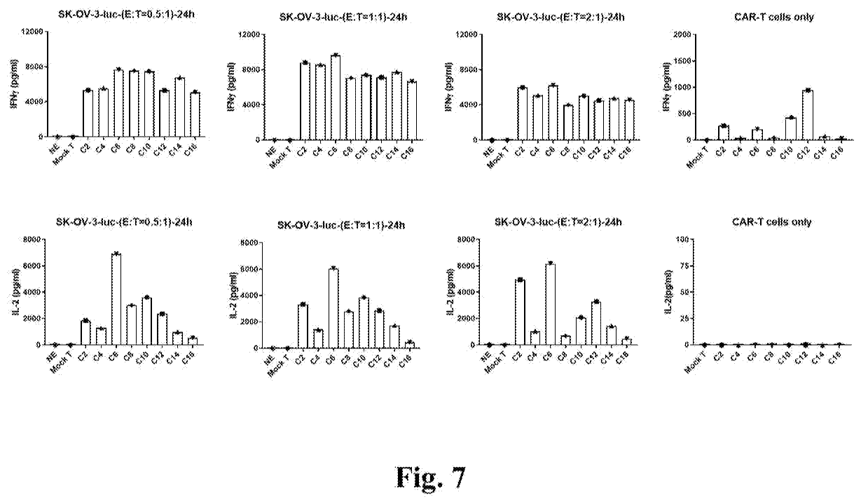

Based on further detection of cytokine secretion, it was found that C7/C8 produced IFN-7 and IL-2 at a lower level (FIG. 4, FIG. 7), indicating that the CAR structure of C7/C8 (hinge region: CD8, transmembrane region: CD28TM, and costimulatory domain: CD28) had better anti-tumor activity and better safety. It also indicated that in CD24-CAR, the second-generation CAR structure could achieve greater in vitro cytotoxic efficacy than that of the third-generation CAR.

### Example 3

### Optimization of Linker in CD24-CAR Structure

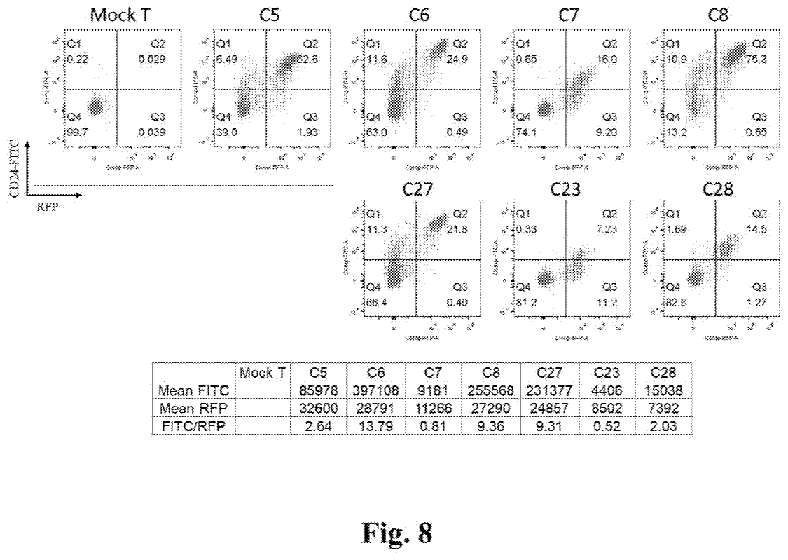

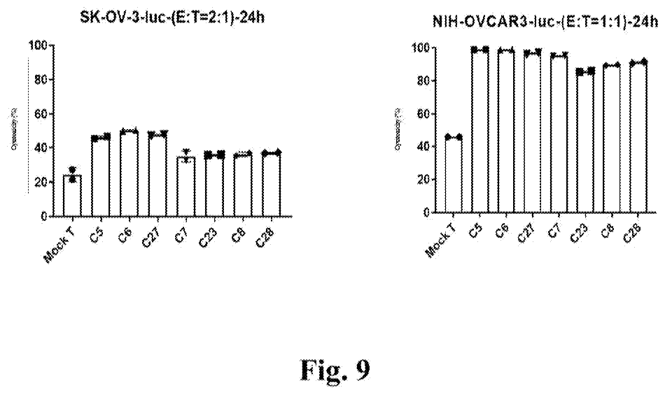

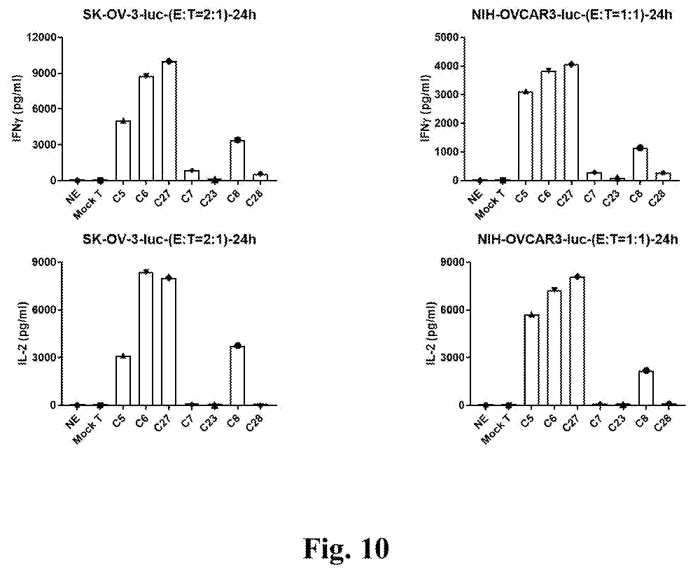

In order to further optimize the CAR structure, the linkers between the heavy and light chains of the C6 and C8 antibodies were adjusted, the amino acid sequence was shortened from (GGGGS)3 to GGGGS to obtain C27 and C28, and the C7 transmembrane region CD28TM was changed to CD8TM to obtain C23 (FIG. 8). By comparing the in vitro cytotoxic efficacy and cytokine secretion levels of these CAR structures, it was found that there was almost no difference in the in vitro cytotoxic efficacy for CAR-T cells (FIG. 9), but C28 with the shortened linker significantly reduced the cytokine secretion level (FIG. 10).

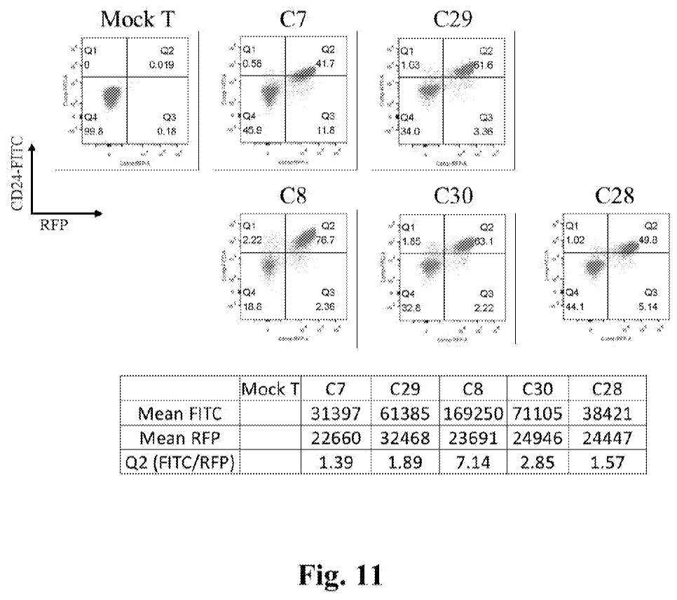

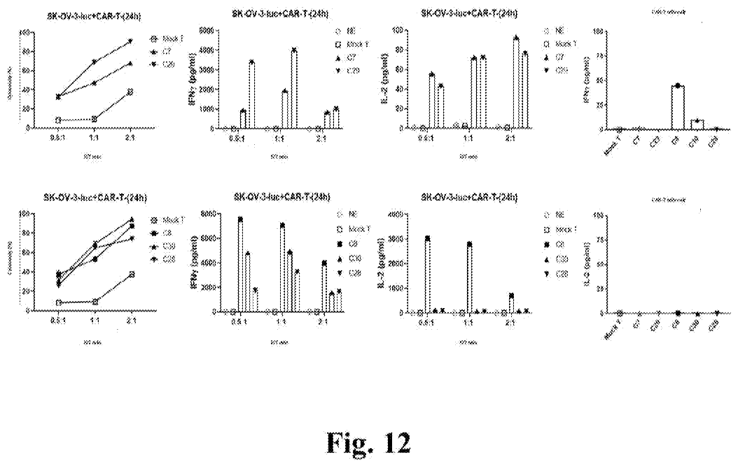

In order to further explore the effect of linker length on CAR function, the linker of C7 was extended from (GGGGS)3 to (GGGGS)4 to obtain C29; meanwhile, the linker of C8 was shortened from (GGGGS)3 to (GGGGS)2 to obtain C30 (FIG. 11). Based on in vitro experiments, we found that compared with C7, C29 with the extended linker had a significant increase in cytotoxic efficacy and IFN-7 secretion levels (FIG. 12). By comparing C8, C28 and C30, we also found that there was a positive correlation between the linker length and the IFN-7 secretion level. By comparing C8, C28 and C30, we found that there was a positive correlation between the linker length and the secretion levels of IFN-γ and IL-2, and shortening the linker length had little effect on the cytotoxic efficacy of CAR-T cells. The above results showed that we could optimize the anti-tumor effect and cytokine secretion level of CAR-T cells by modifying the linker length, and in case of high antigen-target affinity, shortening the linker could maintain the cytotoxic efficacy of CAR-T cells while significantly reduce the production of cytokines. Therefore, C28 was more effective in avoiding the risk of cytokine storm during treatment.

### Example 4

### Functional Optimization of CD24-CAR

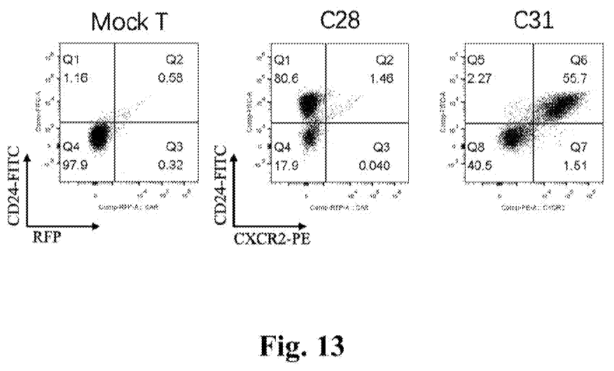

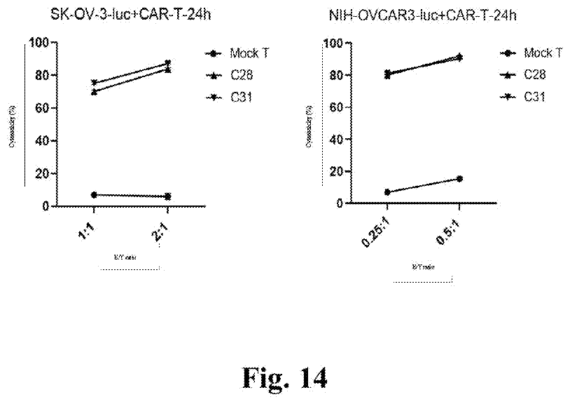

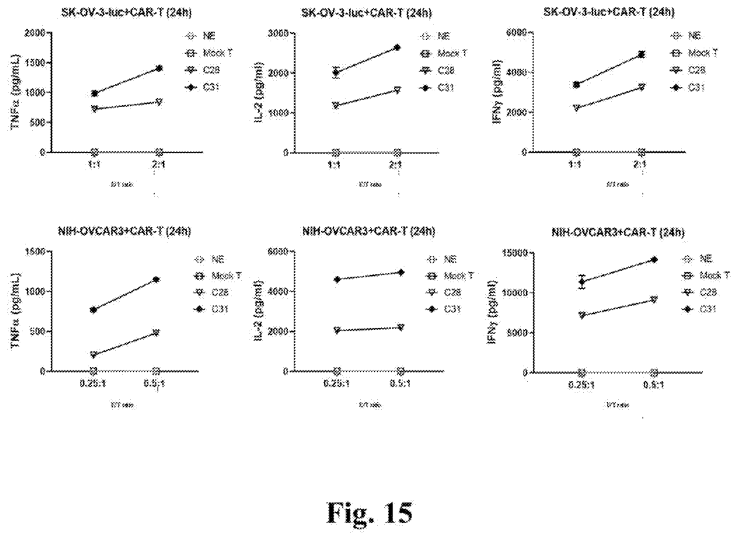

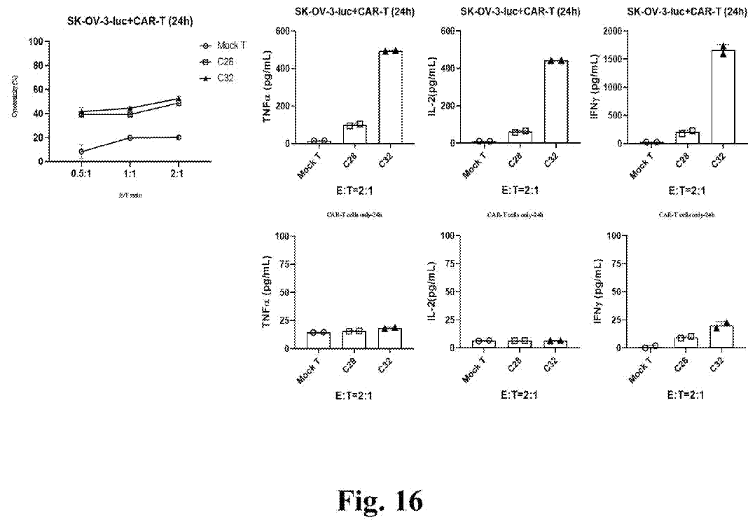

In order to enhance the chemotaxis capacity of CAR-T cells and the persistence of cytokine IFN-γ secretion, the chemokine receptor CXCR2 and cytokine IL-12β were respectively linked to the CD3ζ 3-terminal of the C28 CAR sequence via P2A linkers, to obtain C31 and C32 (FIG. 13). Based on in vitro experiments, we found that compared with C28, C31 expressing the chemokine receptor CXCR2 showed significantly increased cytotoxic efficacy for SK-OV-3 target cells, and the concentration of IFN-γ, TNF-α, IL-2 and other cytokines produced by killing target cells SK-OV-3 and NIH-OVCAR3 also increased significantly (FIG. 14 and FIG. 15). By comparing C28 and C32, we found that there was no significant difference in the in vitro cytotoxic efficacy between C32 expressing IL-12β and C28, while the secretion levels of cytokines such as IFN-γ, TNF-α, and IL-2 increased significantly (FIG. 16). The above results showed that the expression of the chemokine receptor CXCR2 helped to improve the anti-tumor effect and cytokine secretion level of C28, meanwhile IL-12β could significantly enhance the cytokine release of C28.

### Example 5

### Animal Model Test

### (I) Verification of In Vivo Functions of C8 and C28

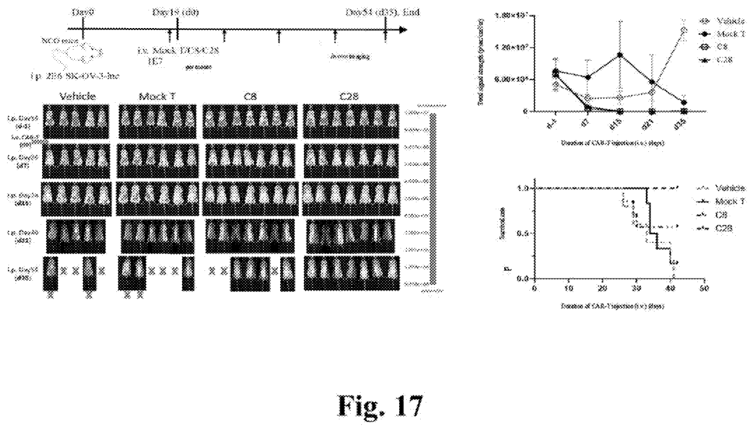

The purchased NCG immunodeficient mice of 4-5 weeks old were fed for one week in the SPF-grade animal rooms; then, each mouse was intraperitoneally injected with 2*10⁶ SK-OV-3-luc cells; 19 days after abdominal tumor formation, the intraperitoneal tumors of the mice were detected by an in vivo imager; the mice were grouped by tumor size, and injected at tail veins with 6*10⁶ C8, C28 and NC (i.e., Mock T) cells in each case and with normal saline as a blank control; and the mice were weighed weekly, and the distribution and size of tumors in these mice were monitored using the in vivo imager. As shown in FIG. 17, C8 and C28 substantially cleared tumor cells two weeks after reinfusion of CAR-T. The results showed that there was no significant difference between C8 and C28 in anti-tumor ability, and C28 had better in vivo safety.

### (II) Verification of In Vivo Migration Ability of C28 and C31

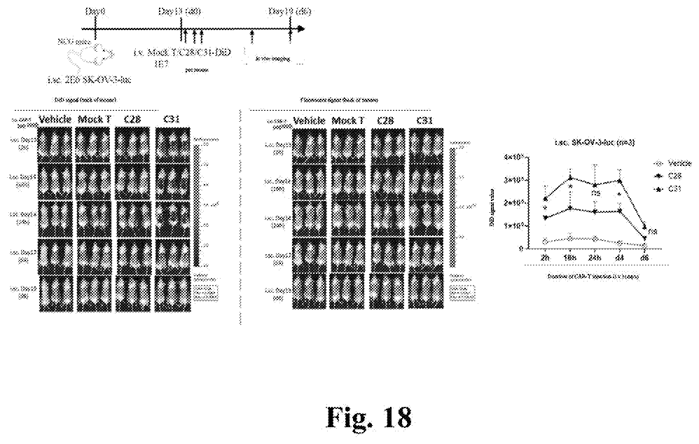

The purchased NCG immunodeficient mice of 4-5 weeks old were fed for one week in the SPF-grade animal rooms; then, each mouse was subcutaneously injected on the back with 2*10⁶ SK-OV-3-luc cells; 13 days after tumor formation, the tumors on the backs of the mice were detected by an in vivo imager; the mice were grouped by tumor size, and injected at tail veins with 1*10⁷ C28, C31 and NC (i.e., Mock T) cells tagged with the fluorescent dye DiD in each case and with normal saline as a blank control; and the CAR-T and the distribution and size of tumors in these mice were monitored using the in vivo imager. As shown in FIG. 18, the DiD signals of C31 were significantly higher than those of C28 and control cells within 6 days after CAR-T cells reinfusion, indicating that C31 had stronger in vivo migration and survival ability.

### (III) Comparison of In Vivo Functions of C28 and C31

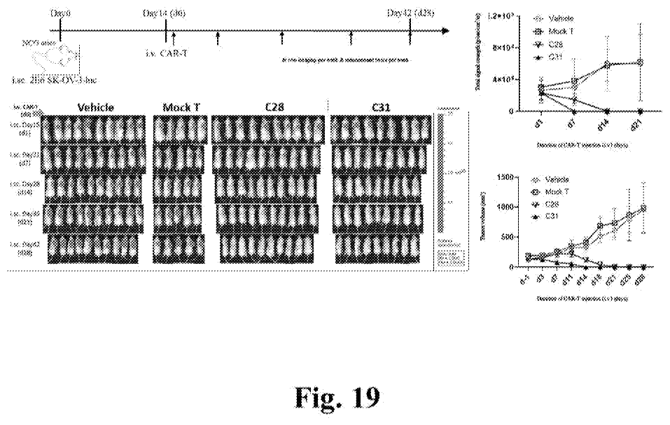

The purchased NCG immunodeficient mice of 4-5 weeks old were fed for one week in the SPF-grade animal rooms; then, each mouse was subcutaneously injected on the back with 2*10⁶ SK-OV-3-luc cells; 14 days after abdominal tumor formation, the intraperitoneal tumors of the mice were detected by an in vivo imager; the mice were grouped by tumor size, and injected at tail veins with 1*10⁷ C28, C31 and NC (i.e., Mock T) cells in each case and with normal saline as a blank control; and the mice were weighed weekly, and the distribution and size of tumors in these mice were monitored using the in vivo imager. As shown in FIG. 19, both C28 and C31 were able to clear tumor cells after reinfusion, C31 completely cleared the tumor cells in the first week after CAR-T cells reinfusion, and C28 was able to clear the tumor cells in the second week after reinfusion. There was no death of mice during the experiment, which also indicated better safety of C28 and C31.

### (IV) Comparison of In Vivo Functions of C28, C31 and C32

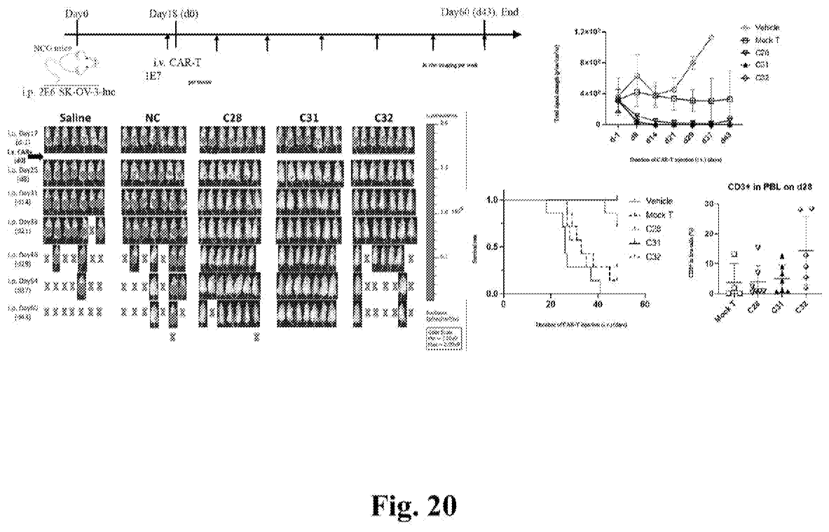

The purchased NCG immunodeficient mice of 4-5 weeks old were fed for one week in the SPF-grade animal rooms; then, each mouse was intraperitoneally injected with 2*10⁶ SK-OV-3-luc cells; 18 days after abdominal tumor formation, the intraperitoneal tumors of the mice were detected by an in vivo imager; the mice were grouped by tumor size, and injected at tail veins with 1*10⁷ C28, C31, C32 and NC (i.e., Mock T) cells in each case and with normal saline as a blank control; and the mice were weighed weekly, and the distribution and size of tumors in these mice were monitored using the in vivo imager. As shown in FIG. 20, both C31 and C32 substantially cleared tumor cells in the first week after CAR-T infusion, and completely removed the tumors in the second week after reinfusion. However, the death of mice occurred 4 weeks after T cell reinfusion with C32, and the reason for the death of mice might be the fact that C32 had good amplification capacity, and a large number of T cells appeared in the peripheral blood of NCG mice to result in GVHD to eventually cause the death of mice. Furthermore, C28 could substantially clear the tumor cells in the third week after reinfusion, indicating good tumor removal effect of C28. These results showed that C31 had better tumor removal capacity and higher in vivo safety.

In summary, the present application exemplifies the great potential of the antibody CD24 CAR-T based on the present application in cancer treatment. It has high safety and efficacy in both in vivo and in vitro experiments, without the off-target risk of killing normal PBMCs.

The foregoing detailed description is provided by way of explanation and examples, and is not intended to limit the scope of the appended claims. Various changes of the embodiments listed in the present application until now would be obvious to those of ordinary skills in the art, and should be kept within the scope of the appended claims and equivalents thereof.

### Example 6

### Antitumor Activity and Side Effects of C8 CAR-T in an Ovarian Cancer Model

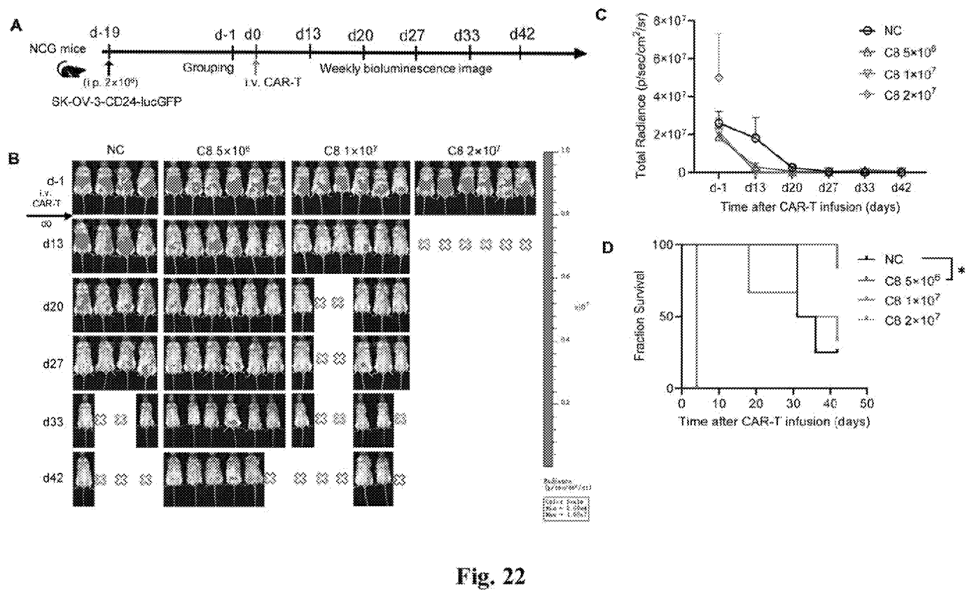

To assess antitumor efficacy in vivo, SK-OV-3-CD24-lucGFP tumor-bearing NCG mice were treated with escalating doses of C8 CAR-T cells. Bioluminescence imaging showed pronounced tumor regression in mice treated with low and medium doses of C8 CAR-T compared with the group treated with non-transduced T cells within two weeks after treatment (FIG. 22).

Mice receiving the high dose of CAR-T cells died prior to the first evaluation time point.

By Day 27, mice in the non-transduced control group also showed tumor clearance.

Survival analysis indicated that although the medium-dose group exhibited greater tumor reduction compared with the low-dose group, mice in the low-dose group showed improved overall survival relative to the other CAR-T treated groups.

These results demonstrate that C8 CAR-T exhibits antitumor activity in vivo.

### Example 7

### C50 CAR-T Cells Enhance In Vivo Antitumor Activity and Exhibit Dose-Dependent Tumor Regression in an Ovarian Cancer Xenograft Model

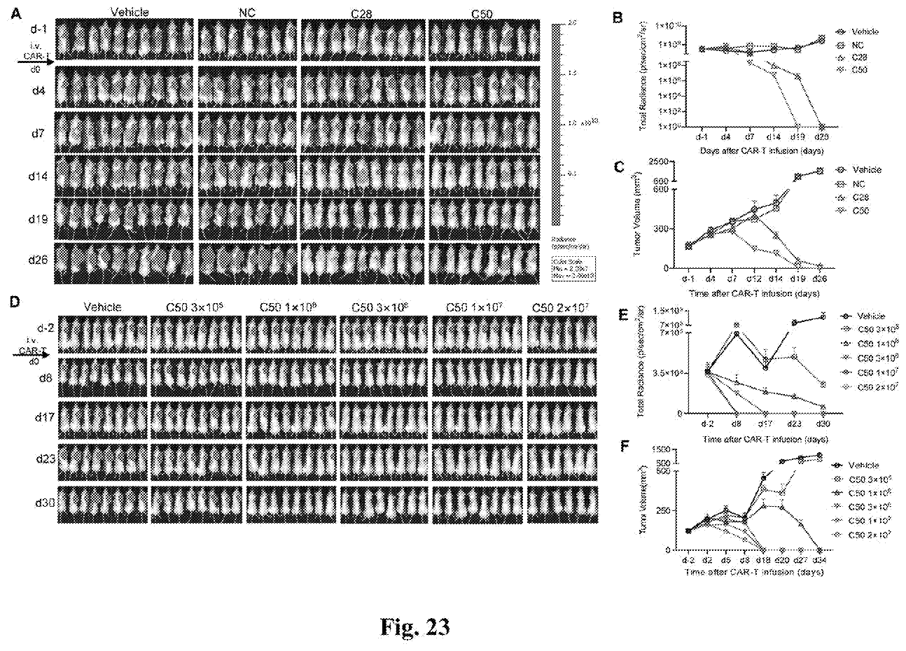

To evaluate the in vivo therapeutic potential of C50 CAR-T cells against solid ovarian tumors, SK-OV-3-CD24-lucGFP xenograft models were established via subcutaneous (s.c.) implantation. At 14 days post-treatment, mice treated with C50 CAR-T cells showed greater tumor regression than those treated with C28 CAR-T cells or control groups, as assessed by bioluminescence imaging and total radiance signal analysis (FIG. 23A-23B).

Tumor volume in the C50-treated group began to decrease at approximately day 7 following infusion and was nearly undetectable by day 19 (FIG. 23C), whereas tumor growth continued in the C28-treated group. All mice in the C50 and C28 groups survived until day 26, while rapid tumor progression was observed in the vehicle and NC groups.

To further assess dose dependence, SK-OV-3-CD24-lucGFP xenograft-bearing mice were intravenously administered C50 CAR-T cells at escalating doses (3.0×10⁵ to 2.0×10⁷ CAR⁺ cells). Bioluminescence imaging showed complete tumor elimination by day 17 in the medium- and high-dose groups (≥3.0×10⁶ CAR⁺ cells), with responses maintained throughout the observation period (FIG. 23D-23E). In contrast, mice receiving low doses (3.0×10⁵-1.0×10⁶ CAR⁺ cells) showed partial tumor regression beginning around day 8, as evidenced by residual fluorescence signals (FIG. 23F).

These observations indicate a dose-dependent antitumor effect of C50 CAR-T cells under the tested conditions.

### Example 8

### Flow Cytometric Analysis of CAR-T Cell Biodistribution and Tumor Clearance

Flow cytometric analysis was performed to characterize the biodistribution and persistence of CAR-T cells in SK-OV-3-bearing mice over a 28-day period following infusion.

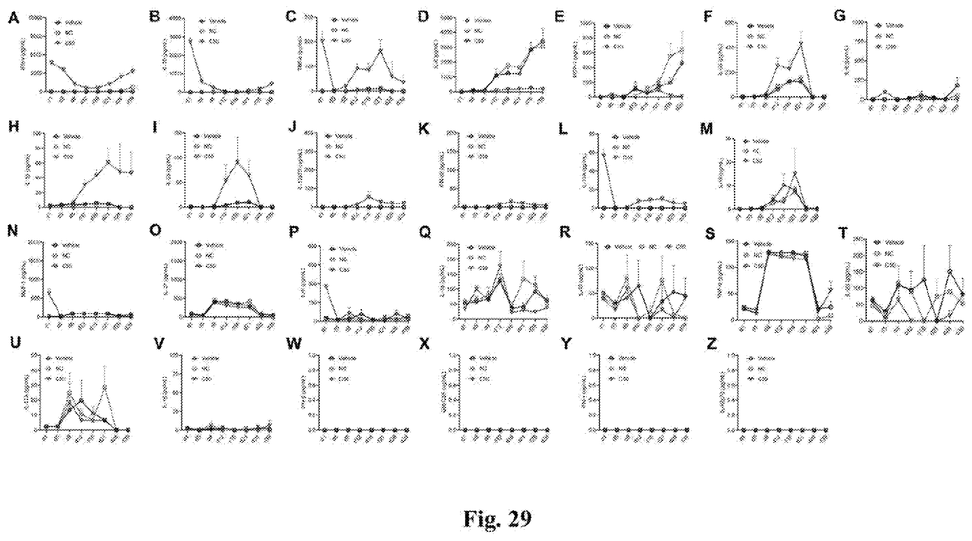

C50 CAR-T cells showed progressive expansion in peripheral blood and spleen, peaking between d14-28 (FIG. 28A-28B). Minimal CAR-T cell presence was observed in other tissues, including tumor, liver, heart, and kidney (FIG. 28C-28G), suggesting preferential proliferation within lymphoid compartments. Similar temporal patterns were observed for CD3⁺ T cells, which accumulated prominently in blood and spleen and were moderately detected in liver, lung, and kidney (FIG. 28H-28I). Within tumor tissues, CD3⁺ infiltration peaked at d14 (FIG. 28J), whereas CD45⁺ leukocytes were most abundant in liver (d14) and lung (d5-28) (FIG. 280-28P). Minimal infiltration was observed in heart and kidney (FIG. 28Q-28R). Notably, CD24⁺ tumor cells declined sharply following C50 CAR-T infusion and were completely eliminated by d14, confirming potent and selective tumor clearance (FIG. 28S).

### Example 9

### Evaluation of C50 CAR-T Cells in an Intraperitoneal Ovarian Cancer Xenograft Model

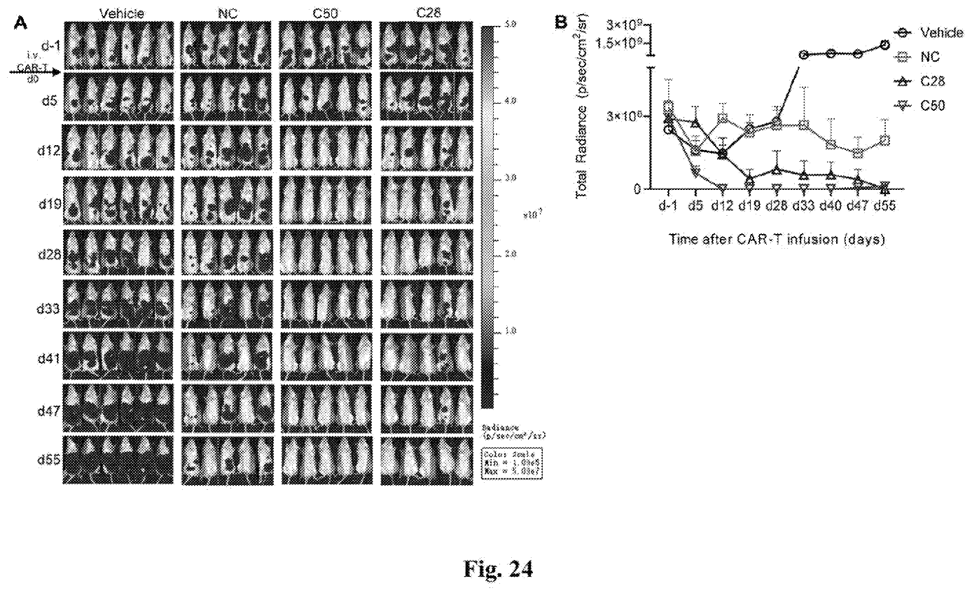

To further validate these findings across different tumor models, an intraperitoneal (i.p.) SK-OV-3-CD24-lucGFP xenograft model was established in parallel with the subcutaneous model, followed by intravenous administration of CAR-T cells. Consistent with the results observed in the subcutaneous model, C50 CAR-T treatment induced rapid and complete tumor regression within 12 days, whereas mice treated with C28 CAR-T still exhibited residual luminescence signals (FIG. 24A-24B). All C50-treated mice achieved durable remission until study completion, whereas one C28-treated mouse retained detectable tumor burden at d47. In contrast, Vehicle and NC group displayed uncontrolled tumor growth.

Overall, these results demonstrate that C50 CAR-T cells exhibit potent, dose-dependent antitumor efficacy against ovarian cancer, achieving complete tumor eradication at optimal dosing levels while maintaining favorable biodistribution and safety profiles.

### Example 10

### In Vivo Pharmacokinetics of C50 CAR-T Cells

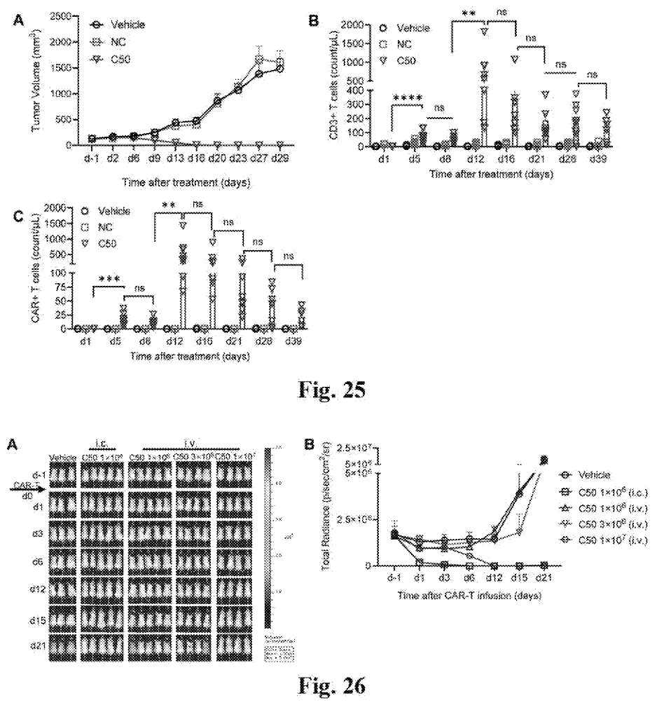

To monitor the dynamic changes of C50 CAR-T cells in peripheral blood during in vivo experiments, we performed flow cytometry analysis over a 39-day period following C50 CAR-T administration in a tumor-bearing ovarian cancer model. In the C50 CAR-T treatment group, tumor volume began to decline at d6 and was completely eradicated by d16, while tumors in the Vehicle and NC groups continued to grow uncontrollably. Notably, no tumor recurrence was observed in the C50 CAR-T group up to d29, indicating effective tumor clearance (FIG. 25A). The number of human CD3⁺ T cells in the C50 CAR-T group peaked significantly on d12, followed by a decline after d16 (FIG. 25B). In contrast, the NC group showed no detectable T-cell expansion. Consistent with CD3⁺ expression trends, the CAR⁺ in C50 CAR-T count in peripheral blood was initially low on d1 (0.3 cells/μL) after infusion, increased on d5 (16.7 cells/μL) and d8 (10.7 cells/μL), and peaked sharply on d12 (482.8 cells/μL). After d16, counts progressively decreased, reaching 21.2 cells/μL by d39 (FIG. 25C).

### Example 11

### Efficient Rejection of Glioblastoma by Either Systemic and Intracerebral Injection of C50 CAR-T

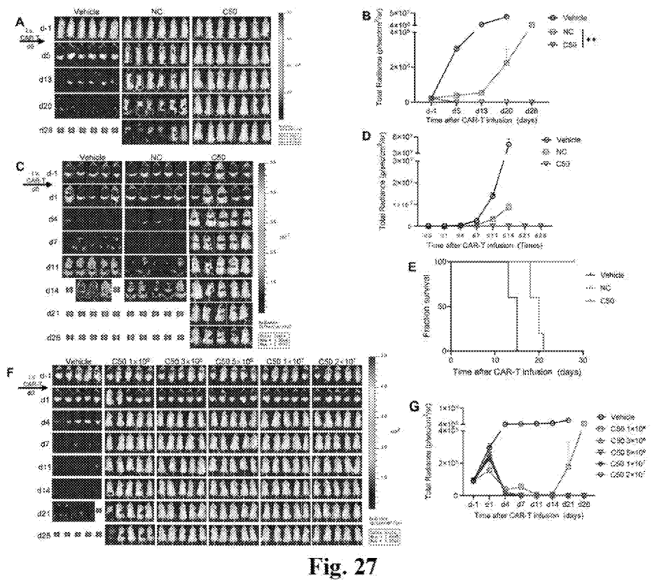

To evaluate C50 efficacy in vivo, glioblastoma xenograft models were established by intracerebral injection of 3×10⁵ U87-CD24-lucGFP cells into C-NKG mice on d-12, followed by intracerebral injection (i.e.) of 1×10⁶ or intravenous injection (i.v.) of 1×10⁶ to 1×10⁷ C50 CAR-T cells on d0. As shown in the figures (FIG. 26A-26B), C50 CAR-T treatment resulted in rapid and durable tumor regression, as evidenced by marked reduction in bioluminescence signal intensity compared with vehicle control. All mice in the Vehicle group exhibited progressive disease by d21, whereas C50-treated (i.e. 1×10⁶ and i.v. 1×10⁷) mice achieved tumor remission

### Example 12

### C50 CAR-T Cells Mediate Potent In Vivo Antitumor Activity Against Hematopoietic Malignancies

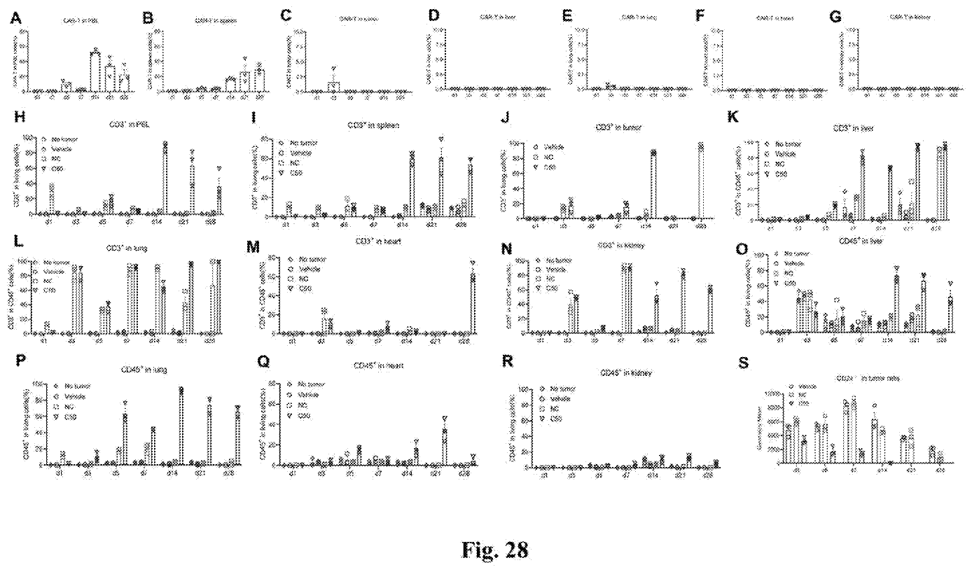

To further evaluate their efficacy in vivo, MCL xenograft models were established by intraperitoneal injection of 2×10⁶ JeKo-1-lucGFP cells into C-NKG mice on d-5, followed by infusion of 5×10⁶ C50 CAR-T cells on d0. As shown in the figures (FIG. 27A-27B), C50 CAR-T treatment resulted in rapid and durable tumor regression, as evidenced by marked reduction in bioluminescence signal intensity compared with Vehicle and non-transduced control. All mice in the Vehicle group succumbed to disease by d28, whereas C50-treated mice achieved complete tumor remission.

Consistent with these findings, C50 CAR-T cells also demonstrated robust antitumor activity in an acute lymphoblastic leukemia (ALL) xenograft model using Nalm-6-lucGFP cells (FIG. 27C-27D). Bioluminescence imaging revealed sustained tumor clearance, and all C50-treated mice survived to the study endpoint, while all control mice exhibited progressive disease and mortality (FIG. 27E).

To define the optimal therapeutic dose, JeKo-1-lucGFP MCL-bearing mice were administered C50 CAR-T cells in varying doses: 1×10⁶, 3×10⁶, 5×10⁶, 1×10⁷, and 2×10⁷ CAR-positive cells. Both the low (3×10⁶ and 5×10⁶) and high-dose (1×10⁷ and 2×10⁷) groups completely suppressed tumor growth from 4 days post-treatment until the end of the experiment (d28). In contrast, a subset of mice in the lowest-dose group (1×10⁶) exhibited tumor relapse by d14, as indicated by bioluminescence imaging (FIG. 27F-27G). These findings suggest that C50 CAR-T cells exhibit potent dose-dependent tumor control, with efficacy maintained across a broad dosing range.

### Example 13

### Safety Assessment and Cytokine Kinetics Following C50 CAR-T Cell Infusion

To evaluate safety and cytokine kinetics, plasma levels of human and murine cytokines were monitored for 42 days following CAR-T cell infusion. In the human cytokine panel, transient increases in IFN-γ (day 21-42) and IL-10 (day 0-4) were detected in the C50-treated group, whereas other cytokines remained at or near baseline levels (FIG. 29A-29M).

Similarly, in the murine cytokine panel, modest increases in IL-27, IL-23, IL-10, and TNF-α were observed, without evidence of systemic inflammatory responses (FIG. 29N-29Z).

Taken together, these findings indicate that C50 CAR-T cell treatment is associated with limited systemic cytokine elevation under the tested conditions.

1. A method for treating or alleviating a tumor, comprising administering to a subject in need thereof a therapeutically effective amount of a cell expressing a chimeric antigen receptor (CAR),
wherein the CAR comprises:
(a) a CD24-binding domain comprising a heavy chain variable region (VH) and a light chain variable region (VL),
wherein the VH comprises a HCDR1 comprising an amino acid sequence as set forth in SEQ ID NO: 42, a HCDR2 comprising an amino acid sequence as set forth in SEQ ID NO: 43, and a HCDR3 comprising an amino
acid sequence as set forth in SEQ ID NO: 44, and
wherein the VL comprises a LCDR1 comprising an amino acid sequence as set forth in SEQ ID NO: 45, a LCDR2 comprising an amino acid sequence as set forth in SEQ ID NO: 46, and a LCDR3 comprising an amino acid sequence as set forth in SEQ ID NO: 47,
wherein the VH and the VL are linked by a linker comprising an amino acid sequence selected from the group consisting of SEQ ID NOs: 27, and 54-57;
(b) a hinge region comprising a CD8 hinge region or a CD28 hinge region;
(c) a transmembrane domain comprising a CD8 transmembrane domain or a CD28 transmembrane domain; and
(d) a CD3ζ signaling domain,
wherein the tumor comprises a CD24-positive malignancy.

2. The method of claim 1, further comprising a CXCR2 co-expressed with the CAR, wherein the CXCR2 comprises an amino acid sequence as set forth in SEQ ID NO: 53.

3. The method of claim 2, wherein the CXCR2 is co-expressed with the CAR via a self-cleaving 2A peptide sequence.

4. The method of claim 1, comprising an amino acid sequence selected from the group consisting of SEQ ID NOs: 1-21 and 48-49.

5. The method of claim 1, wherein the tumor is a solid tumor or a hematopoietic malignancy.

6. The method of claim 1, wherein the tumor is selected from the group consisting of ovarian cancer, brain cancer (e.g., glioblastoma), mantle cell lymphoma (MCL), and acute lymphoblastic leukemia (ALL).

7. The method of claim 1, wherein the cell is administered intravenously, intratumorally, intrathecally, intracerebrally or systemically.

8. The method of claim 1, wherein the cell is administered as a single administration or in multiple administrations.

9. The method of claim 1, wherein the administrations are separated by an interval of about 1 day to about 12 months.

10. The method of claim 9, wherein the administrations are separated by an interval of about 1 week to about 3 months.

11. The method of claim 1, wherein the subject receives about 1×10⁵ to about 1×10¹⁰ cells.

12. The method of claim 11, wherein the subject receives about 1×10⁶ to about 1×10⁸ cells.

13. The method of claim 1, further comprising administering a lymphodepleting agent to the subject prior to administration of the cell.

14. The method of claim 1, further comprising administering an additional anti-tumor therapy.

15. The method of claim 1, wherein the cell is a T cell, NK cell, NKT cell, macrophage, or a combination thereof.

16. A chimeric antigen receptor (CAR) comprising:
(a) a CD24-binding domain comprising a heavy chain variable region (VH) and a light chain variable region (VL),
wherein the VH comprises a HCDR1 comprising an amino acid sequence as set forth in SEQ ID NO: 42, a HCDR2 comprising an amino acid sequence as set forth in SEQ ID NO: 43, and a HCDR3 comprising an amino acid sequence as set forth in SEQ ID NO: 44, and
wherein the VL comprises a LCDR1 comprising an amino acid sequence as set forth in SEQ ID NO: 45, a LCDR2 comprising an amino acid sequence as set forth in SEQ ID NO: 46, and a LCDR3 comprising an amino acid sequence as set forth in SEQ ID NO: 47,
wherein the VH and the VL are linked by a linker comprising an amino acid sequence selected from the group consisting of SEQ ID NOs: 27, and 54-57;
(b) a hinge region comprising a CD8 hinge region or a CD28 hinge region;
(c) a transmembrane domain comprising a CD8 transmembrane domain or a CD28 transmembrane domain; and
(d) a CD3ζ signaling domain.

17. The chimeric antigen receptor of claim 16, comprising an amino acid sequence selected from the group consisting of SEQ ID NOs: 1-21 and 48-49.

18. A nucleic acid molecule encoding the chimeric antigen receptor (CAR) of claim 16.

19. A cell comprising or expressing the chimeric antigen receptor (CAR) of claim 16.

20. A pharmaceutical composition comprising:
(a) a cell of claim 19; and
(b) a pharmaceutically acceptable carrier or excipient.
